(19) United States
(12) Patent Application Publication
(10) Pub. No.: US 2025/0340955 A1
(43) Pub. Date: Nov. 6, 2025

(54) COMPOSITIONS AND METHODS FOR DETECTING THERMOPHILIC ACIDOPHILIC BACTERIA IN FOODSTUFFS

(71) Applicant: THE COCA-COLA COMPANY, Atlanta, GA (US)

(72) Inventors: Marion W. SHEPHERD, JR., Atlanta, GA (US); Ewelina STEFANOVIC, Atlanta, GA (US)

(21) Appl. No.: 18/992,150

(22) PCT Filed: Jul. 7, 2023
(86) PCT No.: PCT/US2023/069835

§ 371 Date: Jan. 7, 2025

Related U.S. Application Data
(60) Provisional application No. 63/358,966, filed on Jul. 7, 2022.

(51) Int. Cl. C12Q 1/689

(52) U.S. Cl. CPC C12Q 1/689; C12Q 2600/156; C12Q 2600/16

(57) ABSTRACT

The present disclosure provides compositions, methods, and kits for detecting guaiacol-producing microorganisms, in particular, Thermophilic Acidophilic Bacteria (TAB) in foodstuff. In one example, a method comprises: (a) contacting a set of oligonucleotides with the sample, wherein the set of oligonucleotides comprising: a first collection of primers targeted for nucleic acids of a guaiacol-producing microorganism; and a second collection of primers targeted for nucleic acids of Alicyclobacillus acidoterrestris; (b) amplifying DNA in the sample with the said set of oligonucleotides under a multiplex polymerase chain reaction (PCR); and (c) determining the presence of PCR products of step (b), wherein the presence of a PCR product in the sample is indicative of contamination of the sample by both guaiacol-producing microorganism and Alicyclobacillus acidoterrestris.

This application is being filed on Jul. 7, 2023, as a PCT International Patent application and claims the benefit of and priority to U.S. Provisional patent application Ser. No. 63/358,966, filed on Jul. 7, 2022, the entire disclosure of which is incorporated by reference in its entirety.

### SUBMISSION OF SEQUENCE LISTING

The Sequence Listing associated with this application is filed in electronic format via EFS-Web and is hereby incorporated by reference into the specification in its entirety.

### INTRODUCTION

Microorganisms (e.g., bacteria) may cause the spoilage of foodstuffs (e.g., beverages) during or after manufacture. Some microorganisms may cause one or more of several undesirable effects such as unpleasant odor and unpleasant taste, rendering the foodstuff unsafe for consumption or inedible due to the presence of off-flavors. Failure to accurately and rapidly detect the presence of foodstuff-spoiling microorganisms may increase the risk of food spoilage. Challenges to the rapid and accurate detection of the microorganisms that cause the spoilage of foodstuffs may include, for example, the lengthy duration of the traditional microbiology methods used to detect the microorganisms. These traditional methods may take an average of at least five (5) days to complete.

In particular, strains Thermophilic Acidophilic Bacteria (TAB), members of the genus Alicyclobacillus, are heat-resistant, acid-tolerant, and able to survive pasteurization during the processing of beverages and ingredient use to make beverages, causing spoilage accompanied with the production of guaiacol—a compound characterized by its medical, phenolic, or antiseptic off-flavor. A. acidoterrestris is the species most associated with guaiacol production, although other species (e.g., A. acidiphilus, A. herbarius) are also able to produce this molecule. Therefore, spoilage by Alicyclobacillus is a major concern for the beverage industry. Although TAB are not pathogenic to humans, the contamination during processing and production can result in significant economic losses to the beverage industry.

Accordingly, there is a need for improved compositions and methods for detecting foodstuff-spoiling microorganisms, particularly TAB.

### SUMMARY OF DISCLOSURE

Aspects of the present disclosure may be embodied in various exemplary and nonlimiting forms. In particular, this summary is intended merely to illuminate various embodiments of the disclosure and does not impose a limitation on the scope of the disclosure.

The present disclosure provides a solution to efficient and accurate detection of TAB in a sample of foodstuff, through use of Polymerase Chain Reaction (PCR) techniques. The provided solution may be used to determine: if an organism is an Alicyclobacillus isolate (Genus-level detection); if the organism is A. acidoterrestris (Species-level determination); and/or if the organism has the gene responsible for guaiacol production.

In some aspects, the present disclosure provides methods for detecting a spoilage microorganism in a sample of foodstuff. In one example, a method comprises: (a) contacting a set of oligonucleotides with the sample, wherein the set of oligonucleotides comprising: a first collection of primers targeted for nucleic acids of a guaiacol-producing microorganism; and a second collection of primers targeted for nucleic acids of Alicyclobacillus acidoterrestris; (b) amplifying DNA in the sample with the said set of oligonucleotides under a multiplex polymerase chain reaction (PCR); and (c) determining the presence of PCR products of step (b), wherein the presence of a PCR product in the sample is indicative of contamination of the sample by both guaiacol-producing microorganism and Alicyclobacillus acidoterrestris.

In another example, a method for detecting a spoilage microorganism in a sample of foodstuff, the method comprising: (a) contacting a set of oligonucleotides with the sample, wherein the set of oligonucleotides comprising: a first collection of primers targeted for nucleic acids of a guaiacol-producing microorganism; a second collection of primers targeted for nucleic acids of Alicyclobacillus acidoterrestris; and a third primer targeted for nucleic acids of the 16 S rRNA gene common to the genus Alicyclobacillus; (b) amplifying DNA in the sample with the said set of oligonucleotides under a multiplex polymerase chain reaction (PCR); and (c) determining the presence of PCR products of step (b), wherein the presence of a PCR product in the sample is indicative of contamination of the sample by a guaiacol-producing microorganism, the specific species Alicyclobacillus acidoterrestris, and the genus Alicyclobacillus.

In some aspects, the present disclosure provides systems and assay kits for a spoilage microorganism in a sample of foodstuff. In one example, a system comprises a set of oligonucleotides for multiplex PCR, wherein the set of oligonucleotides comprises: a first collection of primers targeted for nucleic acids of a guaiacol-producing microorganism; and a second collection of primers targeted for nucleic acids of Alicyclobacillus acidoterrestris.

In another example, a system comprises a set of oligonucleotides for multiplex PCR, wherein the set of oligonucleotides comprises: a first collection of primers targeted for nucleic acids of a guaiacol-producing microorganism; a second collection of primers targeted for nucleic acids of Alicyclobacillus acidoterrestris, and a third collection of primers targeted for nucleic acids of the 16 S rRNA gone common to the genus Alicyclobacillus.

In yet another example, a kit for detecting a spoilage microorganism in a sample of foodstuff, the kit comprising: a first collection of primers targeted for nucleic acids of a guaiacol-producing microorganism; and a second collection of primers targeted for nucleic acids of Alicyclobacillus acidoterrestris. In some embodiments, the kit further comprises: an instruction providing a method, the method comprising: (a) contacting the set of oligonucleotides with the sample; (b) amplifying DNA in the sample with the said set of oligonucleotides under a multiplex polymerase chain reaction (PCR); and (c) determining the presence of PCR products of step (b), wherein the presence of a PCR product in the sample is indicative of contamination of the sample by a guaiacol-producing microorganism and the specific species Alicyclobacillus acidoterrestris.

In a further example, a kit for detecting a spoilage microorganism in a sample of foodstuff, the kit comprising: a first collection of primers targeted for nucleic acids of a guaiacol-producing microorganism; a second collection of primers targeted for nucleic acids of Alicyclobacillus acidoterrestris, and a third collection of primers targeted for nucleic acids of the 16 S rRNA gene common to the genus Alicyclobacillus. In some embodiments, the kit further comprises: an instruction providing a method, the method comprising: (a) contacting the set of oligonucleotides with the sample; (b) amplifying DNA in the sample with the said set of oligonucleotides under a multiplex polymerase chain reaction (PCR); and (c) determining the presence of PCR products of step (b), wherein the presence of a PCR product in the sample is indicative of contamination of the sample by a guaiacol-producing microorganism, the specific species Alicyclobacillus acidoterrestris, and the genus Alicyclobacillus.

In some embodiments, the guaiacol-producing gene encodes vanillin decarboxylase (vdc). In some embodiments, the guaiacol-producing gene encoding vanillin decarboxylase (vdc) has at least 75% identity to the nucleic acid sequence set forth in SEQ ID NO. 51.

In some embodiments, the second collection of primers is targeted for the rpoB gene and/or the gyrB gene of Alicyclobacillus acidoterrestris. In some embodiments, the rpoB gene comprises a nucleic acid sequence that has at least 75% identity to the nucleic acid sequence set forth in SEQ ID NOS 9-14. In some embodiments, the gyrB gene comprises a nucleic acid sequence that has at least 75% identity to the nucleic acid sequence set forth in SEQ ID NOS 15-26.

In some embodiments, the 16 S rRNA gene comprises a nucleic acid sequence that has at least 75% identity to the nucleic acid sequence set forth in SEQ ID NOS. 27-50.

In some embodiments, the first collection of primers comprises at least one primer having at least 75% identity to the nucleic acid sequence set forth in SEQ ID NOS 1-2.

In some embodiments, the second collection of primers comprises at least one primer having at least 75% identity to the nucleic acid sequence set forth in SEQ ID NOS. 3.6.

In some embodiments, the third collection of primers comprises at least one primer having at least 75% identity to the nucleic acid sequence set forth in SEQ ID NOS. 7-8.

In some embodiments, the first collection of primers hybridizes with the nucleic acids of the guaiacol-producing gene. In some embodiments, the second collection of primers hybridizes with the nucleic acids of the Alicyclobacillus acidoterrestris. In some embodiments, the third collection of primers hybridizes with the nucleic acids of the 16 S rRNA gene.

In some embodiments, the second collection of primers comprises at least one of: a forward primer having at least 75% identity to the nucleic acid sequence set forth in SEQ ID NOS. 3 and 5.

In some embodiments, the second collection of primers comprises at least one of: a reverse primer having at least 75% identity to the nucleic acid sequence set forth in SEQ ID NOS. 4 and 6.

In some embodiments, the third collection of primers comprises at least one of: a forward primer having at least 75% identity to the nucleic acid sequence set forth in SEQ TD NO. 7. In some embodiments, the third collection of primers comprises at least one of a reverse primer having at least 75% identity to the nucleic acid sequence set forth in SEQ ID NO. 8.

In some embodiments, each primer of the first collection of primers has a length of no more than 35 nucleotides. In some embodiments, each primer of the second collection of primers has a length of no more than 35 nucleotides. In some embodiments, each primer of the third collection of primers has a length of no more than 35 nucleotides.

In some embodiments, the spoilage microorganism detected by the method comprise at least one of Alicyclobacillus acidocaldarius, Alicyclobacillus contaminans, Alicyclobacillus disulfidooxidans, Alicyclobacillus fastidiosus, Alicyclobacillus ferrooxydans, Alicyclobacillus hesperidum, Alicyclobacillus pomorum, Alicyclobacillus sacchari, Alicyclobacillus sendaiensis, Alicyclobacillus shizuokensis, Alicyclobacillus tolerans, Alicyclobacillus vulcanalis, Alicyclobacillus tengchongenesis, Alicyclobacillus acidoterristris, Alicyclobacillus acidiphilus, Alicyclobacillus cycloheptanicus, and Alicyclobacillus herbarius.

In some embodiments, the foodstuff is a beverage, a drink, a juice, or a fruit juice.

In some embodiments, the PCR is quantitative PCR or a real-time PCR.

In some embodiments, the multiplex PCR reaction takes about 5 days or less, about 4 days or less, about 3 days or less, about 2 days or less, or about 1 day or less.

### BRIEF DESCRIPTION OF THE DRAWINGS

FIG. 1 illustrates an example of gene sequence alignment of SEQ ID NOS. 9-14.

FIG. 2 illustrates an example of gene sequence alignment of SEQ ID NOS. 15-26.

FIG. 3 illustrates an example of gene sequence alignment of SEQ ID NOS. 27-50.

FIG. 4A illustrates the structure of the vdc region and positions of genetic elements and selected tested primers according to the present disclosure.

FIG. 4B illustrates the gel electrophoresis of PCR amplicons obtained by Bur5/6 primers (SEQ ID NOS, 1-2). Stains labelled in red are guaiacol producers, strains labelled in white do not produce guaiacol (Doehler test kit).

FIG. 5 illustrates gel electrophoresis results of the multiplex 2 PCR reactions for guaiacol production and specific for A. acidoterrestris, using a combination of primers having sequences set forth in SEQ ID NOS. 1-6.

FIG. 6 illustrates gel electrophoresis results of multiplex 3 PCR reactions, using a combination of primers having sequences set forth in SEQ ID NOS. 1-8. A. acidoterrestris(AT) showed 3 bands—species specific (gyrB); guaiacol producing (Bur5/6); and genus specific (AGES). Non-AT strains showed single band (AGES).

### BRIEF DESCRIPTION OF THE SEQUENCE LISTINGS

SEQ ID NO: 1 discloses the sequence of primer Bur5 (5′-3′) targeted for the Vanillin decarboxylase (vdc) region of A. acidoterrestris:

CCGACGTGATGCTCAARGAGCGCA.

SEQ ID NO: 2 discloses the sequence of primer Bur6 (5′-3′) targeted for the Vanillin decarboxylase (vfc) region of A. acidoterrestris:

GTSGCRTCGAGAATCATCTTGTG.

SEQ ID NO: 3 discloses the sequence of forward primer for rpoB gene (5′-3′):

CCGTCGTGAGGACGTTATCGCGGTGCCG.

SEQ ID NO: 4 discloses the sequence of reverse primer for rpoB gene (5′-3′):

CGCAACGTCACCGCGAACAAGTCGG.

SEQ ID NO: 5 discloses the sequence of forward primer for gyrB gene (5′-3′):

GTTCCCAAGTACGATTTGAAAGTGG.

SEQ ID NO: 6 discloses the sequence of reverse primer for gyrB gene (5′-3′):

GGTTGCTCTCGCTCCCCTTAATC.

SEQ ID NO: 7 discloses the sequence of forward primer for AGES (5′-3′):

GTYRCTCGGGRAGAGCG.

SEQ ID NO: 8 discloses the sequence of reverse primer for AGES (5′-3′):

CGCCGRCAGTCACCTGT.

SEQ ID NO: 9 discloses the sequence of rpoB gene of Alicyclobacillus acidocaldarius.

SEQ ID NO: 10 discloses the sequence of rpoB gene of Alicyclobacillus mali.

SEQ ID NO: 11 discloses the sequence of rpoB gene of Alicyclobacillus acidoterrestris ATCC_49025.

SEQ ID NO: 12 discloses the sequence of rpoB gene of Alicyclobacillus acidiphilus.

SEQ ID NO: 13 discloses the sequence of rpoB gene of Alicyclobacillus sacchari.

SEQ ID NO: 14 discloses the sequence of rpoB of gene of Alicyclobacillus hesperidum.

SEQ ID NO: 15 discloses the sequence of gyrB gene of Alicyclobacillus contaminans ACON_DSMI7975.

SEQ ID NO: 16 discloses the sequence of gyrB gene of Alicyclobacillus acidoterrestris AT_NBRC 106287.

SEQ ID NO: 17 discloses the sequence of gyrB gene of Alicyclobacillus acidoterrestris AT_ATCC49025.

SEQ ID NO: 18 discloses the sequence of gyrB gene of Alicyclobacillus acidoterrestris AT_ATCC49025_2.

SEQ ID NO: 19 discloses the sequence of gyrB gene of Alicyclobacillus acidoterrestris AT_OR3.

SEQ ID NO: 20 discloses the sequence of gyrB gene of Alicyclobacillus hesperidum AH_DSM 12489.

SEQ ID NO: 21 discloses the sequence of gyrB gene of Alicyclobacillus sacchari ASach_DSM974.

SEQ ID NO: 22 discloses the sequence of gyrB gene of Alicyclobacillus acidiphilus AP_NBRC 100859.

SEQ ID NO: 23 discloses the sequence of gyrB gene of Alicyclobacillus acidiphilus AP_nondefined.

SEQ ID NO: 24 discloses the sequence of gyrB gene of Alicyclobacillus acidocaldarius AC_DSM446.

SEQ ID NO: 25 discloses the sequence of gyrB gene of Alicyclobacillus sendaiensis AS NBRC 100866.

SEQ ID NO: 26 discloses the sequence of gyrB gene of Alicyclobacillus sendaiensis AS_nondefined.

SEQ ID NO: 27 discloses the sequence of 16 S rRNA of B. subtilis 168.

SEQ ID NO: 28 discloses the sequence of 16 S rRNA of G. stearothermophilus R-32640.

SEQ ID NO: 29 discloses the sequence of 16 S rRNA of D. stibiiarsenatis MLFW-2.

SEQ ID NO: 30 discloses the sequence of 16 S rRNA of A. contaminans 3 A191.

SEQ ID NO: 31 discloses the sequence of 1.6 S rRNA of A. contaminanas NRRC103102.

SEQ ID NO: 32 discloses the sequence of 16 S rRNA of A. sendaiensis JCM1181.

SEQ ID NO: 33 discloses the sequence of 16 S rRNA of A. sendaiensis NBRC100866.

SEQ ID NO: 34 discloses the sequence of 16 S rRNA of A. sendaiensis NTAP-1.

SEQ ID NO: 35 discloses the sequence of 16 S rRNA of A. acidocaldarius rittmanni DSM11297.

SEQ ID NO: 36 discloses the sequence of 16 S rRNA of A. acidocaldarius rittmanni MR1.

SEQ ID NO: 37 discloses the sequence of 16 S rRNA of A. acidocaldarius acidocaldarius DSM446.

SEQ ID NO: 38 discloses the sequence of 16 S rRNA of A. acidocaldarius 104-1A.

SEQ ID NO: 39 discloses the sequence of 16 S rRNA of A. acidocaldarius acidocaldarius ATCC27009.

SEQ ID NO: 40 discloses the sequence of 16 S rRNA of A. acidocaldarius NBRC15652.

SEQ ID NO: 41 discloses the sequence of 16 S rRNA of A. acidiphilus NBRC100859.

SEQ ID NO: 42 discloses the sequence of 16 S rRNA of A. acidiphilus TA-67.

SEQ ID NO: 43 discloses the sequence of 16 S rRNA of A. sacchari NBRC 103105.

SEQ ID NO: 44 discloses the sequence of 16 S rRNA of A. sacchari R8718.

SEQ ID NO: 45 discloses the sequence of 16 S rRNA of A. hesperidium DW12489.

SEQ ID NO: 46 discloses the sequence of 16 S rRNA of A. hesperidum FR-11

SEQ ID NO: 47 discloses the sequence of 16 S rRNA of A. acidoterrestris DSM3922.

SEQ ID NO: 48 discloses the sequence of 16 S rRNA of A. acidoterrestris ATCC49025.

SEQ ID NO: 49 discloses the sequence of 1.6 S rRNA of A. fastidiosus NBR10309.

SEQ ID NO: 50 discloses the sequence of 16 S rRNA of A. fastidiosus S-TAB.

SEQ ID NO: 51 discloses the sequence of the vdc region of guaiacol-producing microorganisms.

### DETAILED DESCRIPTION

According to the methods described herein, a sample is obtained from a test material, for example a sample of a beverage, a fruit juice, or other food product. The sample is processed to extract any polynucleotides in the sample, particularly polynucleotides from target microorganisms that may be present in the material. After extraction and processing according to methods described herein and/or otherwise known in the art, the sample is treated with a composition that comprises a component having at least one of: a forward primer oligonucleotide, a reverse primer oligonucleotide, and/or a labeled oligonucleotide probe, wherein the composition is targeted for specific regions within the genome of target organisms. The sample is then processed according to polymerase chain reaction (PCR), particularly multiplex PCR amplification methods. The PCR product is first amplified using the primers. Binding of the labeled probe to a target sequence within the PCR product that corresponds with a target region in the genomic DNA of the contaminating bacteria signals the presence of contaminating microorganisms. Therefore the combination of the present composition and the PCR technology ensured specific, rapid, and sensitive detection of the presence of the target bacteria. The solutions provided in the present disclosure allows for efficient and accurate determination of: if an organism is an Alicyclobacillus isolate (Genus-level detection), if the organism is A. acidoterrestris (Species-level determination), and if the isolate has the gene responsible for guaiacol production, through use of multiplex PCR.

### Primer Selection

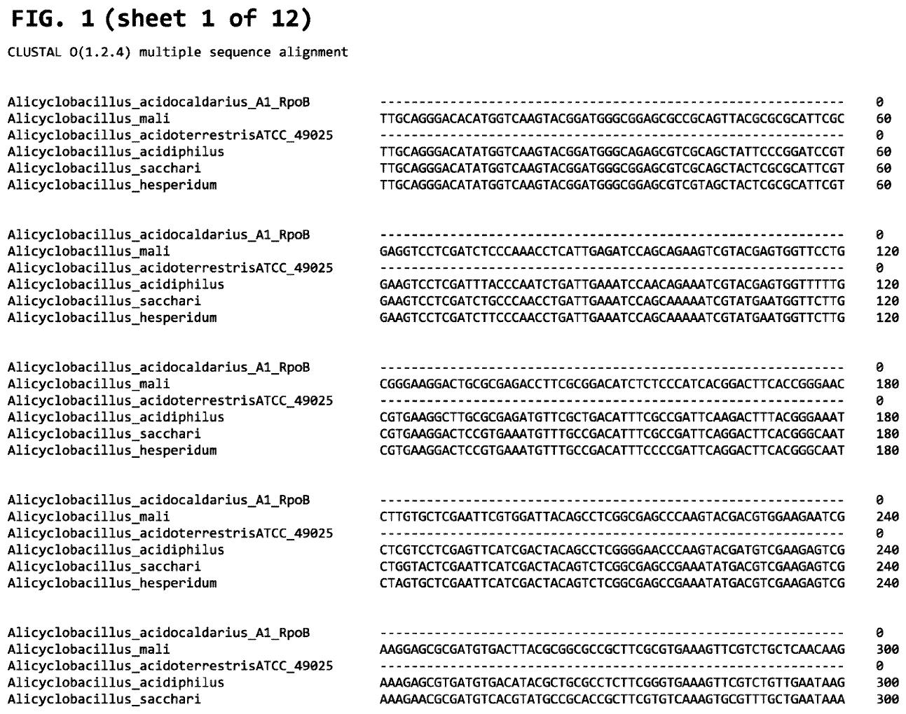

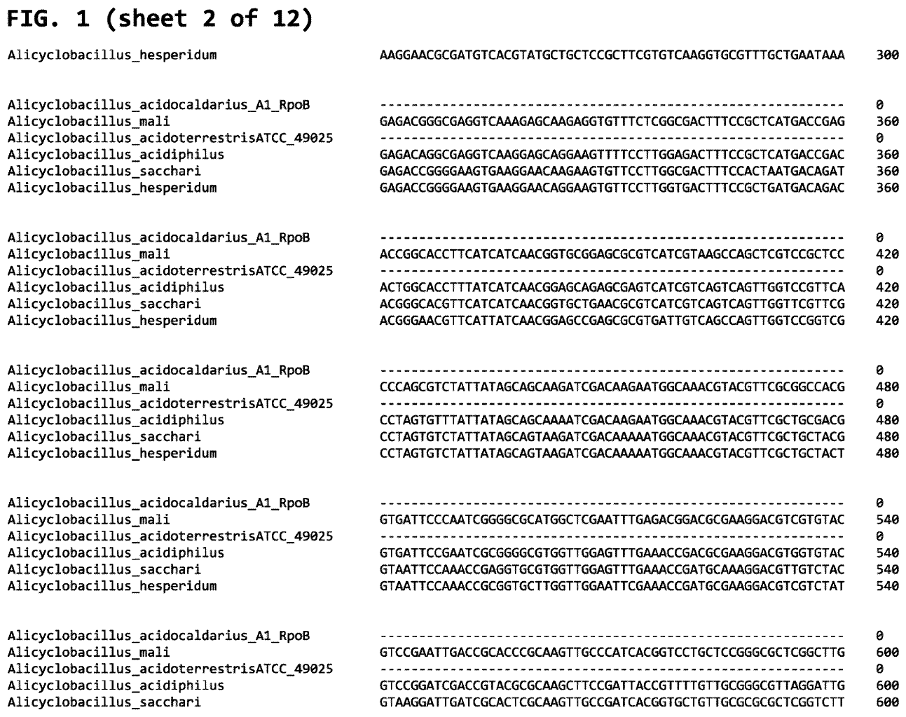

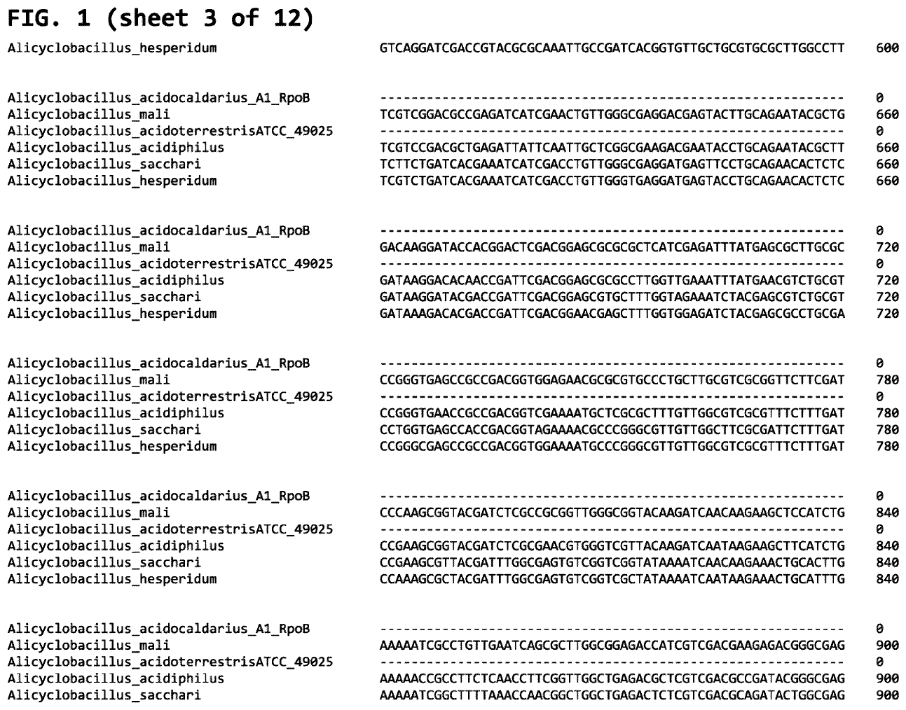

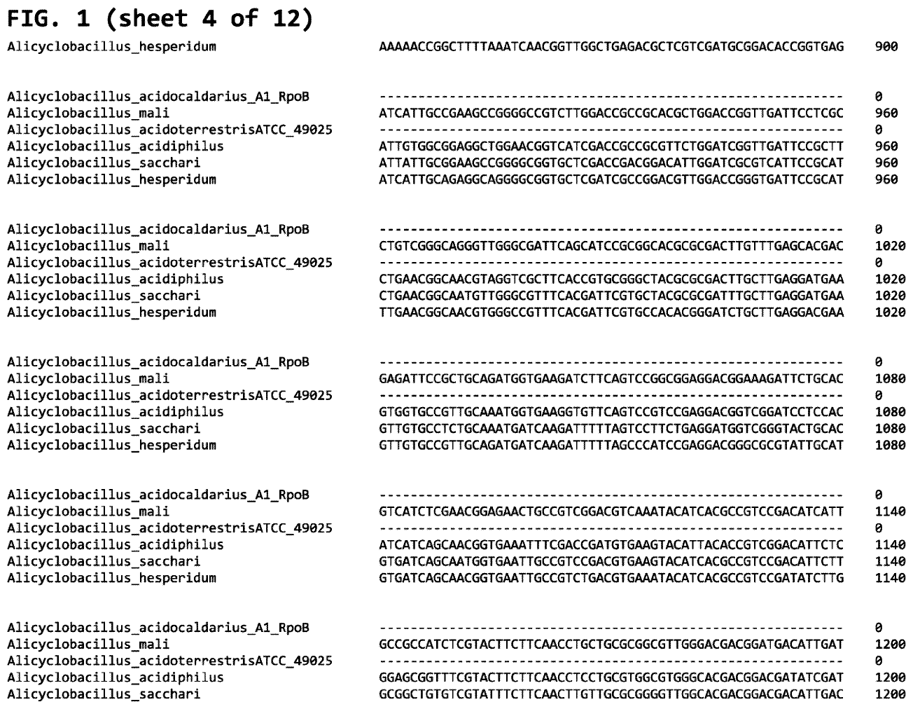

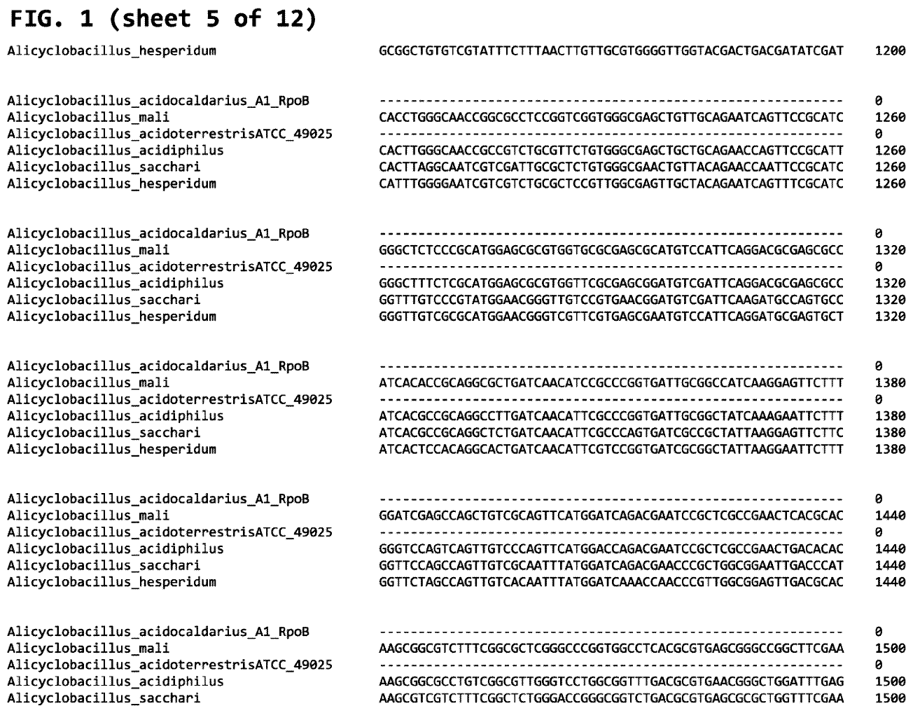

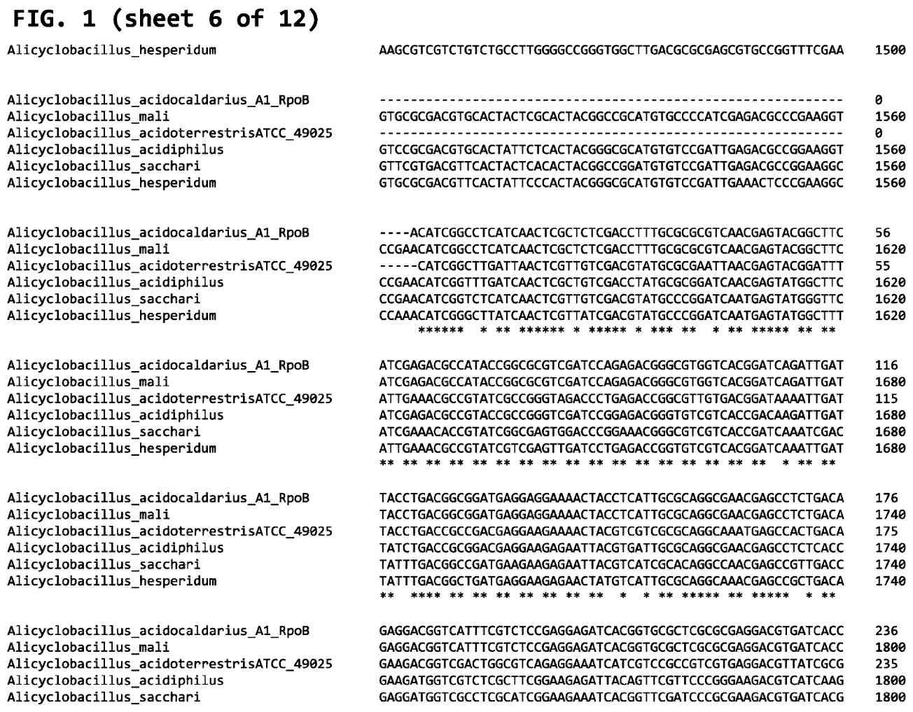

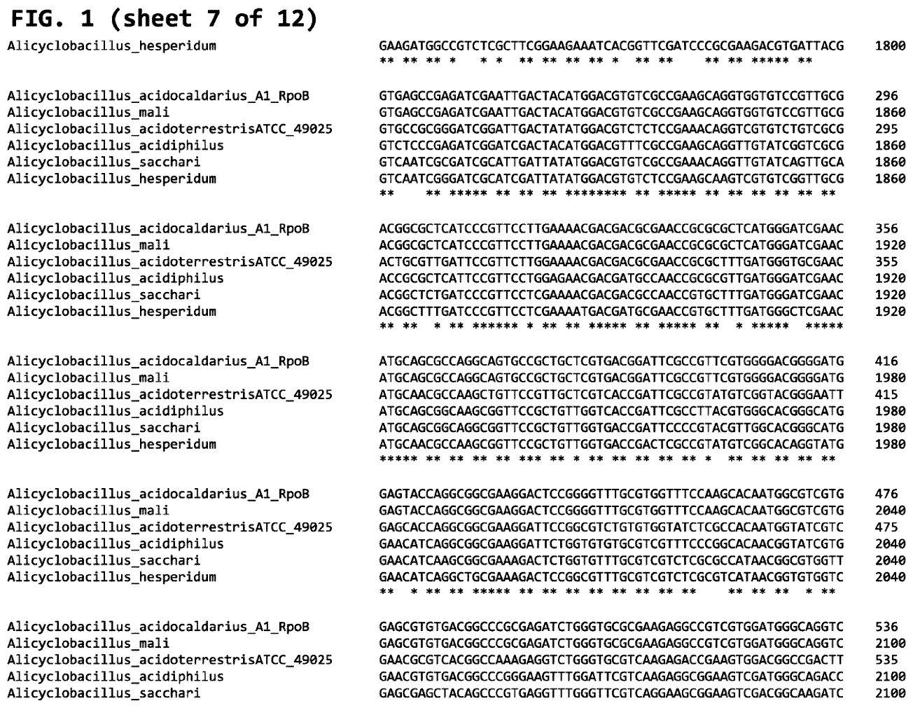

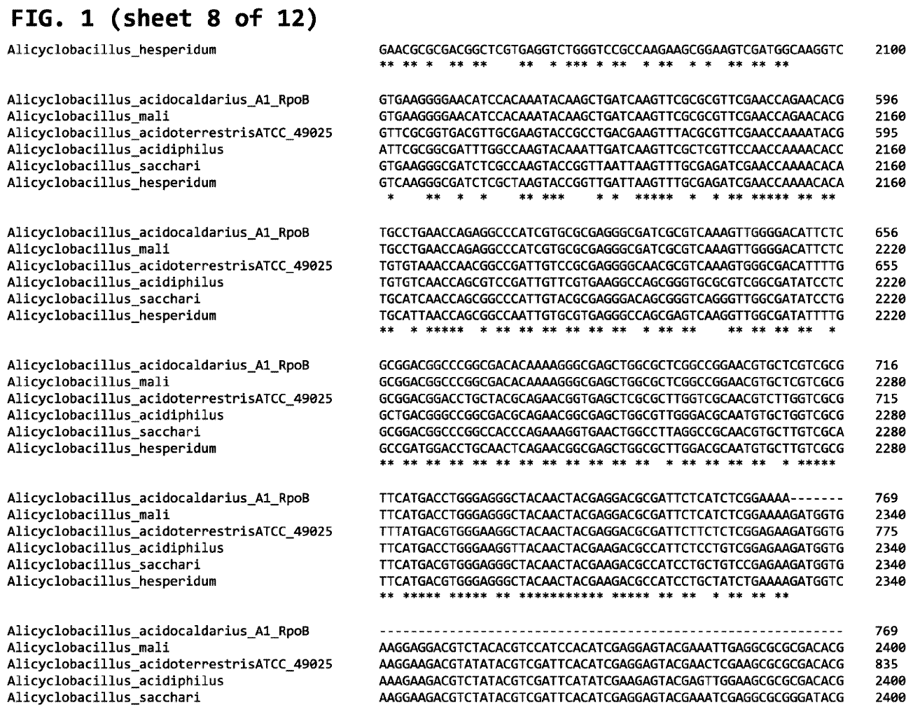

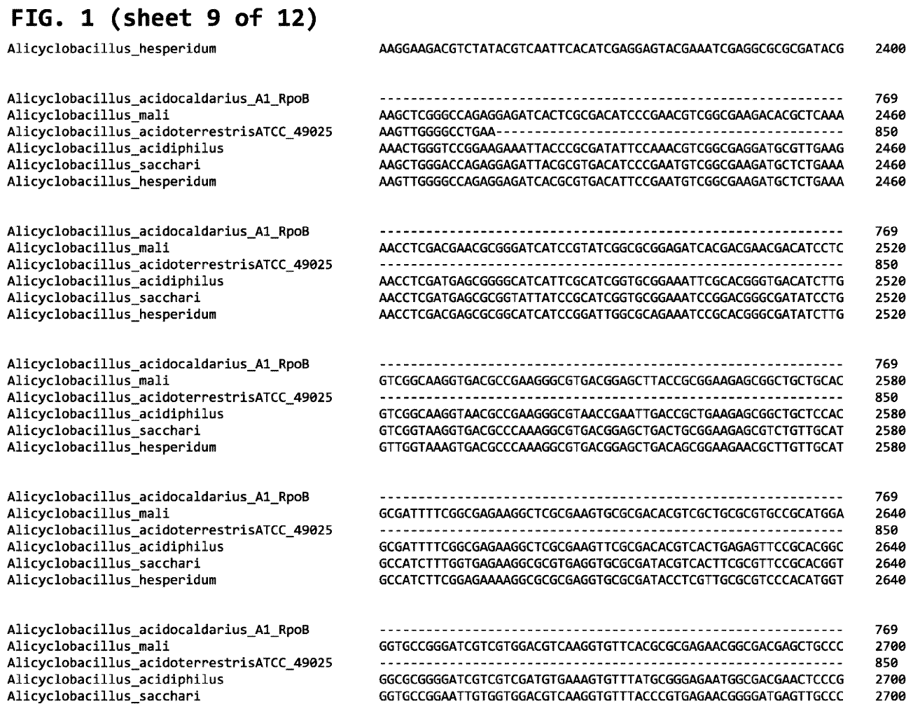

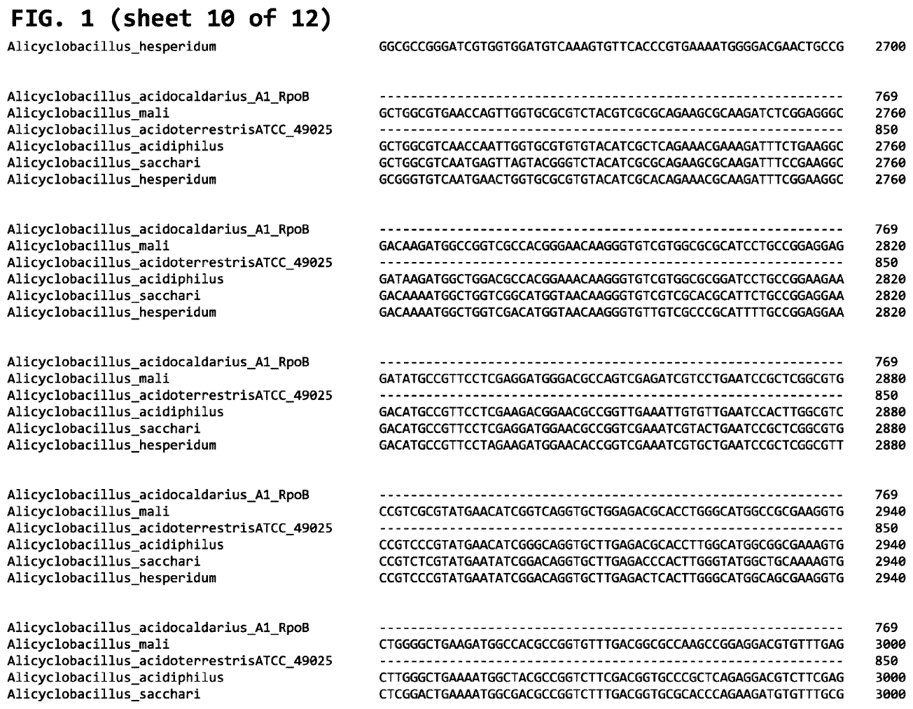

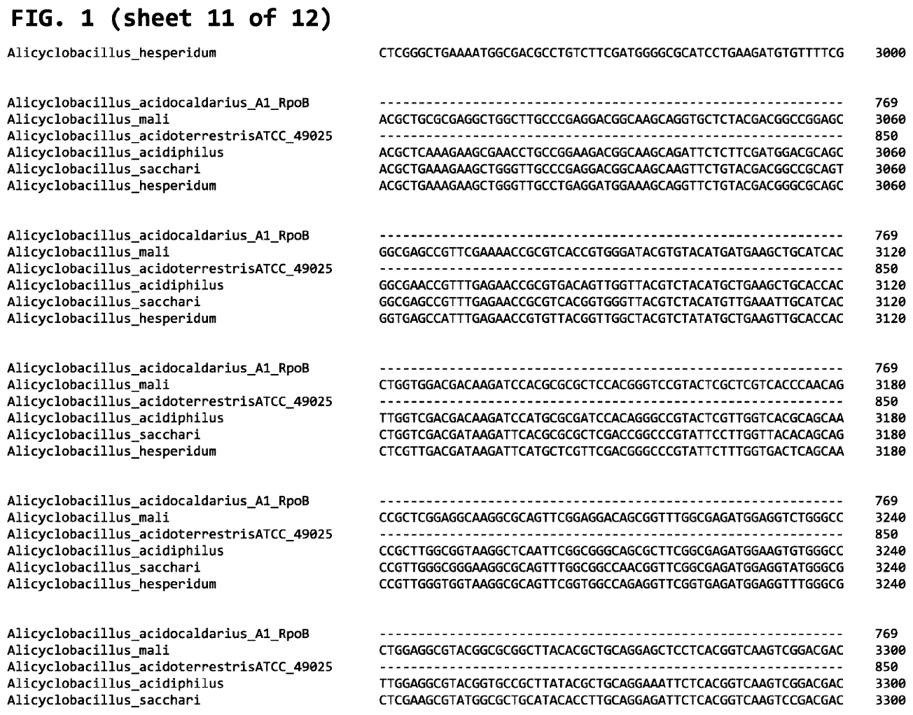

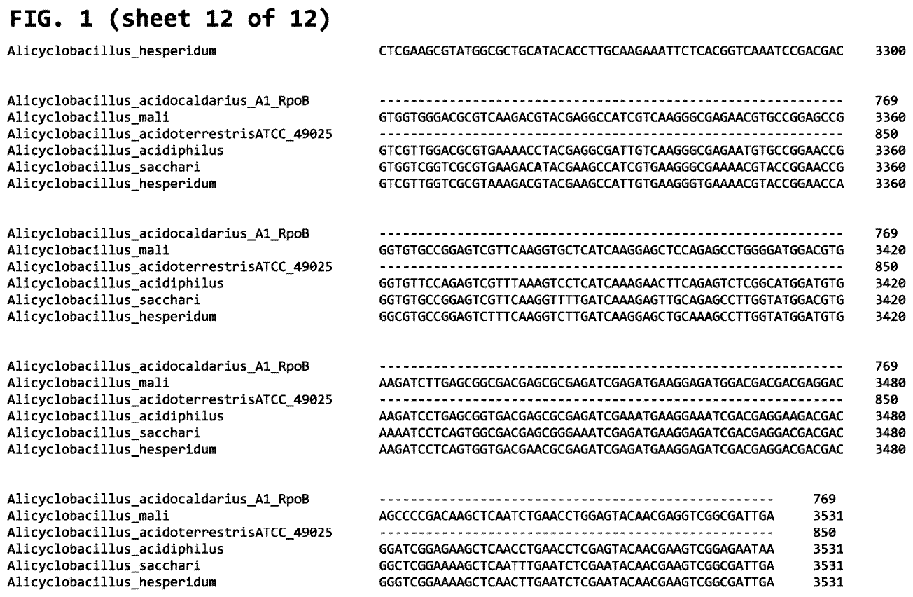

Primers are selected within the conserved regions shown in the gene sequence alignments (e.g., FIGS. 1-3) to amplify a fragment with proper size for optimal detection. One primer is located at each end of the sequence to be amplified. Such primers will normally be between 10 to 35 nucleotides in length and have a preferred length from between 18 to 22 nucleotides. The smallest sequence that can be amplified is approximately 50 nucleotides in length (e.g., a forward and reverse primer, both of 20 nucleotides in length, whose location in the sequences is separated by at least 10 nucleotides). Much longer sequences can be amplified. Preferably, the length of sequence amplified is between 75 and 250 nucleotides in length, and between 75 and 150 for PCR assay.

One type of primer used herein is the “forward primer” and is located at the left end of the region to be amplified. The forward primer is identical in sequence to a region in the top strand of the DNA (when a double-stranded DNA is pictured using the convention where the top strand is shown with polarity in the 5′ 10 3′ direction). The sequence of the forward primer is such that it hybridizes to the strand of the DNA which is complementary to the top strand of DNA.

Another type of primer used herein is the “reverse primer” and is located at the right end of the region to be amplified. The sequence of the reverse primer is such that it is complementary in sequence to, i.e., it is the reverse complement of a sequence in, a region in the top strand of the DNA. The reverse primer hybridizes to the top strand of the DNA.

PCR primers should also be chosen subject to a number of other conditions. In some embodiments, PCR primers used herein are 10 to 30 nucleotides in length to minimize hybridization to greater than one region in the template. In some embodiments, primers with long runs of a single base are avoided. In some embodiments, Primers used herein have a percent G+C content of between 40 and 60%. In embodiments, the percent G+C content of the 3′ end of the primer is higher than the percent G+C content of the 5′ end of the primer. In embodiments, primers do not contain sequences that can hybridize to another sequence within the primer. In embodiments, multiple primers used in the same PCR reaction do not hybridize to each other. Although PCR primers are preferably chosen subject to the recommendations above, it is not necessary that the primers conform to these conditions. Other primers may work, but may have a lower chance of yielding good results.

PCR primers that can be used to amplify DNA within a given sequence can be chosen using one of a number of computer programs that are available. Such programs choose primers that are optimum for amplification of a given sequence (i.e., such programs choose primers subject to the conditions stated above, plus other conditions that may maximize the functionality of PCR primers). One computer program is the Genetics Computer Group (GCG recently became Accelrys) analysis package which has a routine for selection of PCR primers. There are also several web sites that can be used to select optimal PCR primers to amplify an input sequence. One such web site is http://alces.med.umn.edu/rawprimer.html. Another such web site is http://www-genome.wi.mit.edu/cgi-bin primer/pr mer3_www.cgi.

### Alignment of Gene Sequences

Gene sequence alignment as used herein refers to the technique or process of comparing and detecting similarities between two of more sequences. Gene sequence alignment is a powerful tool to identify regions of similarity from multiple gene sequences that may be a consequence of functional relationships, and therefore is helpful to primer design. Alignment of target genes or genes of interests may be performed by any commercial or non-commercial algorithms or analysis packages. Examples of the alignment algorithms include Serial Cloner program, ClustalW, etc.

Making the Oligonucleotide Primers and/or Probes

The oligonucleotide primers and/or probes according to the present disclosure can be made in a number of ways. One way to make these oligonucleotides is to synthesize them using a commercially-available nucleic acid synthesizer. A variety of such synthesizers exists and is well known to those skilled in the art. Many such synthesizers use phosphoramidite chemistry, although other chemistries can be used. Phosphoramidite chemistry utilizes DNA phosphoramidite nucleosides, commonly called monomers, to synthesize the DNA chain or oligonucleotide. Such monomers are modified with a dimethoxytrityl (DMT) protecting group on the 5′-end, a b-cyanoethyl protected 3′-phosphite group, and may also include additional modifiers that serve to protect reactive primary amines in the heterocyclic ring structure (to prevent branching or other undesirable side reactions from occurring during synthesis).

To make an oligonucleotide of a specific sequence, phosphoramidite nucleosides are added one-by-one in the 3′-5′ direction of the oligonucleotide, starting with a column containing the 3′ nucleoside temporarily immobilized on a solid support. Synthesis initiates with cleavage of the 5′-trityl group of the immobilized 3′ nucleoside by brief treatment with acid [dichloroacetic acid (DCA) or trichloroacetic acid (TCA) in dichloromethane (DCM)] to yield a reactive 5′-hydroxyl group. The next monomer, activated by tetrazole, is coupled to the available 5′-hydroxyl and the resulting phosphite linkage is oxidized to phosphate by treatment with iodine (in a THE/pyridine/H₂O solution). The above describes the addition of one base to the oligonucleotide. Additional cycles are performed for each base that is added. The final oligonucleotide added does not have a 5′ phosphate. When synthesis is complete, the oligonucleotide is released from the support by ammonium hydroxide and deprotected (removal of blocking groups on nucleotides). Normally, oligonucleotides of up to 150-180 bases long can be efficiently synthesized by this method using a nucleic acid synthesizer. To make oligonucleotide that are longer than 100 bases, two single-stranded oligonucleotides, that are partially complementary along their length, can be synthesized, annealed to one another to form a duplex, and then ligated into a plasmid vector. Once a plasmid containing the ligated duplexes has been formed, additional oligonucleotide duplexes can be ligated into the plasmid, adjacent to the previously ligated duplexes, to form longer sequences. It is also possible to sequentially ligate oligonucleotide duplexes to each other, to form a long, specific sequence, and then clone the single long sequence into a plasmid vector.

By “nucleotide sequence” or “nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. It is generally preferred that the present nucleic acid does not comprise any insertions, deletions, inversions, and/or substitutions. However, it may be suitable in some instances, as discussed herein, for the nucleic acid to comprise one or more insertions, deletions, inversions, and/or substitutions.

The present nucleic acids can be constructed based on chemical synthesis (as discussed above) and/or enzymatic ligation reactions using procedures known in the art. See, for instance, Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, NY, 1994. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 54 carboxyhydroxymethyl) uracil, 5-carboxymethylaminomethyl 1-2-thiouridine, 5-carboxymethylaminomethyluracil, dihydrouracil, beta-D-galactosylqueosine, inosine, N⁶-isopentenyladenine, 1-methylguanine, l-methylinosine, 2,2-dimethylguanine, 2-methyladenine, 2-methylguanine, 3-methyl cytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine.

Isolation of DNA from Samples

DNA is isolated or extracted from the microorganism cells contained within the test sample. For example, DNA extraction may be performed using any of numerous commercially available kits for such purpose. One such kit, Sigma Aldrich Genomic DNA extraction kit, is available from Sigma Aldrich, St. Louis. The DNA extraction may contain paper filters onto which cells are applied. Through treatment of the paper filters as described by the manufacturer, most cellular components remain in the paper filter and DNA is released into an aqueous solution. The DNA in the solution can then be added to various enzymatic amplification reactions, as are discussed below. Other commercially available kits exist for extraction of DNA from cells. Commercial kits do not have to be used, however, in order to obtain satisfactory DNA. Standard methods, well known to those skilled in the art, have been published in the scientific literature. Such methods commonly involve lysis of cells and removal of cellular components other than nucleic acids by precipitation or by extraction with organic solvents. Enzymatic treatment with proteases and ribonucleases can be used to remove proteins and RNA, respectively. DNA is then commonly precipitated from the sample using alcohol.

### PCR

The term “PCR” as used herein refers to the various forms of PCR known in the art including, but not limited to, quantitative PCR, reverse-transcriptase PCR, real-time PCR, hot start PCR, long PCR, LAPCR, multiplex PCR, touchdown PCR, and the like. A variety of methods can be used to determine if a PCR product has been produced. One way to determine if a PCR product has been produced in the reaction is to analyze a portion of the PCR reaction by agarose gel electrophoresis. For example, a horizontal agarose gel of from 0.6 to 2.0% agarose is made and a portion of the PCR reaction mixture is electrophoresed through the agarose gel. After electrophoresis, the gel is stained with ethidium bromide. PCR products are visible when the gel is viewed during illumination with ultraviolet light. By comparison to standardized size markers, it is determined if the PCR product is of the correct expected size.

The PCR procedure preferably is done in such a way that the amount of PCR products can be quantified. Such “quantitative PCR” procedures normally involve comparisons of the amount of PCR product produced in different PCR reactions. A number of such quantitative PCR procedures, and variations thereof, are well known to those skilled in the art. One inherent property of such procedures, however, is the ability to determine relative amounts of a sequence of interest within the template that is amplified in the PCR reaction.

One particular method of quantitative PCR used to quantify copy numbers of sequences within the PCR template is a modification of the standard PCR called “real-time PCR.” Real-time PCR utilizes a thermal cycler (i.e., an instrument that provides the temperature changes necessary for the PCR reaction to occur) that incorporates a fluorimeter (i.e. an instrument that measures fluorescence). In one type of real-time PCR, the reaction mixture also contains a reagent whose incorporation into a PCR product can be quantified and whose quantification is indicative of copy number of that sequence in the template.

Basic multiplex PCR approaches and the considerations necessary to perform them successfully are known in the art and are readily applied to the methods described herein, in which the ability to efficiently separate and detect amplicons of differing sires from different known targets permits the detection of multi pie (e g., 2, 3, 5, 10, 20, 50 or more) target signals in a single reaction. Multiplex PCR generally requires that interactions between primers specific for different targets be minimized in order to reduce artifacts—that is, one seeks to avoid the ability of any two primers being used in a reaction to hybridize to each other, instead of to their respective target molecules. Commonly available software packages permit the analysis and prediction of primer-primer interactions for a given set of primers.

In some aspects, the present disclosure provides a method for detecting a spoilage microorganism in a sample, the method comprising: (a) contacting a set of oligonucleotides with the sample, wherein the set of oligonucleotides comprising: a first collection of primers targeted for nucleic acids of a guaiacol-producing microorganism, and a second collection of primers targeted for nucleic acids of Alicyclobacillus acidoterrestris, (b) amplifying DNA in the sample with the said set of oligonucleotides under a multiplex polymerase chain reaction (PCR); and (c) determining the presence of PCR products of step (b), wherein the presence of a PCR product in the sample is indicative of contamination of the sample by both guaiacol-producing microorganism and Alicyclobacillus acidoterrestris.

In some embodiments, the set of oligonucleotides further comprising a third collection of primers targeted for nucleic acids of the 16 S rRNA gene common to the genus Alicyclobacillus, and wherein the presence of the PCR product in the sample is indicative of contamination of the sample by a guaiacol-producing microorganism, the specific species Alicyclobacillus acidoterrestris, and the genus Alicyclobacillus.

In some embodiments, the guaiacol-producing gene encodes vanillin decarboxylase (vdc). In certain embodiments, the guaiacol-producing gene encoding vanillin decarboxylase (vdc) has at least 75% identity to the nucleic acid sequence set forth in SEQ ID NO. 51, as shown in Table 1.

TABLE 1
Target gene encoding vanillin decarboxylase (vdc).

| Targeted gene of | | |
|---|---|---|
| gene of interest | Source | SEQ ID NO |
| Vanillin | Vde region described in | SEQ ID NO. 51 |
| decarboxylase | Dekowska et al. 2018. | |
| (vdc) | Sequence of the vdc region is | |
| | obtained from Japanese Patent | |
| | Publication No. 2003000259A. | |

In some embodiments, the second collection of primers is targeted for the rpoB gene and/or the gyrB gene of Alicyclobacillus acidoterrestris. In certain embodiments, the rpoB gene comprises a nucleic acid sequence that has at least 75% identity to any one of the nucleic acid sequences set forth in SEQ ID NOS. 9-14, as shown in Table 2. In certain embodiments, the gyrB gene comprises a nucleic acid sequence that has at least 75% identity to any one of the nucleic acid sequences set forth in SEQ ID NOS. 15-26, as shown in Table 2.

TABLE 2
The rpoB gene and/or the gyrB gene of
Alicyclobacillus acidoterrestris.

| | SEQ |
|---|---|
| Targeted gene of gene of interest | ID NO |
| rpoB gene of Alicyclobacillus_acidocaldarius_A1_RpoB | 9 |
| rpoB gene of Alicyclobacillus_mali | 10 |
| rpoB gene of Alicyclobacillus_acidoterrestrisATCC_49025 | 11 |
| rpoB gene of Alicyclobacillus_acidiphilus | 12 |
| rpoB gene of Alicyclobacillus_sacchari | 13 |
| rpoB gene of Alicyclobacillus_hesperidum | 14 |
| gyrB gene of A. contaminans_ACON_DSM17975 | 15 |
| gyrB gene of A. acidoterrestris_AT_NBRC106287 | 16 |
| gyrB gene of A. acidoterrestris_AT_ATCC49025 | 17 |
| gyrB gene of A. acidoterrestris_AT_ATCC49025_2 | 18 |
| gyrB gene of A. acidoterrestris_AT_OR3 | 19 |
| gyrB gene of A. hesperidum_AH_DSM12489 | 20 |
| gyrB gene of A. sacchari_ASach_DSM17974 | 21 |
| gyrB gene of A. acidiphilus_AP_NBRC100859 | 22 |
| gyrB gene of A. acidiphilus_AP_nondefined | 23 |
| gyrB gene of A. acidocaldarius_AC_DSM446 | 24 |
| gyrB gene of A. sendaiensis_AS_NBRC100866 | 25 |
| gyrB gene of A. sendaiensis_AS_nondefined | 26 |

In some embodiments, the 16 S rRNA gene common to the genus Alicyclobacillus comprises a nucleic acid sequence that has at least 75% identity to the any one of the nucleic acid sequences set forth in SEQ ID NOS. 27-50, as shown in Table 3.

TABLE 3
The 16 S rRNA gene common to the genus Alicyclobacillus.

| | SEQ |
|---|---|
| Targeted gene of gene of interest | ID NO |
| 16 S rRNA of B. subtilis_168 | 27 |
| 16 S rRNA of G. stearothermophilus_R-32640 | 28 |
| 16 S rRNA of D. stibilarsenatis_MLFW-2 | 29 |
| 16 S rRNA of A. contaminans_3-A191 | 30 |
| 16 S rRNA of A. contaminanas_NBRC103102 | 31 |
| 16 S rRNA of A. sendaiensis_JCM1181 | 32 |
| 16 S rRNA of A. sendaiensis_NBRC100866 | 33 |
| 16 S rRNA of A. sendaiensis_NTAP-1 | 34 |
| 16 S rRNA of A. acidocaldarius_rittmannii_DSM11297 | 35 |
| 16 S rRNA of A. acidocaldarius_rittmannii_MR1 | 36 |
| 16 S rRNA of A. acidocaldarius_acidocaldarius_DSM446 | 37 |
| 16 S rRNA of A. acidocaldarius_104-1A | 38 |
| 16 S rRNA of A. acidocaldarius_acidocaldarius_ATCC27009 | 39 |
| 16 S rRNA of A. acidocaldarius_NBRC15652 | 40 |
| 16 S rRNA of A. acidiphilus_NBRC100859 | 41 |
| 16 S rRNA of A. acidiphilus_TA-67 | 42 |
| 16 S rRNA of A. sacchari_NBRC103105 | 43 |
| 16 S rRNA of A. sacchari_RB718 | 44 |
| 16 S rRNA of A. hesperidum_DSM12489 | 45 |
| 16 S rRNA of A. heperidum_FR-11 | 46 |
| 16 S rRNA of A. acidoterrestris_DSM3922 | 47 |
| 16 S rRNA of A. acidoterrestris_ATCC49025 | 48 |
| 16 S rRNA of A. fastidiosus_NBR103109 | 49 |
| 16 S rRNA of A. fastidiosus_S-TAB | 50 |

In some embodiments, the first collection of primers hybridizes with the nucleic acids of the guaiacol-producing gene or a fragment thereof. In some embodiments, the first collection of primers comprises at least one primer that hybridizes with the nucleic acids of the gene sequence set forth in SEQ ID NOS. 51 or a fragment thereof. In certain embodiments, the first collection of primers comprises at least one primer having at least 75% identity to the nucleic acid sequence set forth in SEQ ID NOS. 1-2, as shown in Table 4.

TABLE 4
Primers for targeting guaiacol-producing gene
(Vanillin decarboxylase (vdc)).

| Primers | | |
|---|---|---|
| (Dekowska | | |
| et al. | Sequence of primer | |
| 2018) | (5′-3′); | SEQ ID NO |
| Bur5 | CCGACGTGATGCTCAARGAGCGCA | SEQ ID NO: 1 |
| Bur6 | GTSGCRTCGAGAATCATCTTGTG | SEQ ID NO: 2 |

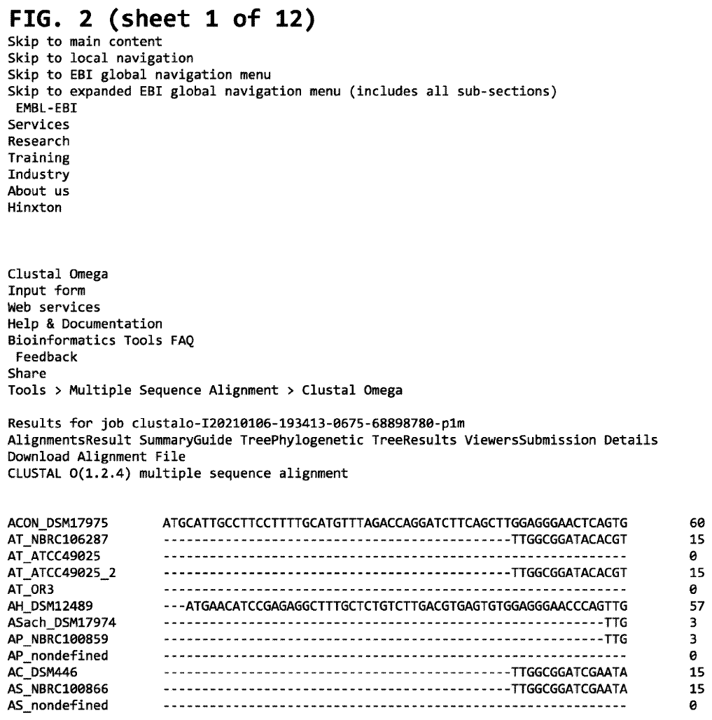

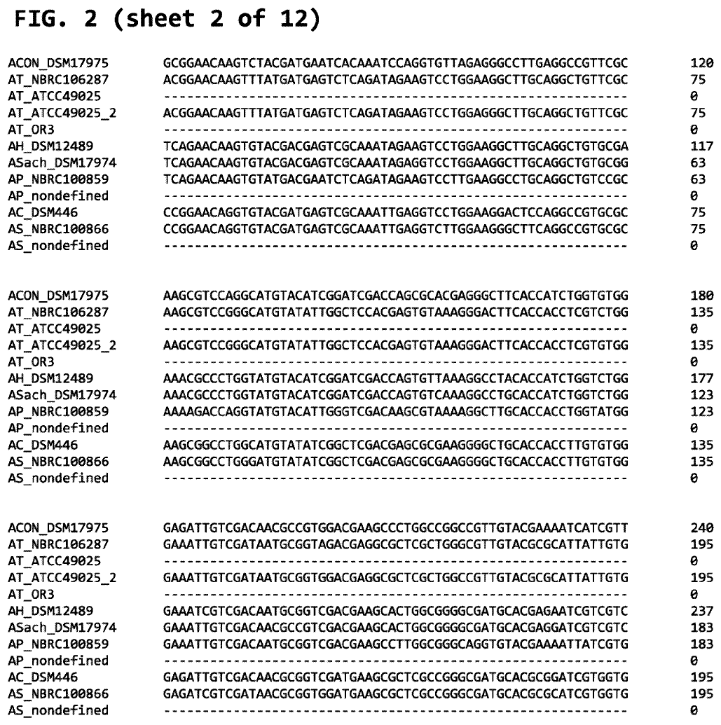

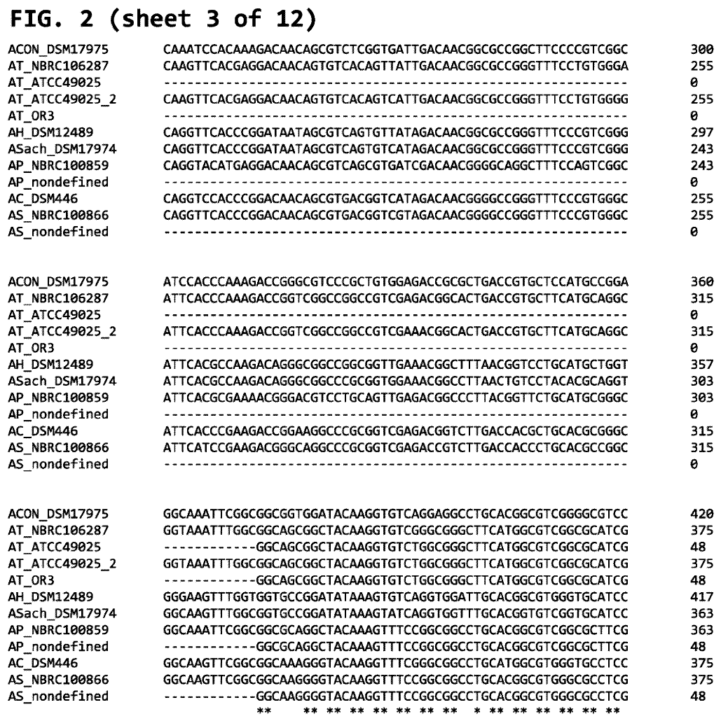

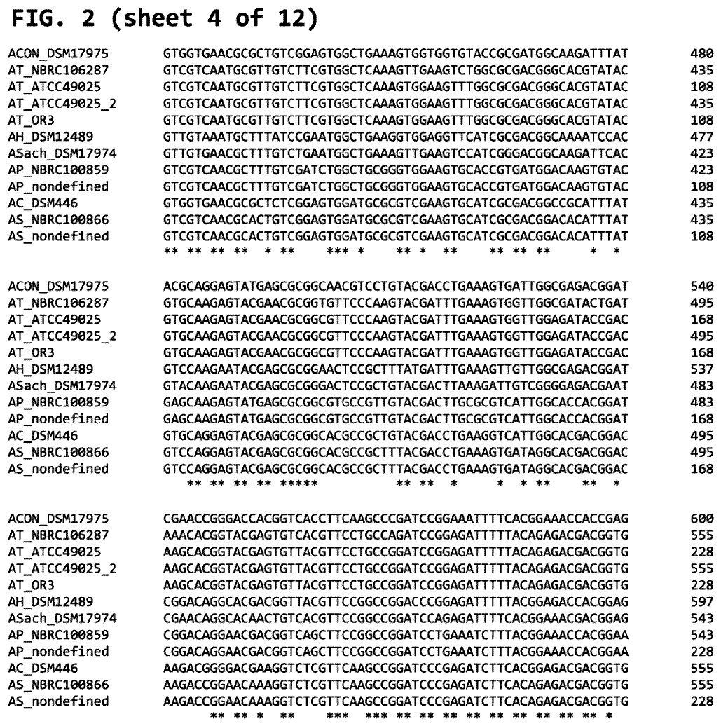

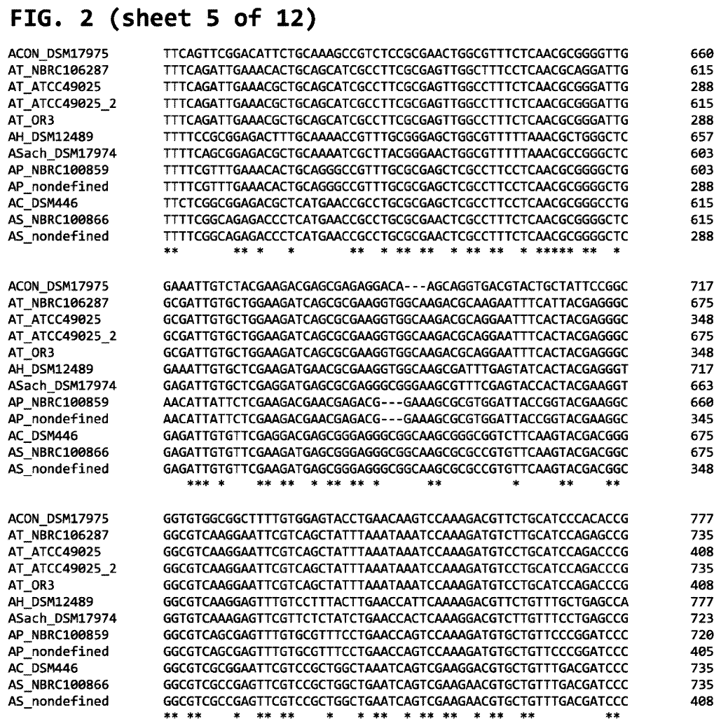

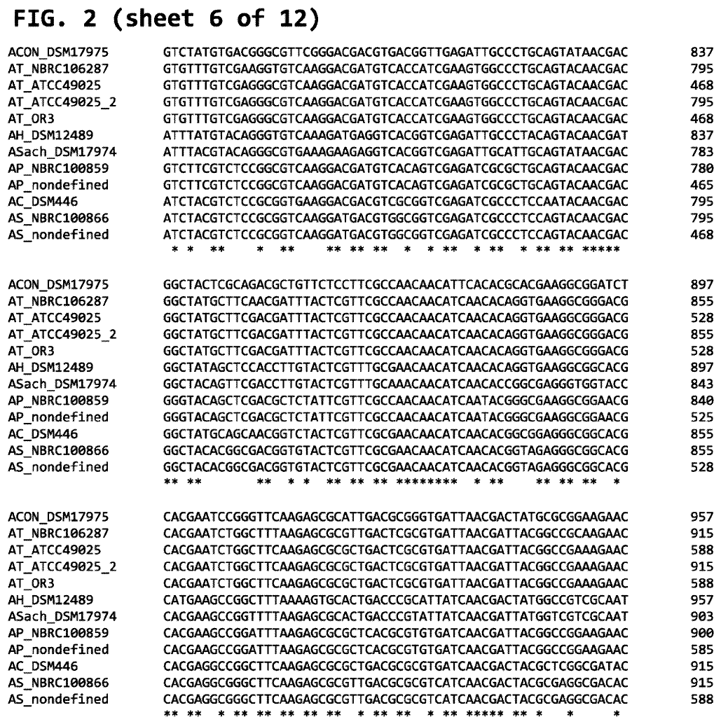

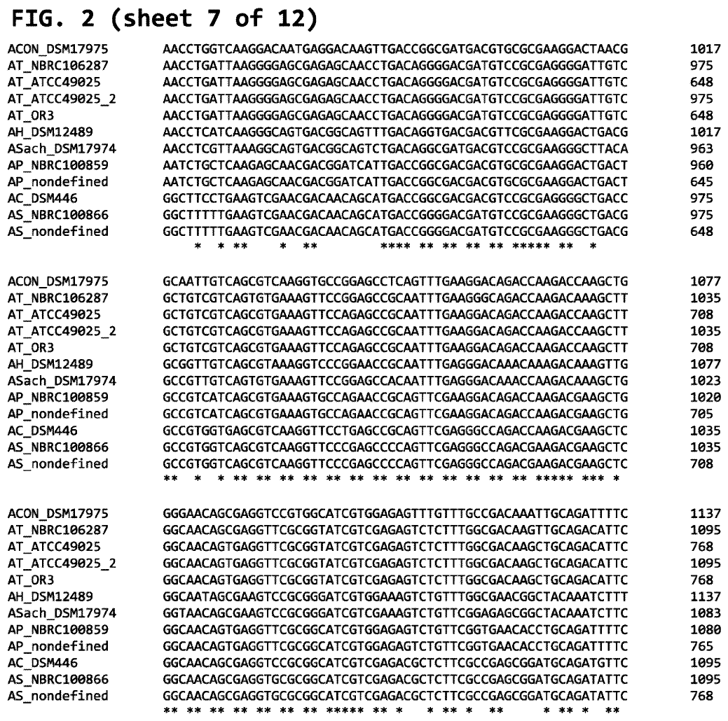

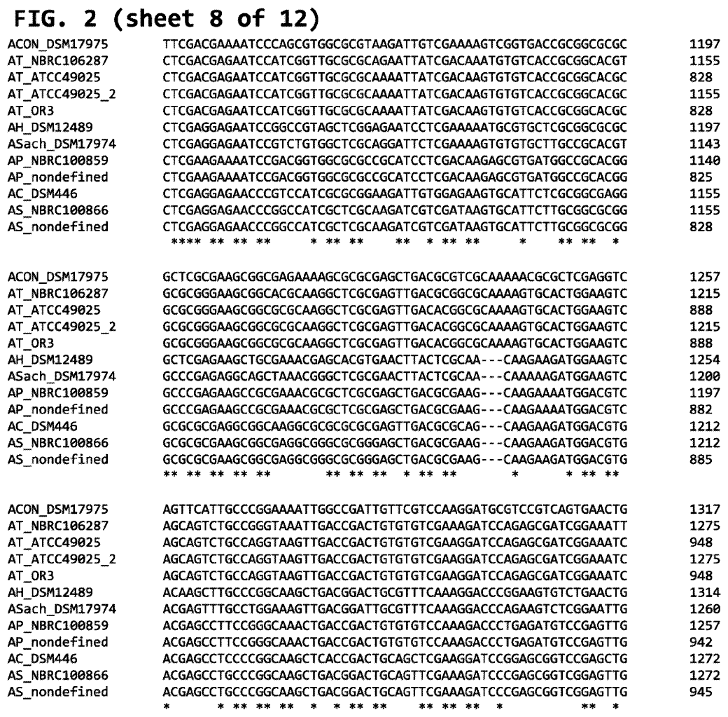

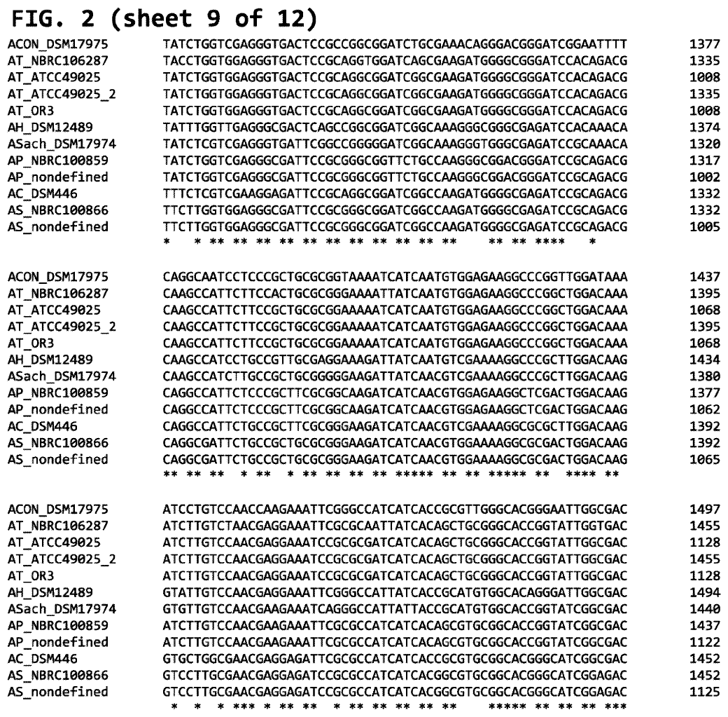

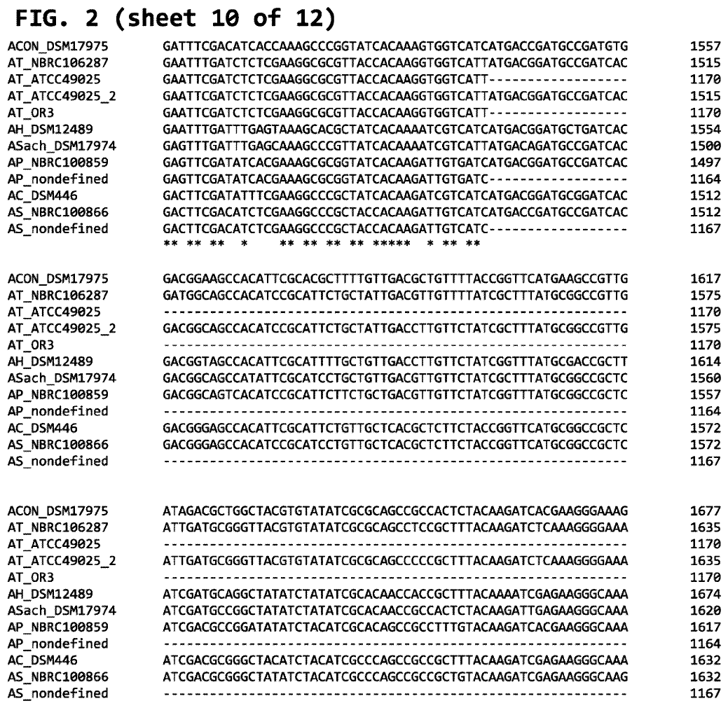

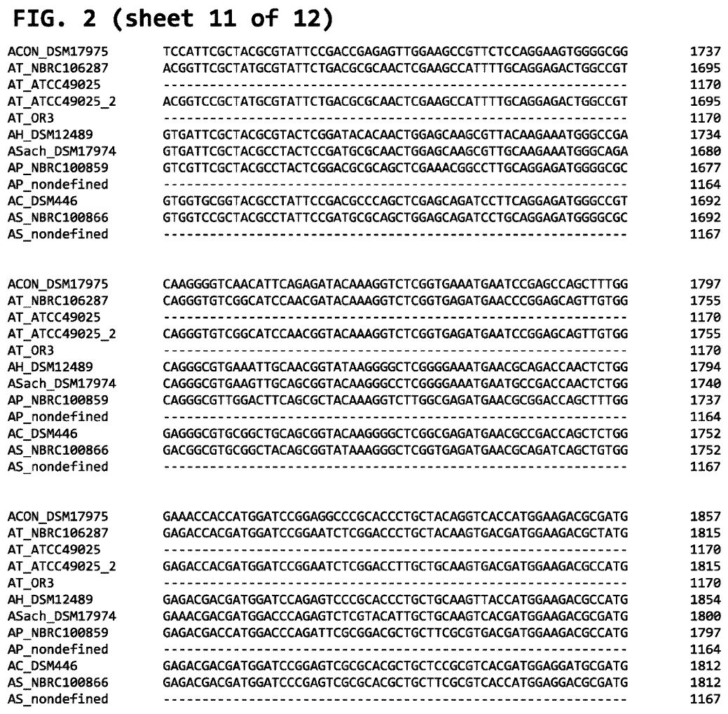

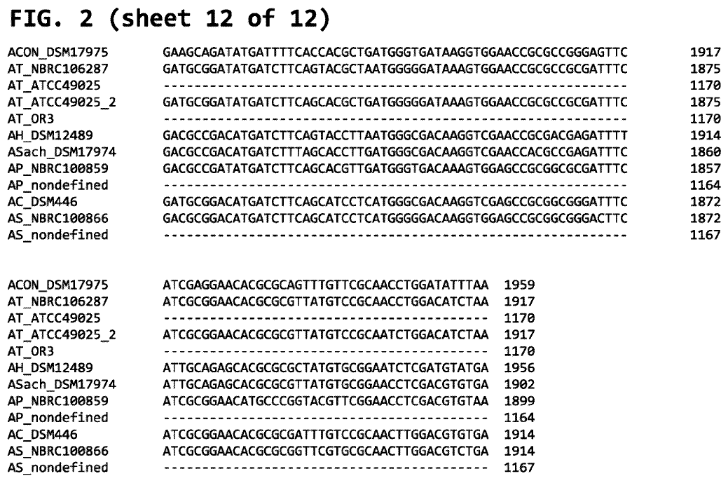

In some embodiments, the second collection of primers hybridizes with the nucleic acids of the Alicyclobacillus acidoterrestris or a fragment thereof. In some embodiments, the second collection of primers comprises at least one primer that hybridizes with the nucleic acids common to two or more gene sequences set forth in SEQ ID NOS. 9-14. In some embodiments, the second collection of primers comprises at least one primer that hybridizes with the nucleic acids common to two or more gene sequences set forth in SEQ ID NOS. 15-26. In some embodiments, the second collection of primers comprises at least one primer that hybridizes with the alignment product of gene sequences set forth in SEQ ID NOS. 9-14. In some embodiments, the second collection of primers comprises at least one primer that hybridizes with the alignment product of gene sequences set forth in SEQ ID NOS. 15-26. An example of the gene sequence alignment product of SEQ ID NOS. 9-14 is illustrated in FIG. 1. An example of the gene sequence alignment product of SEQ ID NOS. 15-26 is illustrated in FIG. 2. In certain embodiments, the second collection of primers comprises at least one primer having at least 75% identity to the nucleic acid sequence set forth in SEQ ID NOS. 3-6, as shown in Table 5.

In some embodiments, the second collection of primers comprises at least one of: a forward primer having at least 75% identity to the nucleic acid sequence set forth in SEQ ID NOS. 3 and 5. In some embodiments, the second collection of primers comprises at least one of: a reverse primer having at least 75% identity to the nucleic acid sequence set forth in SEQ ID NOS. 4 and 6.

TABLE 5
Primers for species-specific target
(Alicyclobacillus acidoterrestris).

| | Sequence of primer | |
|---|---|---|
| Primers | (5′-3′) | SEQ ID NO |
| Forward primer | CCGTCGTGAGGACGTTAT | SEQ ID NO: |
| for rpoB gene | CGCGGTGCCG | 3 |
| Reverse primer | CGCAACGTCACCGCGAAC | SEQ ID NO: |
| for rpoB gene | AAGTCGG | 4 |
| Forward primer | GTTCCCAAGTACGATTTG | SEQ ID NO: |
| for gyrB gene | AAAGTGG | 5 |
| Reverse primer | GGTTGCTCTCGCTCCCCT | SEQ ID NO: |
| for gyrB gene | TAATC | 6 |

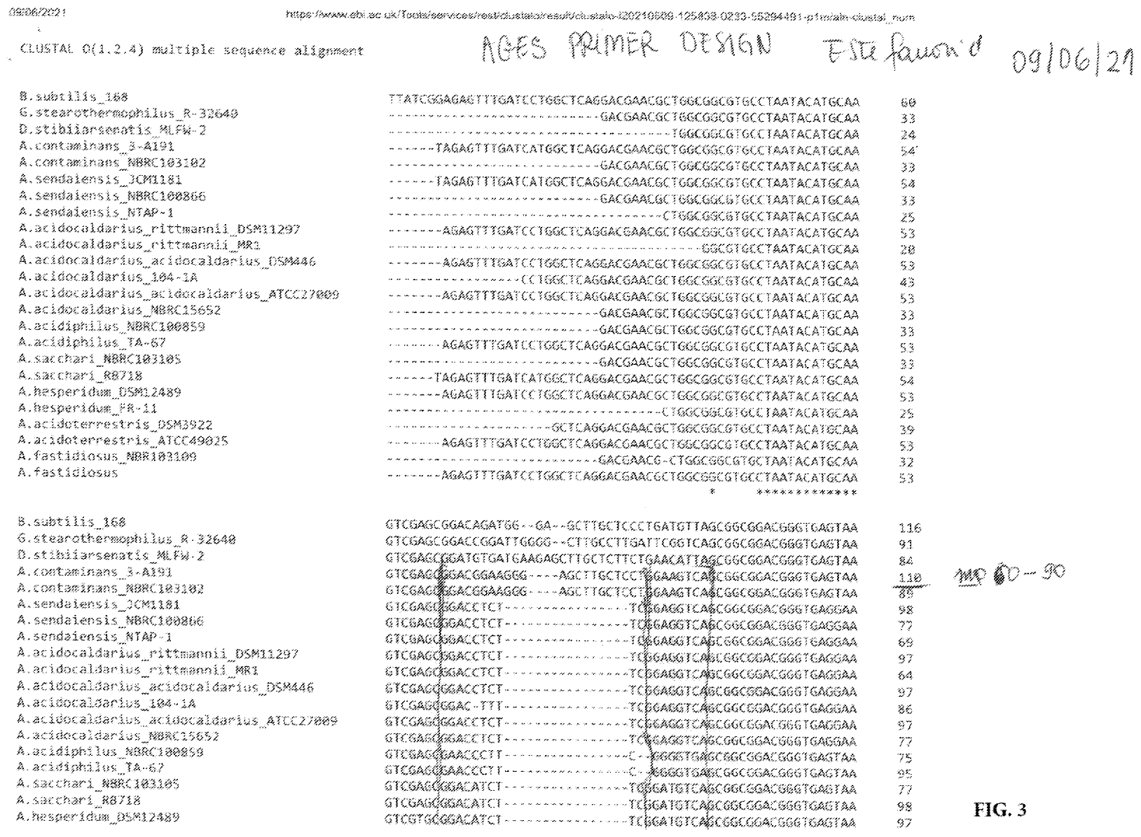

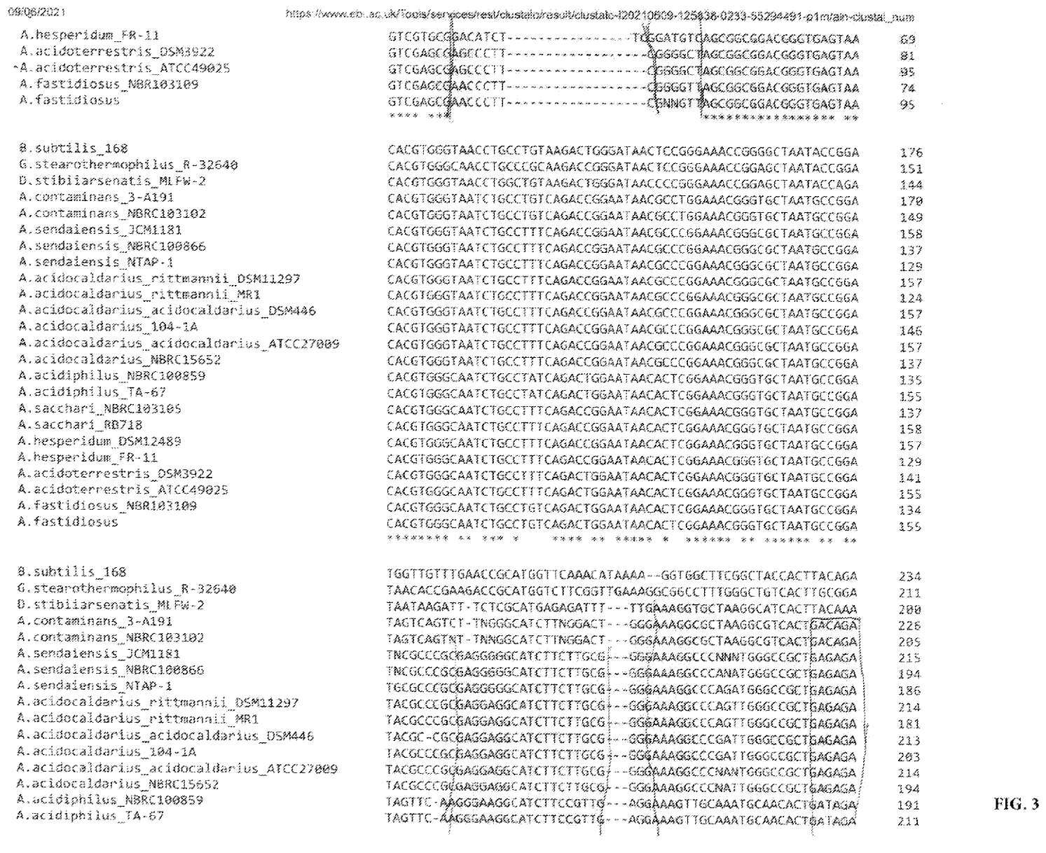

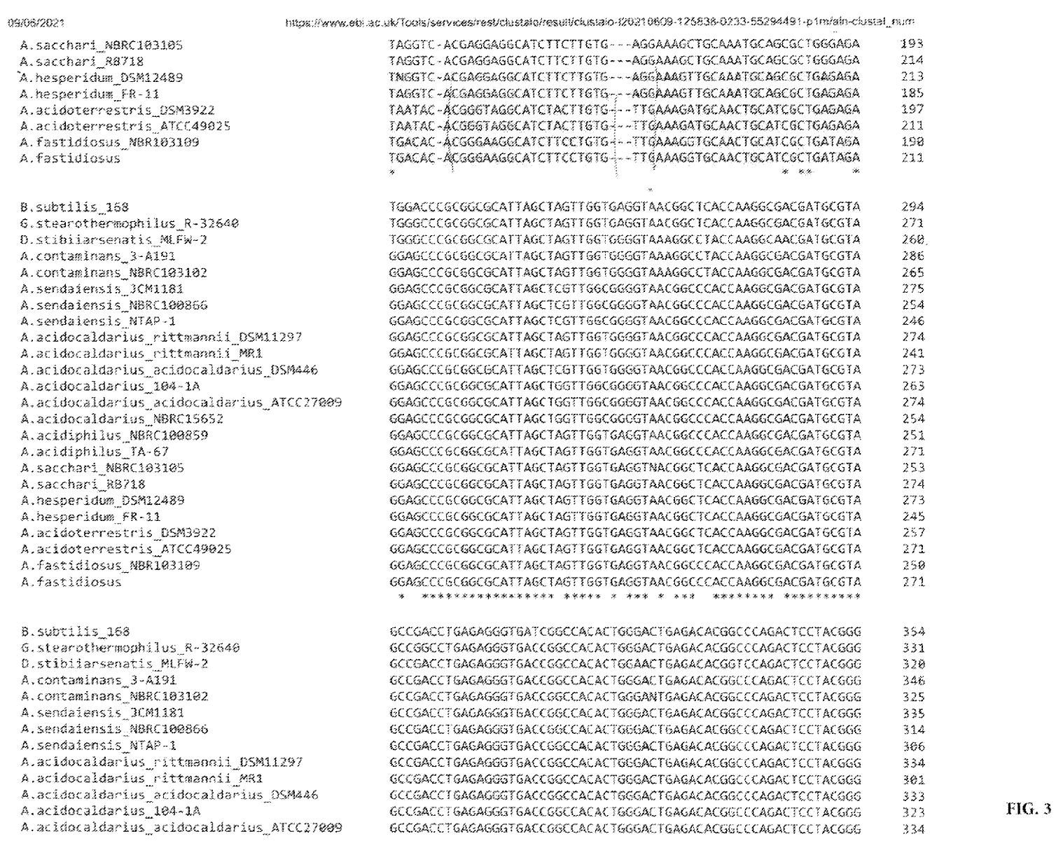

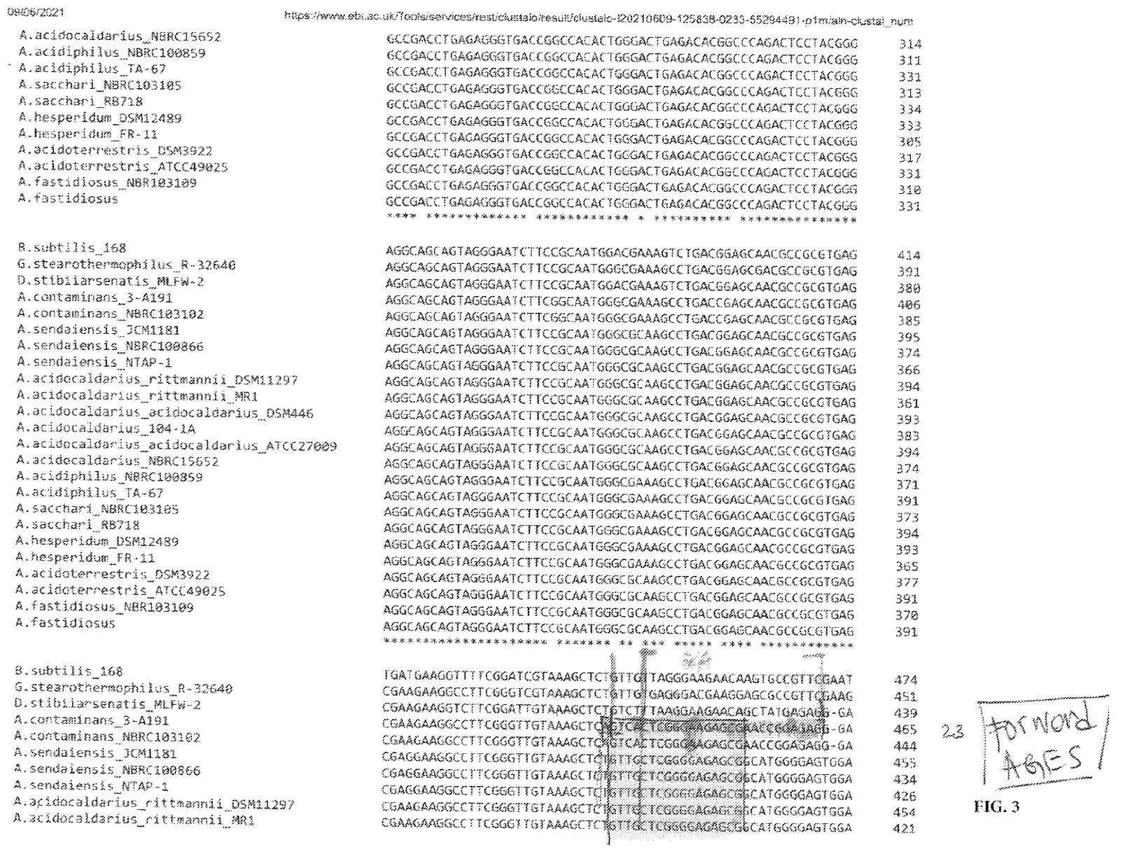

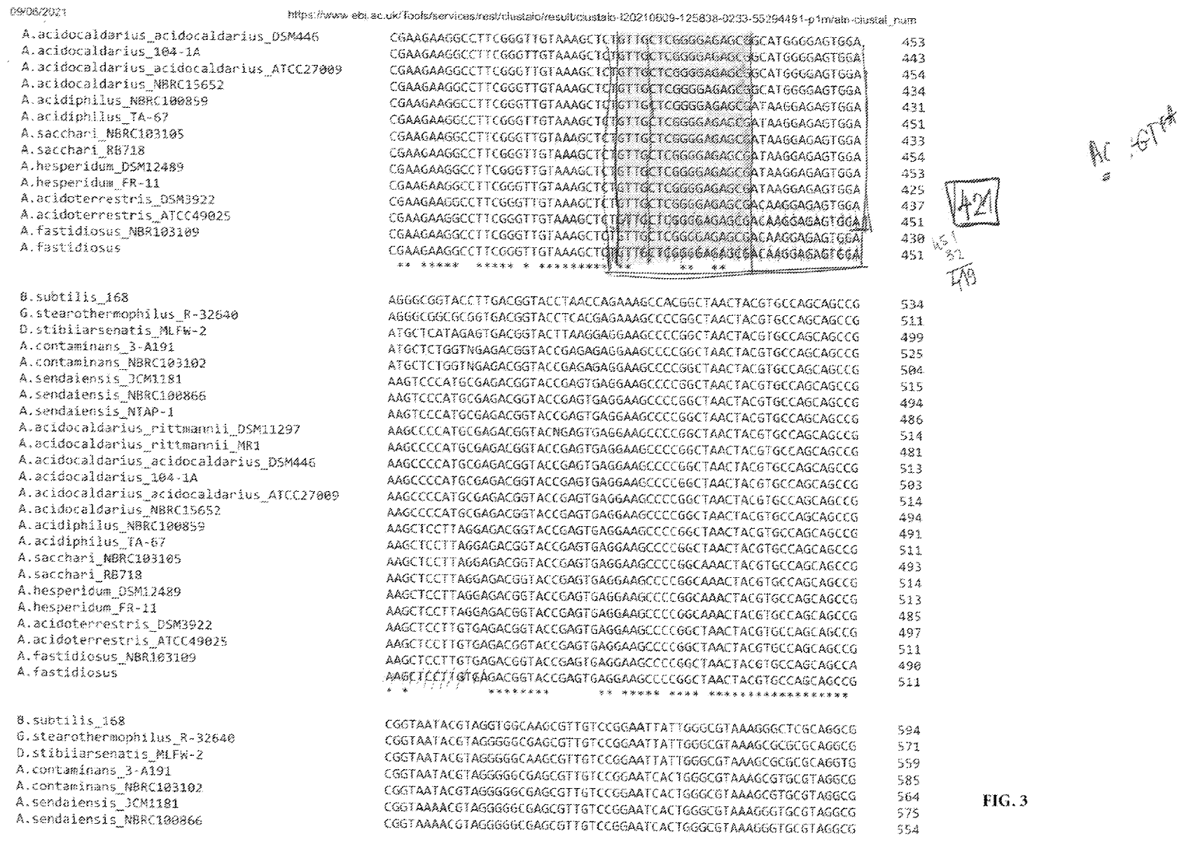

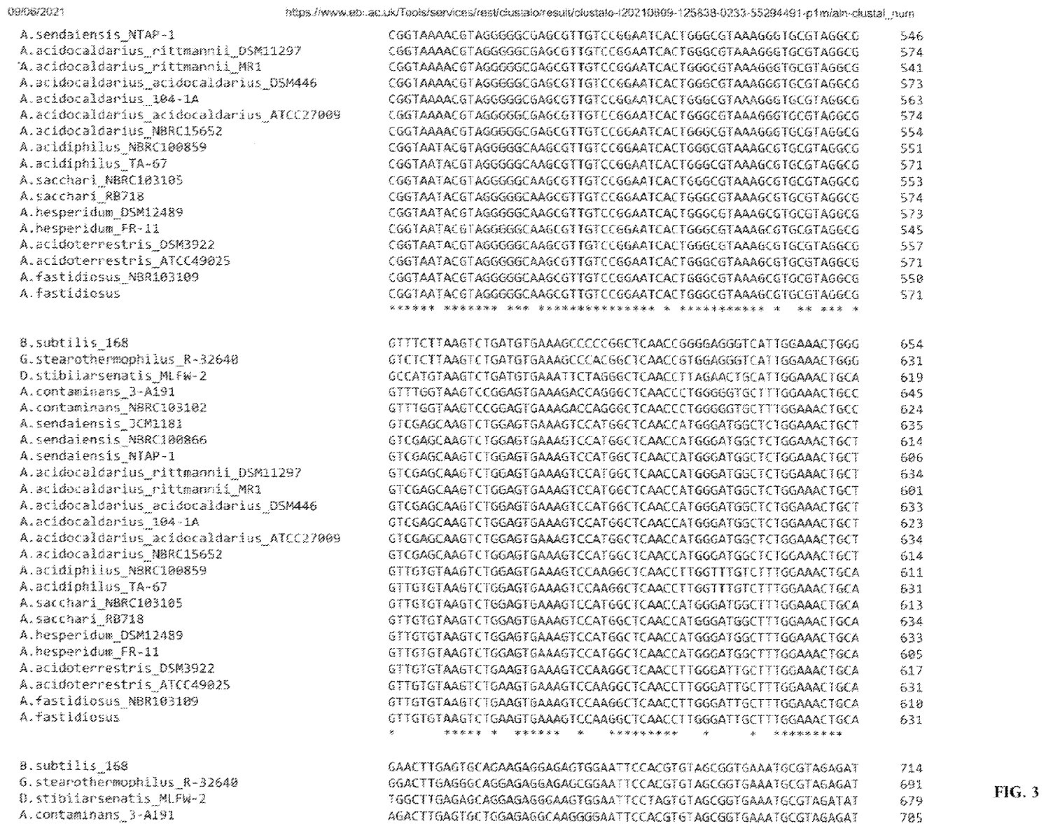

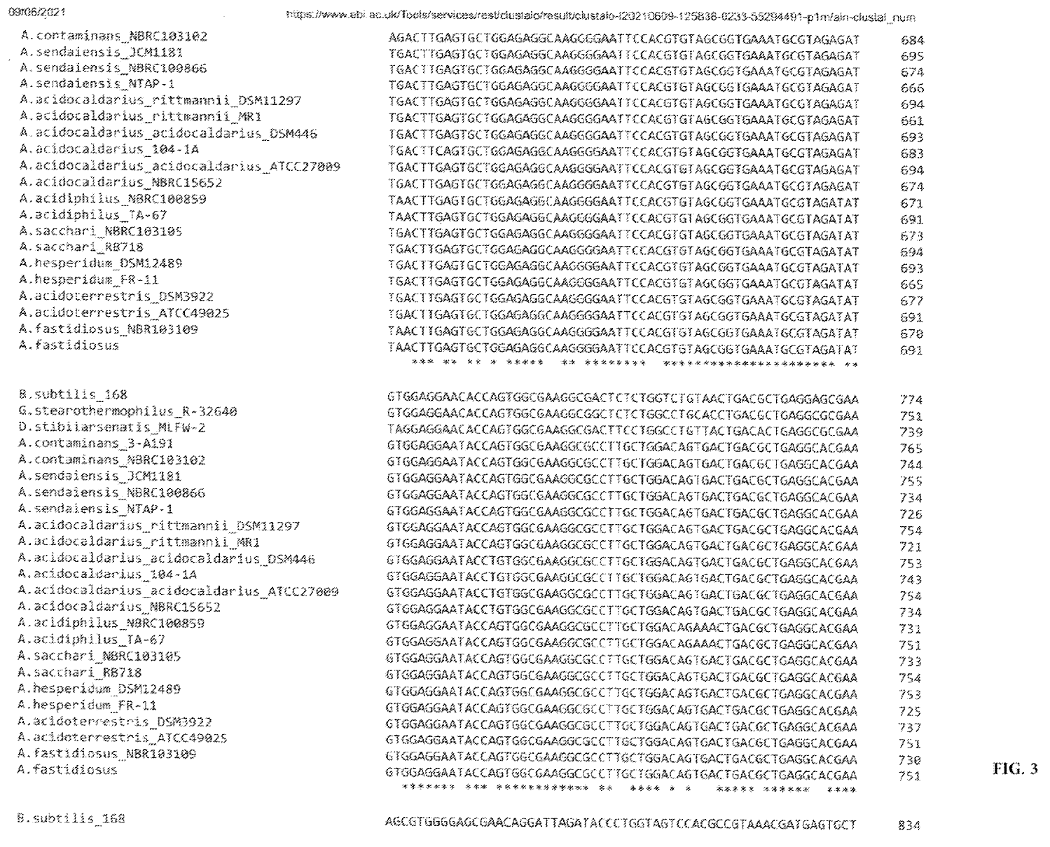

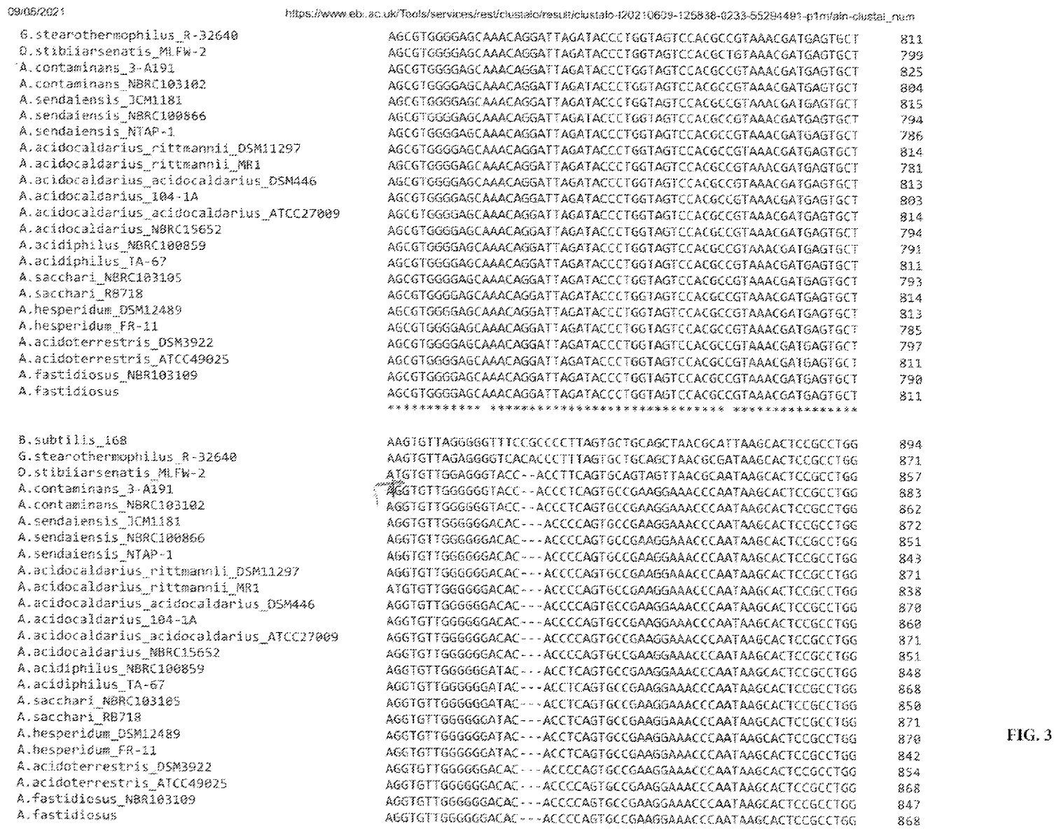

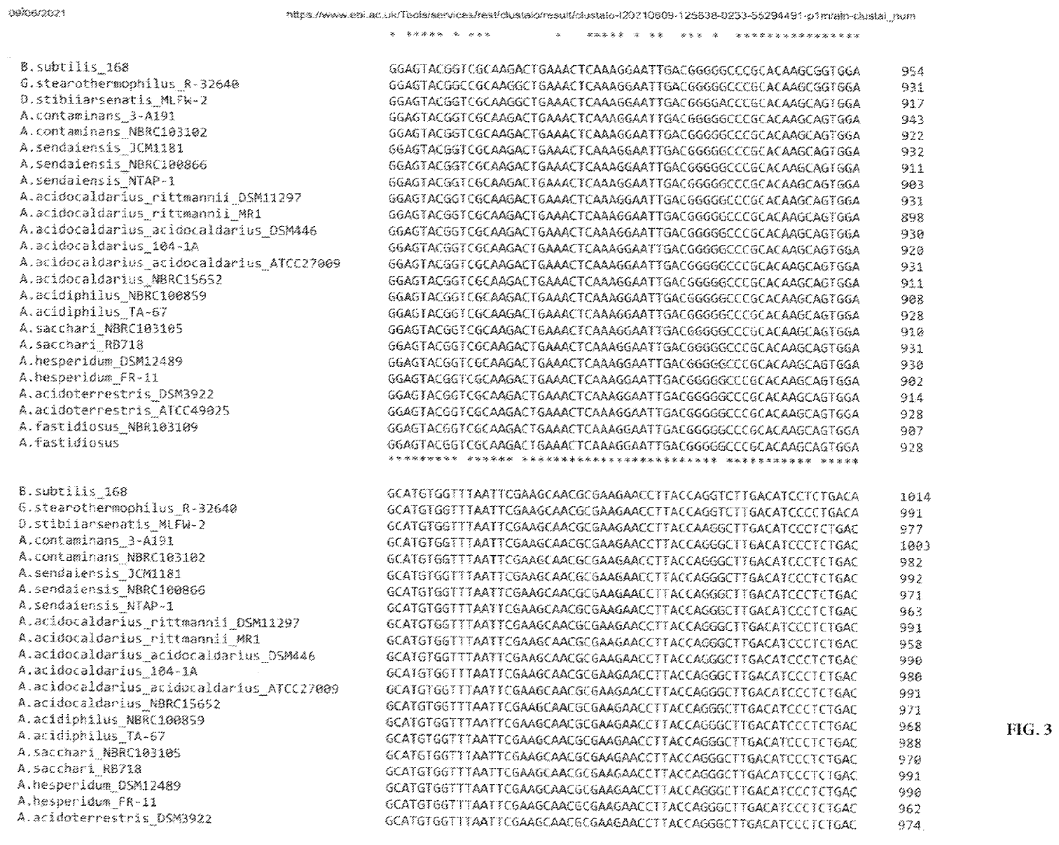

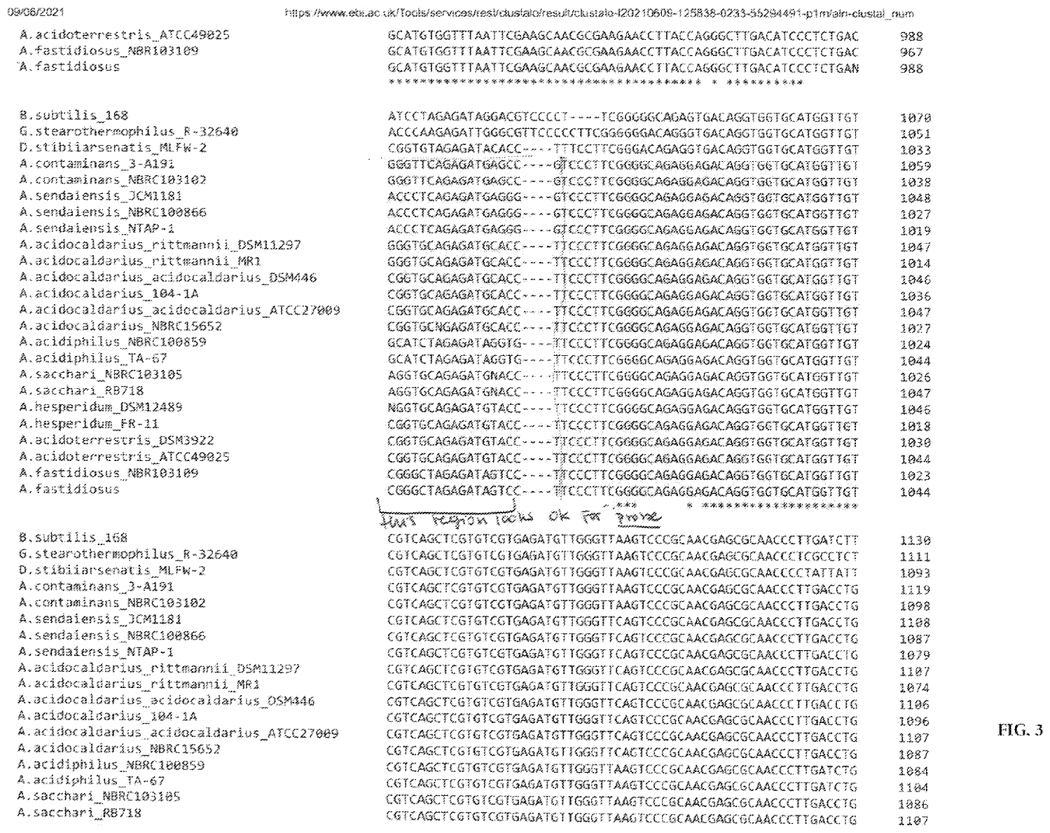

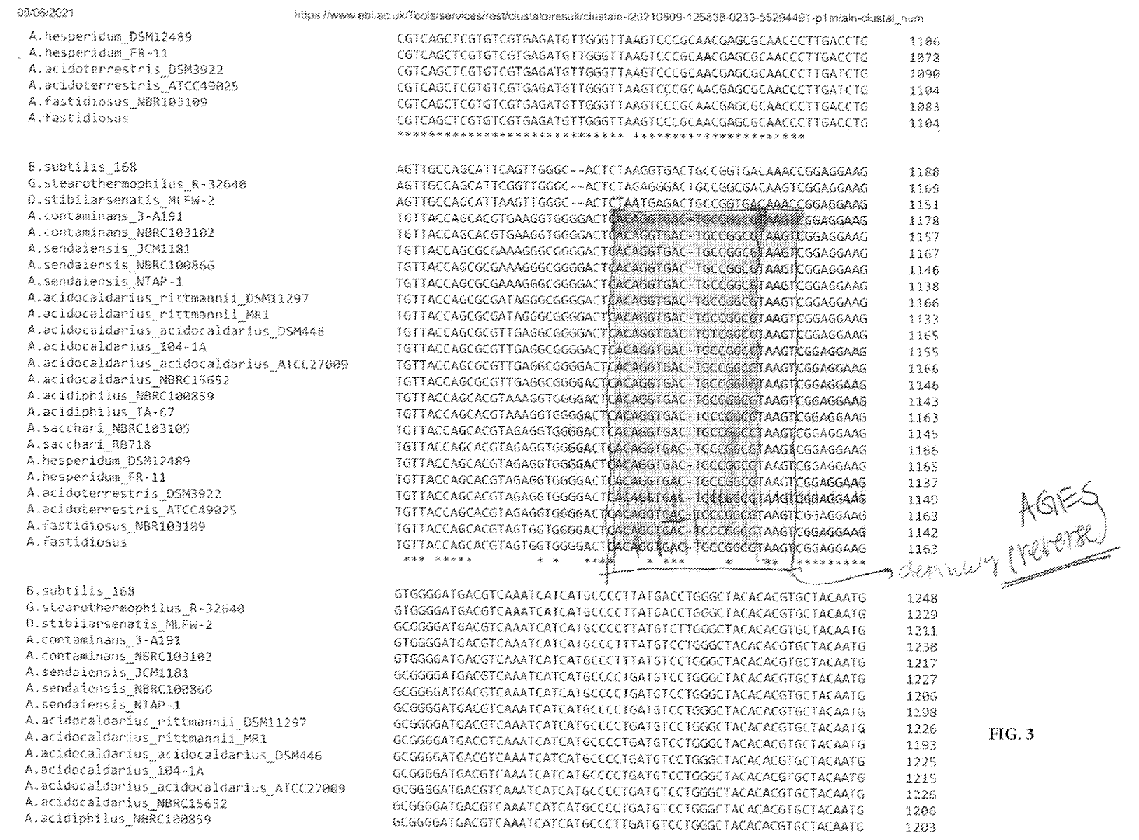

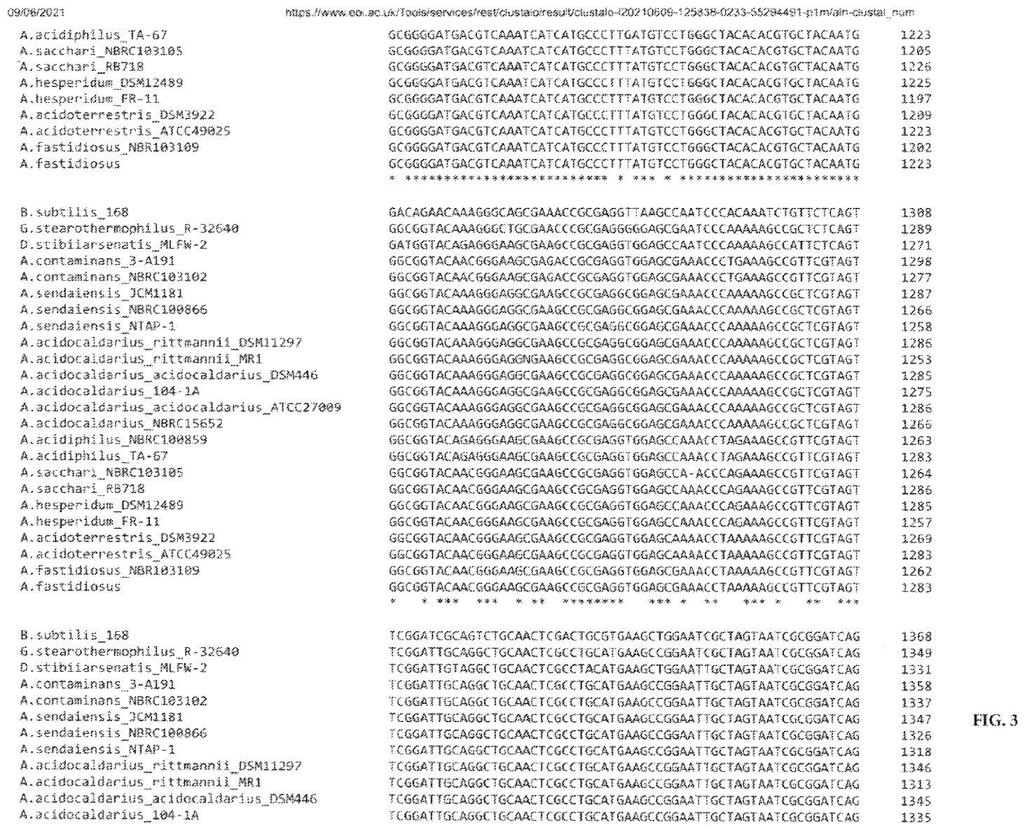

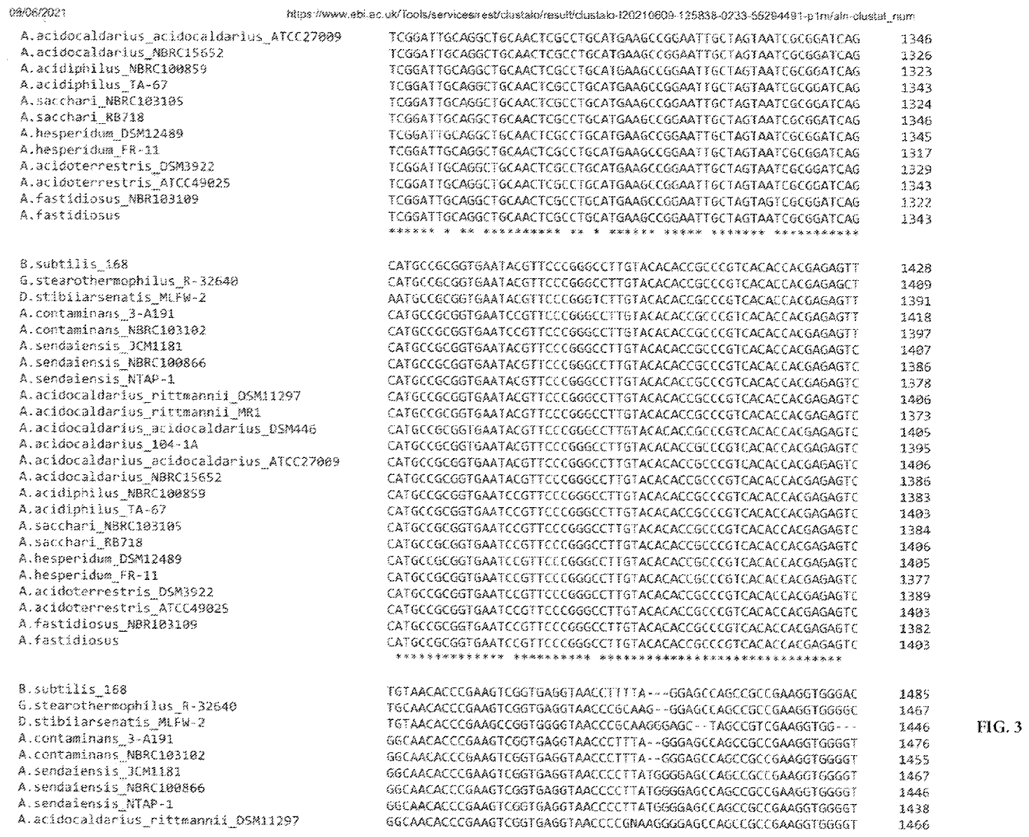

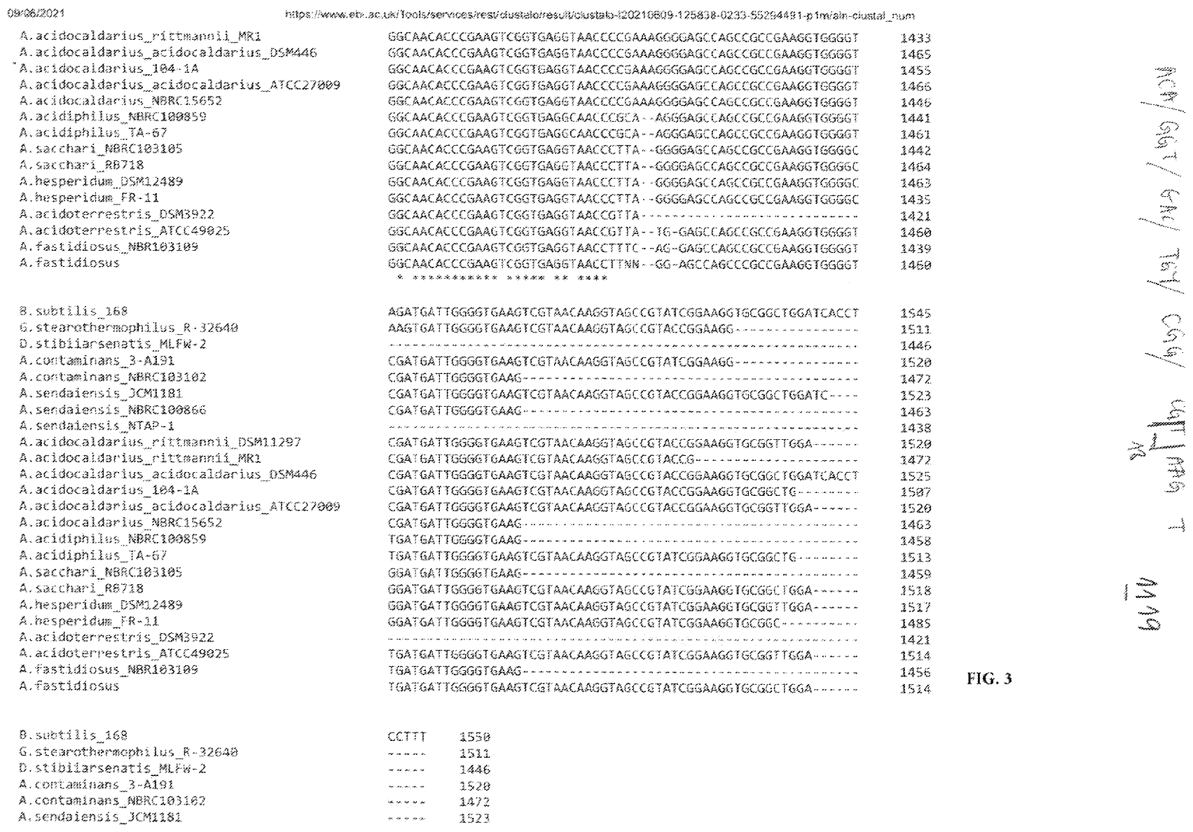

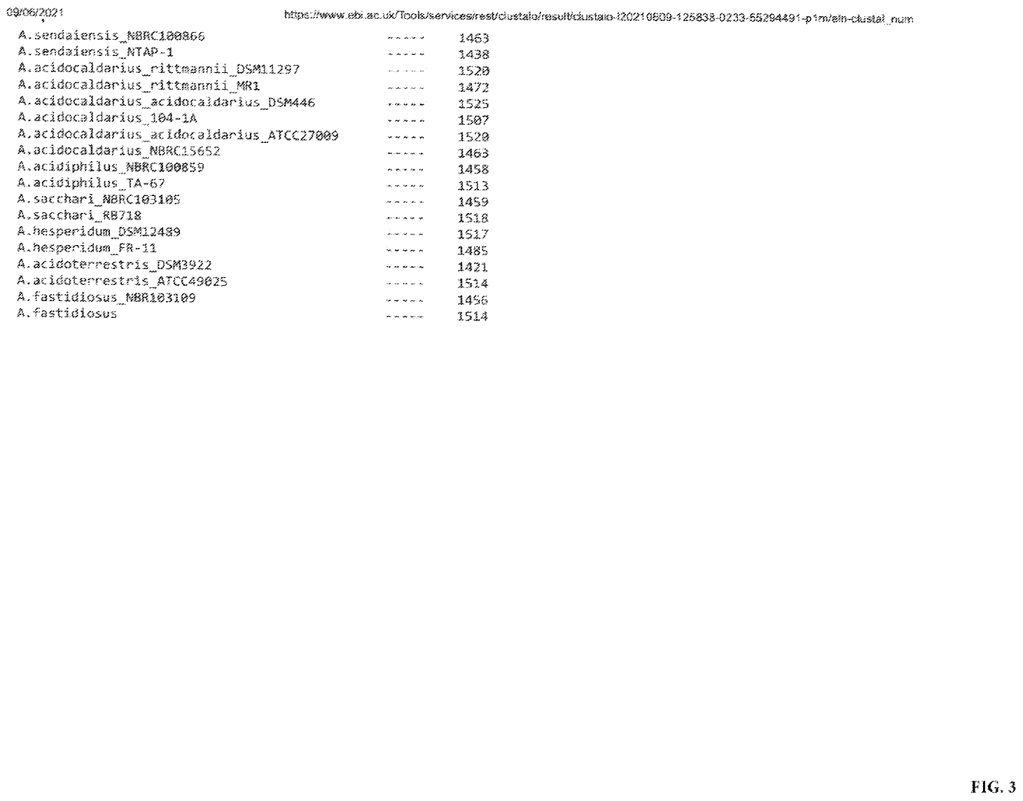

In some embodiments, the third collection of primers hybridizes with the nucleic acids of the 16 S rRNA gene. In some embodiments, the third collection of primers comprises at least one primer that hybridizes with the nucleic acids common to two or more gene sequences set forth in SEQ ID NOS. 27-50. In some embodiments, the second collection of primers comprises at least one primer that hybridizes with the alignment product of gene sequences set forth in SEQ ID NOS. 27-50. An example of the gene sequence alignment product of SEQ II) NOS. 27-50 is illustrated in FIG. 3. In certain embodiments, the third collection of primers comprises at least one primer having at least 75% identity to the nucleic acid sequence set forth in SEQ ID NOS. 7-8, as shown in Table 6.

In some embodiments, the third collection of primers comprises at least one of a forward primer having at least 75% identity to the nucleic acid sequence set forth in SEQ ID NO. 7. In some embodiments, the third collection of primers comprises at least one of a reverse primer having at least 75% identity to the nucleic acid sequence set forth in SEQ ID NO. 8.

TABLE 6
Primers for genus-specific targeting
(16 S rRNA common to genus
Alicyclobacillus, Bacillus,
Desulfuribacillus, and Geobacillus).

| | Sequence of primer | |
|---|---|---|
| Primer/ | or probe or other | |
| probe type | nucleotide (5′-3′) | SEQ ID NO |
| AGES forward | GTYRCTCGGGRAGAGCG | SEQ ID NO: 7 |
| primer | | |
| AGES reverse | CGCCGRCAGTCACCTGT | SEQ ID NO: 8 |
| primer | | |

In some embodiments, each primer of the first collection of primers has a length of no more than 35 nucleotides. In some embodiments, each primer of the second collection of primers has a length of no more than 35 nucleotides. In some embodiments, each primer of the third collection of primers has a length of no more than 35 nucleotides.

In some embodiments, the primer or nucleic acid according to the present disclosure comprises a sequence that is at least about 70% or more, e.g., about 80%, about 900, about 91%, about 92%, about 93%, about 94%, about 95%, about 96%, about 97%, about 98%, or about 99% identical to any of the nucleic acids described herein.

In some embodiments, the determining step further comprises comparing a presence of the PCR product(s) in the at least one test sample with an absence of the PCR product(s) from a negative sample that lacks microorganism nucleic acid, wherein detection of the complex is indicative of the presence of one or more Alicyclobacillus microorganisms. In some embodiments, the method comprises determining a background level of signal generated by the label in the negative sample that lacks microorganism nucleic acid and comparing the background level of signal with the level of signal detected in the test sample. A level of signal that is higher or lower in the test sample as compared to that measured in the negative sample may be indicative of the presence of one or more microorganisms.

In an embodiment, the method optionally comprises comparing an amount of the PCR product(s) having a sequence in the at least one test sample with an amount of a gene having a similar or identical sequence to the sequence of the PCR product(s) from a negative sample that lacks microorganism nucleic acid, wherein an increased amount of gene sequence from the at least one test sample is indicative of the presence of one or more microorganisms. In this regard, the sample is negative for the foodstuff-spoiling microorganism if the amount of the PCR product(s) detected in the sample is no more than the amount of gene sequence that is detected in a negative sample that is known to lack the microorganism nucleic acid. The sample is positive for the foodstuff-spoiling microorganism if the amount of the PCR product(s) detected in the sample is more than the amount of gene sequence that is detected in a negative sample that is known to lack the microorganism nucleic acid.

In some embodiments, the spoilage microorganism detected by the method comprises at least one of: Alicyclobacillus acidocaldarius, Alicyclobacillus contaminans, Alicyclobacillus disulfidooxidans, Alicyclobacillus fastidiosus, Alicyclobacillus ferrooxydans, Alicyclobacillus hesperidum, Alicyclobacillus pomorum. Alicyclobacillus sacchari, Alicyclobacillus sendaiensis, Alicyclobacillus shizuokensis, Alicyclobacillus tolerans, Alicyclobacillus vulcanalis, Alicyclobacillus tengchongenesis, Alicyclobacillus acidoterrestris, Alicyclobacillus acidiphilus, Alicyclobacillus cycloheptanicus, and Alicyclobacillus herbarius.

In some embodiments, the multiplex PCR reaction takes about 5 days or less, 4 days or less, 3 days or less, 2 days or less, or 1 day or less.

The method may comprise detecting microorganisms in any foodstuff. The foodstuff may be, for example, any one or more of dairy products; fats, oils, and fat emulsions; edible ices (including, e.g., sherbet and sorbet); fruits and vegetables (including, e.g., mushrooms and fungi, roots and tubers, pulses and legumes, and aloe vera); seaweeds; nuts and seeds; confectioneries; cereals and cereal products; baked goods (e.g., bread); meat and meat products (including, e.g., poultry and game); fish and fish products (including mollusks, crustaceans, and echinoderms); eggs and egg products; sweeteners, including, e.g., honey and sugar (solid or liquid); salts, spices, soups, sauces, salads, protein products; foodstuffs intended for particular nutritional uses; and beverages (e.g., water, fruit juice, drink, beer, and wine). In a preferred embodiment, the foodstuff is a beverage, typically containing a fruit juice.

The method may comprise obtaining a sample of the foodstuff to be tested and culturing microorganisms in the sample in any suitable manner. For example, when the foodstuff is fruit juice, the sample may be any one or more of fruit juice concentrate, pasteurized fruit juice, unpasteurized fruit juice, other fruit juice, or any further additives or ingredients of fruit juice. Further additives and ingredients of fruit juice may include, for example, any one or more of water (e.g., condensation water and/or water added to the final juice product), soluble additives, and sweeteners such as, e.g., sugar (solid or liquid).

The method may comprise culturing the microorganisms in any suitable culturing medium as is known in the art. The culturing medium may be selected depending on the nature of the foodstuff and microorganism to be tested. Exemplary culturing media may include yeast-sucrose-glucose (YSG) media, potato dextrose media, and broth for Alicyclobacillus (BAT) media.

The microorganisms may be cultured at any suitable temperature and for any suitable duration as is known in the art. The culturing temperature and duration may be selected depending on the nature of the foodstuff and microorganism to be tested. For example, the microorganisms may be cultured at a temperature of about 40° C. to about 60° C., preferably from about 45° C. to about 50° C. The microorganisms may be cultured for about 1 day to about 14 days, or from about 1 day to about 7 days, or from about 1 day to about 3 days, or from about 1 day to about 2 days.

The method may comprise extracting nucleic acid from the microorganisms in any suitable manner as is known in the art. The nucleic acid may be RNA and/or DNA. The protocol for extracting nucleic acid may be selected depending on the nature of the foodstuff, microorganism, and nucleic acid to be tested as is known in the art. Preferably, the nucleic acid is extracted in any manner that lyses Gram positive and Gram negative bacteria and which recovers a testable amount of DNA without using polymerase chain reaction (PCR) inhibitors. The nucleic acid extraction may be carried out using any of a variety of commercially available nucleic acid extraction kits according to the manufacturer's instructions.

In some embodiments, a method for detecting a spoilage microorganism in a sample of foodstuff, the method comprising: (a) contacting a set of oligonucleotides with the sample, wherein the set of oligonucleotides comprising: a first collection of primers targeted for nucleic acids of a guaiacol-producing microorganism; a second collection of primers targeted for nucleic acids of Alicyclobacillus acidoterrestris; and a third primer targeted for nucleic acids of the 16 S rRNA gene common to the genus Alicyclobacillus; (b) amplifying DNA in the sample with the said set of oligonucleotides under a multiplex polymerase chain reaction (PCR); and (c) determining the presence of PCR products of step (b), wherein the presence of a PCR product in the sample is indicative of contamination of the sample by a guaiacol-producing microorganism, the specific species Alicyclobacillus acidoterrestris, and the genus Alicyclobacillus.

In some aspects, the present disclosure provides a system or an assay kit comprising one or more collections of nucleic acids for the specific detection of a spoilage microorganism in a sample of foodstuff. The assay kit may comprise one or more collections of nucleic acids for the detection of any one or more of the guaiacol-producing microorganism, in combination with any one or more Alicyclobacillus species described herein, and/or in combination with any of the genus-specific collections described herein. In some embodiments, the system or assay kit may further comprise any one or more of DNA polymerase, buffer, deoxyribonucleotide triphosphates (dNTP), Mg, etc. The DNA polymerase is not limited and may be any DNA polymerase suitable for qPCR or PCR. Exemplary DNA polymerases include Thermus aquaticus (Taq) polymerase and Pyrococcus furiosus (Pfu) polymerase. The buffer is not limited and may be any buffer suitable for PCR or qPCR. Exemplary buffers include a combination of KCl, Tris and MgCl₂ and a combination of (NH₄)₂SO₄, Tris-HCl, MgCl₂, β-Mercaptoethanol, and EDTA.

In some embodiments, the present disclosure provides a support comprising any of the nucleic acids or collections of nucleic acids described herein immobilized on the support. In some embodiments, a support comprises the sample to be tested immobilized on the support, and the one or more nucleic acid or one or more collection of nucleic acids described herein is applied to the support. The support may be any support suitable for immobilizing the present nucleic acids. The support may further comprise a detectable label. The label may be any label suitable for detecting a complex of the present nucleic acid with microorganism nucleic acid. Exemplary detectable labels may include any one or more of radioactive labels, non-radioactive labels, fluorescent labels, and chemiluminescent labels.

In some embodiments, a system for detecting a spoilage microorganism in a sample of foodstuff, the system comprising a set of oligonucleotides for multiplex polymerase chain reaction (PCR) a comprising: a first collection of primers targeted for nucleic acids of a guaiacol-producing microorganism; and a second collection of primers targeted for nucleic acids of Alicyclobacillus acidoterrestris. In some embodiments, the set of oligonucleotides further comprises: a third collection of primers targeted for nucleic acids of the 16 S rRNA gene common to the genus Alicyclobacillus.

In some embodiments, a kit for detecting a spoilage microorganism in a sample of foodstuff, the kit comprising: a first collection of primers targeted for nucleic acids of a guaiacol-producing microorganism; and a second collection of primers targeted for nucleic acids of Alicyclobacillus acidoterrestris.

In some embodiments, the kit further comprises: an instruction providing a method, the method comprising: (a) contacting the set of oligonucleotides with the sample; (b) amplifying DNA in the sample with the said set of oligonucleotides under a multiplex polymerase chain reaction (PCR); and (c) determining the presence of PCR products of step (b), wherein the presence of a PCR product in the sample is indicative of contamination of the sample by a guaiacol-producing microorganism and the specific species Alicyclobacillus acidoterrestris.

In some embodiments, the kit further comprises: a third collection of primers targeted for nucleic acids of the 16 S rRNA gene common to the genus Alicyclobacillus. In some embodiments, the instruction further provides that: the presence of a PCR product in the sample is indicative of contamination of the sample by a guaiacol-producing microorganism, the specific species Alicyclobacillus acidoterrestris, and the genus Alicyclobacillus.

The term “about” is used in conjunction with numeric values to include normal variations in measurements as expected by persons skilled in the art, and is understood have the same meaning as “approximately” and to cover a typical margin of error, such as ±10% of the stated value. The term “about” also encompasses amounts that differ due to different equilibrium conditions for a composition resulting from a particular initial composition. Whether or not modified by the term “about,” the claims include equivalents to the quantities.

The term “isolated” as used herein means having been removed from its natural environment. The term “purified” as used herein means having been increased in purity, wherein “purity” is a relative term, and not to be necessarily construed as absolute purity. For example, the purity can be at least about 50%, can be greater than 60%, 70% or 80%, 90% or can be 100%.

It should be noted that, as used in this specification and the appended claims, the singular forms “a,” “an,” and “the” include plural referents unless the content clearly dictates otherwise. Thus, for example, reference to a composition containing “a compound” includes having two or more compounds that are either the same or different from each other. It should also be noted that the term “or” is generally employed in its sense including “and/or” unless the content clearly dictates otherwise.

In the interest of brevity and conciseness, any ranges of values set forth in this specification contemplate all values within the range and are to be construed as support for claims reciting any sub-ranges having endpoints which are real number values within the specified range in question. By way of a hypothetical illustrative example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5.

The term “substantially free” may refer to any component that the composition of the disclosure lacks or mostly lacks. When referring to “substantially free” it is intended that the component is not intentionally added to compositions of the disclosure. Use of the term “substantially free” of a component allows for trace amounts of that component to be included in compositions of the disclosure because they are present in another component. However, it is recognized that only trace or de minimus amounts of a component will be allowed when the composition is said to be “substantially free” of that component. Moreover, the term if a composition is said to be “substantially free” of a component, if the component is present in trace or de minimus amounts it is understood that it will not affect the effectiveness of the composition. It is understood that if an ingredient is not expressly included herein or its possible inclusion is not stated herein, the disclosure composition may be substantially free of that ingredient. Likewise, the express inclusion of an ingredient allows for its express exclusion thereby allowing a composition to be substantially free of that expressly stated ingredient.

The methods, compositions, and kits of the present disclosure may comprise, consist essentially of, or consist of the constituents of the present disclosure as well as other constituents described herein. As used herein, “consisting essentially of” means that the methods and compositions may include additional steps, components or ingredients, but only if the additional steps, components or ingredients do not materially alter the basic and novel characteristics of the claimed processes and compositions.

### EXAMPLES

Certain embodiments of the present disclosure are further described with reference to the following experiments and examples. These experiments, examples, and samples are intended to be merely illustrative of the disclosure and are not intended to limit or restrict the scope of the present disclosure in any way and should not be construed as providing conditions, parameters, reagents, or starting materials that must be utilized exclusively in order to practice the art of the present disclosure.

### Example 1—Multiplex PCR Assay

A multiplex PCR assay was developed for detection of guaiacol production and species of Alicyclobacillus sp. A bank of strains was taken out of ASL culture Collection. In total, 65 strains belonging to ten Alicyclobacillus species were analyzed. All strains were grown on BAT plates at 45° C. for 5 days in aerobic conditions. All strains were tested for production of guaiacol by using Doehler guaiacol kit. All strains were tested to confirm the designated species by MALDI-TOF analysis or 16 S PCR sequencing. To isolate DNA, Sigma Aldrich Genomic DNA extraction kit was used, and the presence of DNA was confirmed by gel electrophoresis.

To further develop the PCR test for detection of the guaiacol producing genes, a literature search provided with primers to detect the presence of gene encoding vanillin decarboxylase (vdc), enzyme involved in guaiacol anabolism (Dekowska et al., 2018). Although variations of the primers and amplicon lengths were tested, the best results were obtained with Bur5/6 primers.

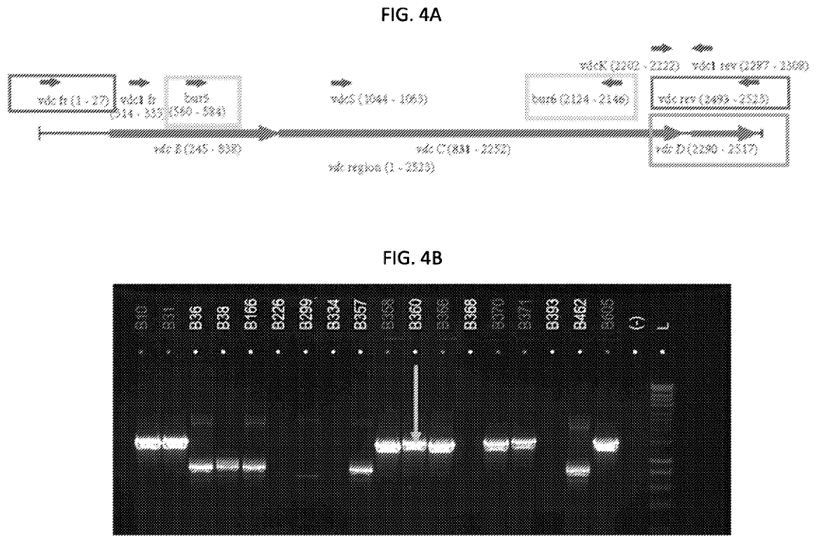

All strains that produced guaiacol produced positive PCR result with the amplicon of 1500 bp, except strain A. acidiphilus B360, which was found to have negative phenotype but positive genotype for guaiacol production. This has been observed in other studies, where a strain of known non-guaiacol producer A. fastidiosus DSM17978 shows band corresponding to the vdc gene (Wang et al., 2021). In the present study, other strains of this species showed results that were in line with phenotypic assay results, as shown in FIGS. 4A-4B.

To develop a pair of primers which provide a band specific to Alicyclobacillus acidoterrestris, the most common and significant producer of guaiacol, alignments of genes such as rpoB and gyrB were performed. Sequencies of relevant species were downloaded from NCBI database, and ClustalW was used as an algorithm for alignment.

Initially, primers for rpoB gene encoding beta subunit of bacterial RNA Polymerase were designed. However, a single band was detected in only 75% of A. acidoterrestris strains. In response, primers were designed based on the alignment of gyrR gene encoding beta subunit of DNA gyrase. All A. acidoterrestris strains produced positive reaction, while all non-acidoterrestris strains produced negative reaction.

In the next step, multiplexing of the two reactions (for guaiacol production and specific for A. acidoterrestris) were combined. The conditions were optimized and the results showed success in performing the two reactions in a single tube, as shown in FIG. 5.

In continuation, sequences of 16 S rRNA genes were downloaded, to include all Alicyclobacillus species tested and representatives of closely related genera (Bacillus, Desulfuribacillus, Geobacillus). Degenerative primers were designed based on the alignment performed as described above. Primers were named AGES F (SEQ ID NO. 7) and AGES R (SEQ ID NO. 8), respectively.

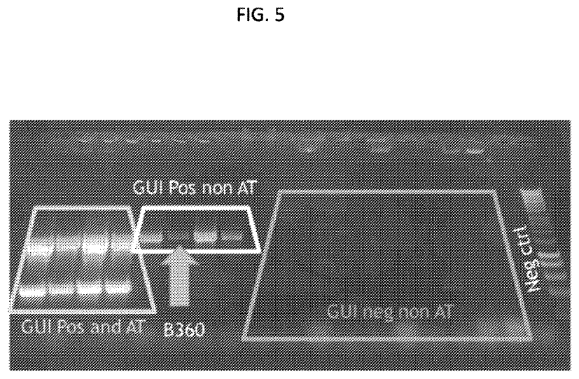

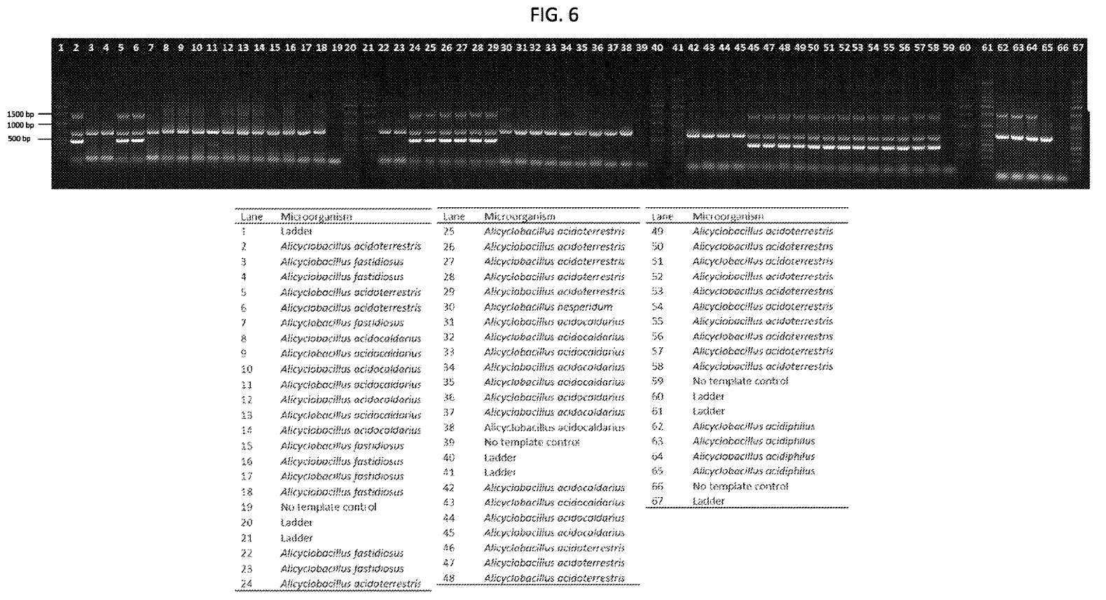

Additional testing was performed to multiplex three primer sets [Bur516 (SEQ ID NOS. 1-2), gyrB (SEQ ID NOS. 5-6), and AGES FIR (SEQ ID NOS. 7-8)] to determine if all three targets would be detected. A band was observed in lanes that indicated that Alicyclobacillus isolates were tested, showing that the AGES primer set successfully identified members of the Alicyclobacillus genus. The isolates identified in the tables as A. acidoterrestris were also determined to be guaiacol-positive through culture-based biochemical testing. The isolates identified as A. acidoterrestris exhibited three bands when the gel was imaged: One to show it belongs to the genus Alicyclobacillus, one to indicate that the isolate has the gene responsible for guaiacol production, and a band indicating that the organism was a member of the species A. acidoterrestris, as shown in FIG. 6.

The above results support that the primer sets showed no amplification of bands that were not specific to the isolates tested, and that multiplexing the tested primers led to results that were both inclusive and exclusive for the targeted attributes.

### NON-PATENT REFERENCES

CONNOR, C. J., LUO, H., GARDENER, B. B. & WANG, H. H. 2005. Development of a real-time PCR-based system targeting the 16S rRNA gene sequence for rapid detection of Alicyclobacillus spp. in juice products. Int J Food Microbiol, 99, 229-35.
DEKOWSKA, A., NIEZGODA, J. & SOKOLOWSKA, B. 2018. Genetic Heterogeneity of Alicyclobacillus Strains Revealed by RFLP Analysis of vdc Region and rpoB Gene. Biomed Res Int, 2018, 9608756.
LI, H., CHEN, H., LIU, B., CAI, R., JIANG, N., YUE, T. & WANG, Z. 2021. Establishment of quantitative PCR assays for the rapid detection of Alicyclobacillus spp. that can produce guaiacol in apple juice. Int J Food Microbiol, 109329.
LUO, IT, YOUSEF, A. E. & WANG, H. 1.1.2004. A real-time polymerase chain reaction-based method for rapid and specific detection of spoilage Alicyclobacillus spp. in apple juice. Lett Appl Microbiol, 39, 376-82.
WANG, Z., YUE, T., YUAN, Y, ZHANG, Y., GAO, Z. & CAL, R. 2021. Targeting the vanillic acid decarboxylase gene for Alicyclobacillus acidoterrestris quantification and guaiacol assessment in apple juices using real time PCR. International Journal of Food Microbiology. 338, 109006.

All publications, patents and patent applications mentioned in the specification are indicative of the level of those skilled in the art to which this disclosure pertains.

The following numbered clauses define further example aspects and features of the present disclosure:

1. A method for detecting a spoilage microorganism in a sample of foodstuff, the method comprising:
(a) contacting a set of oligonucleotides with the sample, wherein the set of oligonucleotides comprising:
   a first collection of primers targeted for nucleic acids of a guaiacol-producing microorganism; and
   second collection of primers targeted for nucleic acids of Alicyclobacillus acidoterrestris;
(b) amplifying DNA in the sample with the said set of oligonucleotides under a multiplex polymerase chain reaction (PCR); and
(c) determining the presence of PCR products of step (b), wherein the presence of a PCR product in the sample is indicative of contamination of the sample by both guaiacol-producing microorganism and Alicyclobacillus acidoterrestris.
   2. The method of clause 1, wherein the set of oligonucleotides further comprising a third collection of primers targeted for nucleic acids of the 16 S rRNA gene common to the genus Alicyclobacillus, and wherein the presence of the PCR product in the sample is indicative of contamination of the sample by a guaiacol-producing microorganism, the specific species Alicyclobacillus acidoterrestris, and the genus Alicyclobacillus.
   3. The method of any one of clauses 1-2, wherein the guaiacol-producing gene encodes vanillin decarboxylase (vdc).
   4 The method of clause 3, wherein the guaiacol-producing gene encoding vanillin decarboxylase (vdc) has at least 75% identity to the nucleic acid sequence set forth in SEQ ID NO. 51.
   5. The method of any one of clauses 1-4, wherein the second collection of primers is targeted for the rpoB gene and/or the gyrB gene of Alicyclobacillus acidoterrestris.
   6. The method of clause 5, wherein the rpoB gene comprises a nucleic acid sequence that has at least 75% identity to the nucleic acid sequence set forth in SEQ ID NOS 9-14.
   7. The method of any one of clauses 5-6, wherein the gyrB gene comprises a nucleic acid sequence that has at least 75% identity to the nucleic acid sequence set forth in SEQ ID NOS. 15-26.
   8. The method of any one of clauses 2-7, wherein the 16 S rRNA gene comprises a nucleic acid sequence that has at least 75% identity to the nucleic acid sequence set forth in SEQ ID NOS. 27-50.
   9 The method of any one of clauses 1-8, wherein the first collection of primers comprises at least one primer having at least 75% identity to the nucleic acid sequence set forth in SEQ ID NOS. 1-2.
   10. The method of any one of clauses 1-9, wherein the second collection of primers comprises at least one primer having at least 75% identity to the nucleic acid sequence set forth in SEQ ID NOS. 3-6.
   11. The method of any one of clauses 1-10, wherein the third collection of primers comprises at least one primer having at least 75% identity to the nucleic acid sequence set forth in SEQ ID NOS. 7-8.
   12. The method of any one of clauses 1-11, wherein the first collection of primers hybridizes with the nucleic acids of the guaiacol-producing gene.
   13. The method of any one of clauses 1-12, wherein the second collection of primers hybridizes with the nucleic acids of the Alicyclobacillus acidoterrestris.
   14. The method of any one of clauses 1-13, wherein the third collection of primers hybridizes with the nucleic acids of the 16 S rRNA gene.
   15. The method of any one of clauses 1-14, wherein the second collection of primers comprises at least one of: a forward primer having at least 75% identity to the nucleic acid sequence set forth in SEQ ID NOS. 3 and 5.
   16. The method of any one of clauses 1-15, wherein the second collection of primers comprises at least one of: a reverse primer having at least 75% identity to the nucleic acid sequence set forth in SEQ ID NOS. 4 and 6.
   17. The method of any one of clauses 2-16, wherein the third collection of primers comprises at least one of: a forward primer having at least 75% identity to the nucleic acid sequence set forth in SEQ ID NO. 7.
   18 The method of any one of clauses 2-17, wherein the third collection of primers comprises at least one of: a reverse primer having at least 75% identity to the nucleic acid sequence set forth in SEQ ID NO. 8.
   19. The method of any one of clauses 1-18, wherein each primer of the first collection of primers has a length of no more than 35 nucleotides.
   20. The method of any one of clauses 1-19, wherein each primer of the second collection of primers has a length of no more than 35 nucleotides.
   21. The method of any one of clauses 2-20, wherein each primer of the third collection of primers has a length of no more than 35 nucleotides.
   22. The method of any one of clauses 1-21, wherein the spoilage microorganism detected by the method comprise at least one of Alicyclobacillus acidocaldarius, Alicyclobacillus contaminans, Alicyclobacillus disulfidooxidans, Alicyclobacillus fastidiosus, Alicyclobacillus ferrooxydans, Alicyclobacillus hesperidem, Alicyclobacillus pomorum, Alicyclobacillus sacchari, Alicyclobacillus sendaiensis, Alicyclobacillus shizuokensis, Alicyclobacillus tolerans, Alicyclobacillus vulcanalis, Alicyclobacillus tengchongenesis, Alicyclobacillus acidoterrestris, Alicyclobacillus acidiphilus, Alicyclobacillus cycloheptanicus, and Alicyclobacillus herbarius.
   23. The method of any one of clauses 1-22, wherein the multiplex PCR reaction takes about 5 days or less, 4 days or less, 3 days or less, 2 days or less, or 1 day or less.
   24. The method of any one of clauses 1-23, wherein the foodstuff is a beverage.
   25. The method of any one of clauses 1-24, wherein the foodstuff is fruit juice.
   26. The method of any one of clauses 1-25, wherein the PCR is quantitative PCR.
   27. The method of any one of clauses 1-26, wherein the PCR is real-time PCR.
   28. A method for detecting a spoilage microorganism in a sample of foodstuff, the method comprising:
(a) contacting a set of oligonucleotides with the sample, wherein the set of oligonucleotides comprising:
   a first collection of primers targeted for nucleic acids of a guaiacol-producing microorganism;
   a second collection of primers targeted for nucleic acids of Alicyclobacillus acidoterrestris; and
   a third primer targeted for nucleic acids of the 16 S rRNA gene common to the genus Alicyclobacillus,
(b) amplifying DNA in the sample with the said set of oligonucleotides under a multiplex polymerase chain reaction (PCR);
(c) determining the presence of PCR products of step (b), wherein the presence of a PCR product in the sample is indicative of contamination of the sample by a guaiacol-producing microorganism, the specific species Alicyclobacillus acidoterrestris, and the genus Alicyclobacillus.
   29. The method of clause 28, wherein the first collection of primers comprises at least one primer having at least 75% identity to the nucleic acid sequence set forth in SEQ ID NOS 1-2.
   30. The method of any one of clauses 28-29, wherein the second collection of primers comprises at least one primer having at least 75% identity to the nucleic acid sequence set forth in SEQ ID NOS. 3-6.
   31. The method of any one of clauses 28-30, wherein the third collection of primers comprises at least one primer having at least 75% identity to the nucleic acid sequence set forth in SEQ ID NOS. 7-8.
   32. The method of any one of clauses 28-31, wherein each primer of the first collection of primers has a length of no more than 35 nucleotides.
   33. The method of any one of clauses 28-32, wherein each primer of the second collection of primers has a length of no more than 35 nucleotides.
   34. The method of any one of clauses 28-33, wherein each primer of the third collection of primers has a length of no more than 35 nucleotides.
   35. A system for detecting a spoilage microorganism in a sample of foodstuff, the system comprising a set of oligonucleotides for multiplex polymerase chain reaction (PCR), wherein the set of oligonucleotides comprises,
a first collection of primers targeted for nucleic acids of a guaiacol-producing microorganism; and
a second collection of primers targeted for nucleic acids of Alicyclobacillus acidoterrestris.
   36. The system of clause 35, wherein the set of oligonucleotides further comprises: a third collection of primers targeted for nucleic acids of the 16 S rRNA gene common to the genus Alicyclobacillus.
   37. The system of any one of clauses 35-36, wherein the first collection of primers comprises at least one primer having at least 75% identity to the nucleic acid sequence set forth in SEQ ID NOS. 1-2.
   38. The system of any one of clauses 35-37, wherein the second collection of primers comprises at least one primer having at least 75% identity to the nucleic acid sequence set forth in SEQ ID NOS. 3-6.
   39. The system of any one of clauses 36-38, wherein the third collection of primers comprises at least one primer having at least 75% identity to the nucleic acid sequence set forth in SEQ ID NOS. 7-8.
   40. A kit for detecting a spoilage microorganism in a sample of foodstuff, the kit comprising.
a first collection of primers targeted for nucleic acids of a guaiacol-producing microorganism; and
a second collection of primers targeted for nucleic acids of Alicyclobacillus acidoterrestris.
   41. The kit of clause 40, further comprising: an instruction providing a method, the method comprising:
(a) contacting the set of oligonucleotides with the sample;
(b) amplifying DNA in the sample with the said set of oligonuclotides under a multiplex polymerase chain reaction (PCR);
(c) determining the presence of PCR products of step (b), wherein the presence of a PCR product in the sample is indicative of contamination of the sample by a guaiacol-producing microorganism and the specific species Alicyclobacillus acidoterrestris.
   42. The kit of any one of clauses 40-41, wherein the first collection of primers comprises at least one primer having at least 75% identity to the nucleic acid sequence set forth in SEQ ID NOS. 1-2.
   43. The kit of any one of clauses 40-42, wherein the second collection of primers comprises at least one primer having at least 75% identity to the nucleic acid sequence set forth in SEQ ID NOS. 3-6.
   44. The kit of any one of clauses 40-43, further comprising: a third collection of primers targeted for nucleic acids of the 16 S rRNA gene common to the genus Alicyclobacillus.
   45. The kit of clause 44, wherein the third collection of primers comprises at least one primer having at least 75% identity to the nucleic acid sequence set forth in SEQ ID NOS. 7-8.
   46. The kit of any one of clauses 44-45, wherein the instruction further provides that: the presence of a PCR product in the sample is indicative of contamination of the sample by a guaiacol-producing microorganism, the specific species Alicyclobacillus acidoterrestris, and the genus Alicyclobacillus.

Although only exemplary embodiments of the present disclosure have been described in detail above, those skilled in the art will readily appreciate that many modifications are possible without materially departing from the novel teachings and advantages of this invention. Accordingly, all such modifications are intended to be included within the scope of this disclosure as defined in the following claims.

What is claimed is:

1. A method for detecting a spoilage microorganism in a sample of foodstuff, the method comprising:
(a) contacting a set of oligonucleotides with the sample, wherein the set of oligonucleotides comprising:
a first collection of primers targeted for nucleic acids of a guaiacol-producing microorganism; and
a second collection of primers targeted for nucleic acids of Alicyclobacillus acidoterrestris;
(b) amplifying DNA in the sample with the said set of oligonucleotides under a multiplex polymerase chain reaction (PCR); and
(c) determining the presence of PCR products of stop (b), wherein the presence of a PCR product in the sample is indicative of contamination of the sample by both guaiacol-producing microorganism and Alicyclobacillus acidoterrestris.

2. The method of claim 1, wherein the set of oligonucleotides further comprising a third collection of primers targeted for nucleic acids of the 16 S rRNA gene common to the genus Alicyclobacillus, and wherein the presence of the PCR product in the sample is indicative of contamination of the sample by a guaiacol-producing microorganism, the specific species Alicyclobacillus acidoterrestris, and the genus Alicyclobacillus.

3. The method of any one of claims 1-2, wherein the guaiacol-producing gene encodes vanillin decarboxylase (vdc).

4. The method of claim 3, wherein the guaiacol-producing gene encoding vanillin decarboxylase (vdc) has at least 75% identity to the nucleic acid sequence set forth in SEQ ID NO. 51.

5. The method of any one of claims 1-4, wherein the second collection of primers is targeted for the rpoB gene and/or the gyrB gene of Alicyclobacillus acidoterrestris.

6. The method of claim 5, wherein the rpoB gene comprises a nucleic acid sequence that has at least 75% identity to the nucleic acid sequence set forth in SEQ ID NOS 9-14.

7. The method of any one of claims 5-6, wherein the gyrB gene comprises a nucleic acid sequence that has at least 75% identity to the nucleic acid sequence set forth in SEQ ID NOS. 15-26.

8. The method of any one of claims 2-7, wherein the 16 S rRNA gene comprises a nucleic acid sequence that has at least 75% identity to the nucleic acid sequence set forth in SEQ ID NOS. 27-50.

9. The method of any one of claims 1-8, wherein the first collection of primers comprises at least one primer having at least 75% identity to the nucleic acid sequence set forth in SEQ ID NOS 1-2.

10. The method of any one of claims 1-9, wherein the second collection of primers comprises at least one primer having at least 75% identity to the nucleic acid sequence set forth in SEQ ID NOS. 3-6.

11. The method of any one of claims 1-10, wherein the third collection of primers comprises at least one primer having at least 75% identity to the nucleic acid sequence set forth in SEQ ID NOS. 7-8.

12. The method of any one of claims 1-11, wherein the first collection of primers hybridizes with the nucleic acids of the guaiacol-producing gene.

13. The method of any one of claims 1-12, wherein the second collection of primers hybridizes with the nucleic acids of the Alicyclobacillus acidoterrestris.

14. The method of any one of claims 1-13, wherein the third collection of primers hybridizes with the nucleic acids of the 16 S rRNA gene.

15. The method of any one of claims 1-14, wherein the second collection of primers comprises at least one of: a forward primer having at least 75% identity to the nucleic acid sequence set forth in SEQ ID NOS. 3 and 5.

16. The method of any one of claims 1-15, wherein the second collection of primers comprises at least one of: a reverse primer having at least 75% identity to the nucleic acid sequence set forth in SEQ ID NOS. 4 and 6.

17. The method of any one of claims 2-16, wherein the third collection of primers comprises at least one of: a forward primer having at least 75% identity to the nucleic acid sequence set forth in SEQ ID NO. 7.

18. The method of any one of claims 2-17, wherein the third collection of primers comprises at least one of: a reverse primer having at least 75% identity to the nucleic acid sequence set forth in SEQ ID NO. 8.

19. The method of any one of claims 1-18, wherein each primer of the first collection of primers has a length of no more than 35 nucleotides.

20. The method of any one of claims 1-19, wherein each primer of the second collection of primers has a length of no more than 35 nucleotides.

21. The method of any one of claims 2-20, wherein each primer of the third collection of primers has a length of no more than 35 nucleotides.

22. The method of any one of claims 1-21, wherein the spoilage microorganism detected by the method comprise at least one of Alicyclobacillus acidocaldarius, Alicyclobacillus contaminans, Alicyclobacillus disulfidooxidans, Alicyclobacillus fastidiosus, Alicyclobacillus ferrooxydans, Alicyclobacillus hesperidem, Alicyclobacillus pomorum, Alicyclobacillus sacchari, Alicyclobacillus sendaiensis, Alicyclobacillus shizuokensis, Alicyclobacillus tolerans, Alicyclobacillus vulcanalis, Alicyclobacillus tengchongenesis, Alicyclobacillus acidoterrestris, Alicyclobacillus acidiphilus, Alicyclobacillus cycloheptanicus, and Alicyclobacillus herbarius.

23. The method of any one of claims 1-22, wherein the multiplex PCR reaction takes about 5 days or less, 4 days or less, 3 days or less, 2 days or less, or 1 day or less.

24. The method of any one of claims 1-23, wherein the foodstuff is a beverage.

25. The method of any one of claims 1-24, wherein the foodstuff is fruit juice.

26. The method of any one of claims 1-25, wherein the PCR is quantitative PCR.

27. The method of any one of claims 1-26, wherein the PCR is real-time PCR.

28. A method for detecting a spoilage microorganism in a sample of foodstuff, the method comprising:
(a) contacting a set of oligonucleotides with the sample, wherein the set of oligonucleotides comprising:
a first collection of primers targeted for nucleic acids of a guaiacol-producing microorganism;
a second collection of primers targeted for nucleic acids of Alicyclobacillus acidoterrestris; and
a third primer targeted for nucleic acids of the 16 S rRNA gene common to the genus Alicyclobacillus;
(b) amplifying DNA in the sample with the said set of oligonucleotides under a multiplex polymerase chain reaction (PCR);
(c) determining the presence of PCR products of step (b), wherein the presence of a PCR product in the sample is indicative of contamination of the sample by a guaiacol-producing microorganism, the specific species Alicyclobacillus acidoterrestris, and the genus Alicyclobacillus.

29. The method of claim 28, wherein the first collection of primers comprises at least one primer having at least 75% identity to the nucleic acid sequence set forth in SEQ ID NOS. 1-2.

30. The method of any one of claims 28-29, wherein the second collection of primers comprises at least one primer having at least 75% identity to the nucleic acid sequence set forth in SEQ ID NOS. 3-6.

31. The method of any one of claims 28-30, wherein the third collection of primers comprises at least one primer having at least 75% identity to the nucleic acid sequence set forth in SEQ ID NOS. 7-8.

32. The method of any one of claims 28-31, wherein each primer of the first collection of primers has a length of no more than 35 nucleotides.

33. The method of any one of claims 29-32, wherein each primer of the second collection of primers has a length of no more than 35 nucleotides.

34. The method of any one of claims 28-33, wherein each primer of the third collection of primers has a length of no more than 35 nucleotides.

35. A system for detecting a spoilage microorganism in a sample of foodstuff, the system comprising a set of oligonucleotides for multiplex polymerase chain reaction (PCR), wherein the set of oligonucleotides comprises:
a first collection of primers targeted for nucleic acids of a guaiacol-producing microorganism; and
a second collection of primers targeted for nucleic acids of Alicyclobacillus acidoterrestris.

36. The system of claim 35, wherein the set of oligonucleotides further comprises: a third collection of primers targeted for nucleic acids of the 16 S rRNA gene common to the genus Alicyclobacillus.

37. The system of any one of claims 35-36, wherein the first collection of primers comprises at least one primer having at least 75% identity to the nucleic acid sequence set forth in SEQ ID NOS. 1-2.

38. The system of any one of claims 35-37, wherein the second collection of primers comprises at least one primer having at least 75% identity to the nucleic acid sequence set forth in SEQ ID NOS. 3-6.

39. The system of any one of claims 36-38, wherein the third collection of primers comprises at least one primer having at least 75% identity to the nucleic acid sequence set forth in SEQ ID NOS. 7-8.

40. A kit for detecting a spoilage microorganism in a sample of foodstuff, the kit comprising
a first collection of primers targeted for nucleic acids of a guaiacol-producing microorganism; and
a second collection of primers targeted for nucleic acids of Alicyclobacillus acidoterrestris.

41. The kit of claim 40, further comprising: an instruction providing a method, the method comprising:
(a) contacting the set of oligonucleotides with the sample,
(b) amplifying DNA in the sample with the said set of oligonucleotides under a multiplex polymerase chain reaction (PCR);
(c) determining the presence of PCR products of step (b), wherein the presence of a PCR product in the sample is indicative of contamination of the sample by a guaiacol-producing microorganism and the specific species Alicyclobacillus acidoterrestris.

42. The kit of any one of claims 40-41, wherein the first collection of primers comprises at least one primer having at least 75% identity to the nucleic acid sequence set forth in SEQ ID NOS. 1-2.

43. The kit of any one of claims 40-42, wherein the second collection of primers comprises at least one primer having at least 75% identity to the nucleic acid sequence set forth in SEQ ID NOS. 3-6.

44. The kit of any one of claims 40-43, further comprising: a third collection of primers targeted for nucleic acids of the 16 S rRNA gene common to the genus Alicyclobacillus.

45. The kit of claim 44, wherein the third collection of primers comprises at least one primer having at least 75% identity to the nucleic acid sequence set forth in SEQ ID NOS. 7-8.

46. The kit of any one of claims 44-45, wherein the instruction further provides that: the presence of a PCR product in the sample is indicative of contamination of the sample by a guaiacol-producing microorganism, the specific species Alicyclobacillus acidoterrestris, and the genus Alicyclobacillus.


SEQUENCE LISTING (51 sequences)

SEQ ID NO: 1 - DNA, 24 bp, synthetic construct
  misc_feature 1..24: note = synthetic construct
  source 1..24: mol_type = other DNA
ccgacgtgatgctcaargagcgca

SEQ ID NO: 2 - DNA, 23 bp, synthetic construct
  misc_feature 1..23: note = synthetic construct
  source 1..23: mol_type = other DNA
gtsgcrtcgagaatcatcttgtg

SEQ ID NO: 3 - DNA, 28 bp, synthetic construct
  misc_feature 1..28: note = synthetic construct
  source 1..28: mol_type = other DNA
ccgtcgtgaggacgttatcgcggtgccg

SEQ ID NO: 4 - DNA, 25 bp, synthetic construct
  misc_feature 1..25: note = synthetic construct
  source 1..25: mol_type = other DNA
cgcaacgtcaccgcgaacaagtcgg

SEQ ID NO: 5 - DNA, 25 bp, synthetic construct
  misc_feature 1..25: note = synthetic construct
  source 1..25: mol_type = other DNA
gttcccaagtacgatttgaaagtgg

SEQ ID NO: 6 - DNA, 23 bp, synthetic construct
  misc_feature 1..23: note = synthetic construct
  source 1..23: mol_type = other DNA
ggttgctctcgctccccttaatc

SEQ ID NO: 7 - DNA, 17 bp, synthetic construct
  misc_feature 1..17: note = synthetic construct
  source 1..17: mol_type = other DNA
gtyrctcgggragagcg

SEQ ID NO: 8 - DNA, 17 bp, synthetic construct
  misc_feature 1..17: note = synthetic construct
  source 1..17: mol_type = other DNA
cgccgrcagtcacctgt

SEQ ID NO: 9 - DNA, 769 bp, Alicyclobacillus acidocaldarius
  source 1..769: mol_type = other DNA
acatcggcctcatcaactcgctctcgacctttgcgcgcgtcaacgagtacggcttcatcgagacgccataccggcgcgtcgatccagagacgggcgtggtcacggatcagattgattacctgacggcggatgaggaggaaaactacctcattgcgcaggcgaacgagcctctgacagaggacggtcatttcgtctccgaggagatcacggtgcgctcgcgcgaggacgtgatcaccgtgagccgagatcgaattgactacatggacgtgtcgccgaagcaggtggtgtccgttgcgacggcgctcatcccgttccttgaaaacgacgacgcgaaccgcgcgctcatgggatcgaacatgcagcgccaggcagtgccgctgctcgtgacggattcgccgttcgtggggacggggatggagtaccaggcggcgaaggactccggggtttgcgtggtttccaagcacaatggcgtcgtggagcgtgtgacggcccgcgagatctgggtgcgcgaagaggccgtcgtggatgggcaggtcgtgaaggggaacatccacaaatacaagctgatcaagttcgcgcgttcgaaccagaacacgtgcctgaaccagaggcccatcgtgcgcgagggcgatcgcgtcaaagttggggacattctcgcggacggcccggcgacacaaaagggcgagctggcgctcggccggaacgtgctcgtcgcgttcatgacctgggagggctacaactacgaggacgcgattctcatctcggaaaa

SEQ ID NO: 10 - DNA, 3531 bp, synthetic construct
  source 1..3531: mol_type = other DNA
ttgcagggacacatggtcaagtacggatgggcggagcgccgcagttacgcgcgcattcgcgaggtcctcgatctcccaaacctcattgagatccagcagaagtcgtacgagtggttcctgcgggaaggactgcgcgagaccttcgcggacatctctcccatcacggacttcaccgggaaccttgtgctcgaattcgtggattacagcctcggcgagcccaagtacgacgtggaagaatcgaaggagcgcgatgtgacttacgcggcgccgcttcgcgtgaaagttcgtctgctcaacaaggagacgggcgaggtcaaagagcaagaggtgtttctcggcgactttccgctcatgaccgagaccggcaccttcatcatcaacggtgcggagcgcgtcatcgtaagccagctcgtccgctcccccagcgtctattatagcagcaagatcgacaagaatggcaaacgtacgttcgcggccacggtgattcccaatcggggcgcatggctcgaatttgagacggacgcgaaggacgtcgtgtacgtccgaattgaccgcacccgcaagttgcccatcacggtcctgctccgggcgctcggcttgtcgtcggacgccgagatcatcgaactgttgggcgaggacgagtacttgcagaatacgctggacaaggataccacggactcgacggagcgcgcgctcatcgagatttatgagcgcttgcgcccgggtgagccgccgacggtggagaacgcgcgtgccctgcttgcgtcgcggttcttcgatcccaagcggtacgatctcgccgcggttgggcggtacaagatcaacaagaagctccatctgaaaaatcgcctgttgaatcagcgcttggcggagaccatcgtcgacgaagagacgggcgagatcattgccgaagccggggccgtcttggaccgccgcacgctggaccggttgattcctcgcctgtcgggcagggttgggcgattcagcatccgcggcacgcgcgacttgtttgagcacgacgagattccgctgcagatggtgaagatcttcagtccggcggaggacggaaagattctgcacgtcatctcgaacggagaactgccgtcggacgtcaaatacatcacgccgtccgacatcattgccgccatctcgtacttcttcaacctgctgcgcggcgttgggacgacggatgacattgatcacctgggcaaccggcgcctccggtcggtgggcgagctgttgcagaatcagttccgcatcgggctctcccgcatggagcgcgtggtgcgcgagcgcatgtccattcaggacgcgagcgccatcacaccgcaggcgctgatcaacatccgcccggtgattgcggccatcaaggagttctttggatcgagccagctgtcgcagttcatggatcagacgaatccgctcgccgaactcacgcacaagcggcgtctttcggcgctcgggcccggtggcctcacgcgtgagcgggccggcttcgaagtgcgcgacgtgcactactcgcactacggccgcatgtgccccatcgagacgcccgaaggtccgaacatcggcctcatcaactcgctctcgacctttgcgcgcgtcaacgagtacggcttcatcgagacgccataccggcgcgtcgatccagagacgggcgtggtcacggatcagattgattacctgacggcggatgaggaggaaaactacctcattgcgcaggcgaacgagcctctgacagaggacggtcatttcgtctccgaggagatcacggtgcgctcgcgcgaggacgtgatcaccgtgagccgagatcgaattgactacatggacgtgtcgccgaagcaggtggtgtccgttgcgacggcgctcatcccgttccttgaaaacgacgacgcgaaccgcgcgctcatgggatcgaacatgcagcgccaggcagtgccgctgctcgtgacggattcgccgttcgtggggacggggatggagtaccaggcggcgaaggactccggggtttgcgtggtttccaagcacaatggcgtcgtggagcgtgtgacggcccgcgagatctgggtgcgcgaagaggccgtcgtggatgggcaggtcgtgaaggggaacatccacaaatacaagctgatcaagttcgcgcgttcgaaccagaacacgtgcctgaaccagaggcccatcgtgcgcgagggcgatcgcgtcaaagttggggacattctcgcggacggcccggcgacacaaaagggcgagctggcgctcggccggaacgtgctcgtcgcgttcatgacctgggagggctacaactacgaggacgcgattctcatctcggaaaagatggtgaaggaggacgtctacacgtccatccacatcgaggagtacgaaattgaggcgcgcgacacgaagctcgggccagaggagatcactcgcgacatcccgaacgtcggcgaagacacgctcaaaaacctcgacgaacgcgggatcatccgtatcggcgcggagatcacgacgaacgacatcctcgtcggcaaggtgacgccgaagggcgtgacggagcttaccgcggaagagcggctgctgcacgcgattttcggcgagaaggctcgcgaagtgcgcgacacgtcgctgcgcgtgccgcatggaggtgccgggatcgtcgtggacgtcaaggtgttcacgcgcgagaacggcgacgagctgcccgctggcgtgaaccagttggtgcgcgtctacgtcgcgcagaagcgcaagatctcggagggcgacaagatggccggtcgccacgggaacaagggtgtcgtggcgcgcatcctgccggaggaggatatgccgttcctcgaggatgggacgccagtcgagatcgtcctgaatccgctcggcgtgccgtcgcgtatgaacatcggtcaggtgctggagacgcacctgggcatggccgcgaaggtgctggggctgaagatggccacgccggtgtttgacggcgccaagccggaggacgtgtttgagacgctgcgcgaggctggcttgcccgaggacggcaagcaggtgctctacgacggccggagcggcgagccgttcgaaaaccgcgtcaccgtgggatacgtgtacatgatgaagctgcatcacctggtggacgacaagatccacgcgcgctccacgggtccgtactcgctcgtcacccaacagccgctcggaggcaaggcgcagttcggaggacagcggtttggcgagatggaggtctgggccctggaggcgtacggcgcggcttacacgctgcaggagctcctcacggtcaagtcggacgacgtggtgggacgcgtcaagacgtacgaggccatcgtcaagggcgagaacgtgccggagccgggtgtgccggagtcgttcaaggtgctcatcaaggagctccagagcctggggatggacgtgaagatcttgagcggcgacgagcgcgagatcgagatgaaggagatggacgacgacgaggacagccccgacaagctcaatctgaacctggagtacaacgaggtcggcgattga

SEQ ID NO: 11 - DNA, 850 bp, Alicyclobacillus acidoterrestris
  source 1..850: mol_type = genomic DNA
catcggcttgattaactcgttgtcgacgtatgcgcgaattaacgagtacggatttattgaaacgccgtatcgccgggtagaccctgagaccggcgttgtgacggataaaattgattacctgaccgccgacgaggaagaaaactacgtcgtcgcgcaggcaaatgagccactgacagaagacggtcgactggcgtcagaggaaatcatcgtccgccgtcgtgaggacgttatcgcggtgccgcgggatcggattgactatatggacgtctctccgaaacaggtcgtgtctgtcgcgactgcgttgattccgttcttggaaaacgacgacgcgaaccgcgctttgatgggtgcgaacatgcaacgccaagctgttccgttgctcgtcaccgattcgccgtatgtcggtacgggaattgagcaccaggcggcgaaggattccggcgtctgtgtggtatctcgccacaatggtatcgtcgaacgcgtcacggccaaagaggtctgggtgcgtcaagagaccgaagtggacggccgacttgttcgcggtgacgttgcgaagtaccgcctgacgaagtttacgcgttcgaaccaaaatacgtgtgtaaaccaacggccgattgtccgcgaggggcaacgcgtcaaagtgggcgacattttggcggacggacctgctacgcagaacggtgagctcgcgcttggtcgcaacgtcttggtcgcgtttatgacgtgggaaggctacaactacgaggacgcgattcttctctcggagaagatggtgaaggaagacgtatatacgtcgattcacatcgaggagtacgaactcgaagcgcgcgacacgaagttggggcctgaa

SEQ ID NO: 12 - DNA, 3531 bp, Alicyclobacillus acidiphilus
  source 1..3531: mol_type = genomic DNA
ttgcagggacatatggtcaagtacggatgggcagagcgtcgcagctattcccggatccgtgaagtcctcgatttacccaatctgattgaaatccaacagaaatcgtacgagtggtttttgcgtgaaggcttgcgcgagatgttcgctgacatttcgccgattcaagactttacgggaaatctcgtcctcgagttcatcgactacagcctcggggaacccaagtacgatgtcgaagagtcgaaagagcgtgatgtgacatacgctgcgcctcttcgggtgaaagttcgtctgttgaataaggagacaggcgaggtcaaggagcaggaagttttccttggagactttccgctcatgaccgacactggcacctttatcatcaacggagcagagcgagtcatcgtcagtcagttggtccgttcacctagtgtttattatagcagcaaaatcgacaagaatggcaaacgtacgttcgctgcgacggtgattccgaatcgcggggcgtggttggagtttgaaaccgacgcgaaggacgtggtgtacgtccggatcgaccgtacgcgcaagcttccgattaccgttttgttgcgggcgttaggattgtcgtccgacgctgagattattcaattgctcggcgaagacgaatacctgcagaatacgcttgataaggacacaaccgattcgacggagcgcgccttggttgaaatttatgaacgtctgcgtccgggtgaaccgccgacggtcgaaaatgctcgcgctttgttggcgtcgcgtttctttgatccgaagcggtacgatctcgcgaacgtgggtcgttacaagatcaataagaagcttcatctgaaaaaccgccttctcaaccttcggttggctgagacgctcgtcgacgccgatacgggcgagattgtggcggaggctggaacggtcatcgaccgccgcgttctggatcggttgattccgcttctgaacggcaacgtaggtcgcttcaccgtgcgggctacgcgcgacttgcttgaggatgaagtggtgccgttgcaaatggtgaaggtgttcagtccgtccgaggacggtcggatcctccacatcatcagcaacggtgaaatttcgaccgatgtgaagtacattacaccgtcggacattctcggagcggtttcgtacttcttcaacctcctgcgtggcgtgggcacgacggacgatatcgatcacttgggcaaccgccgtctgcgttctgtgggcgagctgctgcagaaccagttccgcattgggctttctcgcatggagcgcgtggttcgcgagcggatgtcgattcaggacgcgagcgccatcacgccgcaggccttgatcaacattcgcccggtgattgcggctatcaaagaattctttgggtccagtcagttgtcccagttcatggaccagacgaatccgctcgccgaactgacacacaagcggcgcctgtcggcgttgggtcctggcggtttgacgcgtgaacgggctggatttgaggtccgcgacgtgcactattctcactacgggcgcatgtgtccgattgagacgccggaaggtccgaacatcggtttgatcaactcgctgtcgacctatgcgcggatcaacgagtatggcttcatcgagacgccgtaccgccgggtcgatccggagacgggtgtcgtcaccgacaagattgattatctgaccgcggacgaggaagagaattacgtgattgcgcaggcgaacgagcctctcaccgaagatggtcgtctcgcttcggaagagattacagttcgttcccgggaagacgtcatcaaggtctcccgagatcggatcgactacatggacgtttcgccgaagcaggttgtatcggtcgcgaccgcgctcattccgttcctggagaacgacgatgccaaccgcgcgttgatgggatcgaacatgcagcggcaagcggttccgctgttggtcaccgattcgccttacgtgggcacgggcatggaacatcaggcggcgaaggattctggtgtgtgcgtcgtttcccggcacaacggtatcgtggaacgtgtgacggcccgggaagtttggattcgtcaagaggcggaagtcgatgggcagaccattcgcggcgatttggccaagtacaaattgatcaagttcgctcgttccaaccaaaacacctgtgtcaaccagcgtccgattgttcgtgaaggccagcgggtgcgcgtcggcgatatcctcgctgacgggccggcgacgcagaacggcgagctggcgttgggacgcaatgtgctggtcgcgttcatgacctgggaaggttacaactacgaagacgccattctcctgtcggagaagatggtgaaagaagacgtctatacgtcgattcatatcgaagagtacgagttggaagcgcgcgacacgaaactgggtccggaagaaattacccgcgatattccaaacgtcggcgaggatgcgttgaagaacctcgatgagcggggcatcattcgcatcggtgcggaaattcgcacgggtgacatcttggtcggcaaggtaacgccgaagggcgtaaccgaattgaccgctgaagagcggctgctccacgcgattttcggcgagaaggctcgcgaagttcgcgacacgtcactgagagttccgcacggcggcgcggggatcgtcgtcgatgtgaaagtgtttatgcgggagaatggcgacgaactcccggctggcgtcaaccaattggtgcgtgtgtacatcgctcagaaacgaaagatttctgaaggcgataagatggctggacgccacggaaacaagggtgtcgtggcgcggatcctgccggaagaagacatgccgttcctcgaagacggaacgccggttgaaattgtgttgaatccacttggcgtcccgtcccgtatgaacatcgggcaggtgcttgagacgcaccttggcatggcggcgaaagtgcttgggctgaaaatggctacgccggtcttcgacggtgcccgctcagaggacgtcttcgagacgctcaaagaagcgaacctgccggaagacggcaagcagattctcttcgatggacgcagcggcgaaccgtttgagaaccgcgtgacagttggttacgtctacatgctgaagctgcaccacttggtcgacgacaagatccatgcgcgatccacagggccgtactcgttggtcacgcagcaaccgcttggcggtaaggctcaattcggcgggcagcgcttcggcgagatggaagtgtgggccttggaggcgtacggtgccgcttatacgctgcaggaaattctcacggtcaagtcggacgacgtcgttggacgcgtgaaaacctacgaggcgattgtcaagggcgagaatgtgccggaaccgggtgttccagagtcgtttaaagtcctcatcaaagaacttcagagtctcggcatggatgtgaagatcctgagcggtgacgagcgcgagatcgaaatgaaggaaatcgacgaggaagacgacggatcggagaagctcaacctgaacctcgagtacaacgaagtcggagaataa

SEQ ID NO: 13 - DNA, 3531 bp, Alicyclobacillus sacchari
  source 1..3531: mol_type = genomic DNA
ttgcagggacatatggtcaagtacggatgggcggagcgtcgcagctactcgcgcattcgtgaagtcctcgatctgcccaacctgattgaaatccagcaaaaatcgtatgaatggttcttgcgtgaaggactccgtgaaatgtttgccgacatttcgccgattcaggacttcacgggcaatctggtactcgaattcatcgactacagtctcggcgagccgaaatatgacgtcgaagagtcgaaagaacgcgatgtcacgtatgccgcaccgcttcgtgtcaaagtgcgtttgctgaataaagagaccggggaagtgaaggaacaagaagtgttccttggcgactttccactaatgacagatacgggcacgttcatcatcaacggtgctgaacgcgtcatcgtcagtcagttggttcgttcgcctagtgtctattatagcagtaagatcgacaaaaatggcaaacgtacgttcgctgctacggtaattccaaaccgaggtgcgtggttggagtttgaaaccgatgcaaaggacgttgtctacgtaaggattgatcgcactcgcaagttgccgatcacggtgctgttgcgcgcgctcggtctttcttctgatcacgaaatcatcgacctgttgggcgaggatgagttcctgcagaacactctcgataaggatacgaccgattcgacggagcgtgctttggtagaaatctacgagcgtctgcgtcctggtgagccaccgacggtagaaaacgcccgggcgttgttggcttcgcgattctttgatccgaagcgttacgatttggcgagtgtcggtcggtataaaatcaacaagaaactgcacttgaaaaatcggcttttaaaccaacggctggctgagactctcgtcgacgcagatactggcgagattattgcggaagccggggcggtgctcgaccgacggacattggatcgcgtcattccgcatctgaacggcaatgttgggcgtttcacgattcgtgctacgcgcgatttgcttgaggatgaagttgtgcctctgcaaatgatcaagatttttagtccttctgaggatggtcgggtactgcacgtgatcagcaatggtgaattgccgtccgacgtgaagtacatcacgccgtccgacattcttgcggctgtgtcgtatttcttcaacttgttgcgcggggttggcacgacggacgacattgaccacttaggcaatcgtcgattgcgctctgtgggcgaactgttacagaaccaattccgcatcggtttgtcccgtatggaacgggttgtccgtgaacggatgtcgattcaagatgccagtgccatcacgccgcaggctctgatcaacattcgcccagtgatcgccgctattaaggagttcttcggttccagccagttgtcgcaatttatggatcagacgaacccgctggcggaattgacccataagcgtcgtctttcggctctgggaccgggcggtctgacgcgtgagcgcgctggtttcgaagttcgtgacgttcactactcacactacggccggatgtgtccgattgagacgccggaaggcccgaacatcggtctcatcaactcgttgtcgacgtatgcccggatcaatgagtatgggttcatcgaaacaccgtatcggcgagtggacccggaaacgggcgtcgtcaccgatcaaatcgactatttgacggccgatgaagaagagaattacgtcatcgcacaggccaacgagccgttgaccgaggatggtcgcctcgcatcggaagaaatcacggttcgatcccgcgaagacgtgatcacggtcaatcgcgatcgcattgattatatggacgtgtcgccgaaacaggttgtatcagttgcaacggctctgatcccgttcctcgaaaacgacgacgccaaccgtgctttgatgggatcgaacatgcagcggcaggcggttccgctgttggtgaccgattccccgtacgttggcacgggcatggaacatcaagcggcgaaagactctggtgtttgcgtcgtctcgcgccataacggcgtggttgagcgagctacagcccgtgaggtttgggttcgtcaggaagcggaagtcgacggcaagatcgtgaagggcgatctcgccaagtaccggttaattaagtttgcgagatcgaaccaaaacacatgcatcaaccagcggcccattgtacgcgagggacagcgggtcagggttggcgatatcctggcggacggcccggccacccagaaaggtgaactggccttaggccgcaacgtgcttgtcgcattcatgacgtgggagggctacaactacgaagacgccatcctgctgtccgagaagatggtgaaggaagacgtctatacgtcgattcacatcgaggagtacgaaatcgaggcgcgggatacgaagctgggaccagaggagattacgcgtgacatcccgaatgtcggcgaagatgctctgaaaaacctcgatgagcgcggtattatccgcatcggtgcggaaatccggacgggcgatatcctggtcggtaaggtgacgcccaaaggcgtgacggagctgactgcggaagagcgtctgttgcatgccatctttggtgagaaggcgcgtgaggtgcgcgatacgtcacttcgcgttccgcacggtggtgccggaattgtggtggacgtcaaggtgtttacccgtgagaacggggatgagttgcccgctggcgtcaatgagttagtacgggtctacatcgcgcagaagcgcaagatttccgaaggcgacaaaatggctggtcggcatggtaacaagggtgtcgtcgcacgcattctgccggaggaagacatgccgttcctcgaggatggaacgccggtcgaaatcgtactgaatccgctcggcgtgccgtctcgtatgaatatcggacaggtgcttgagacccacttgggtatggctgcaaaagtgctcggactgaaaatggcgacgccggtctttgacggtgcgcacccagaagatgtgtttgcgacgctgaaagaagctgggttgcccgaggacggcaagcaagttctgtacgacggccgcagtggcgagccgtttgagaaccgcgtcacggtgggttacgtctacatgttgaaattgcatcacctggtcgacgataagattcacgcgcgctcgaccggcccgtattccttggttacacagcagccgttgggcgggaaggcgcagtttggcggccaacggttcggcgagatggaggtatgggcgctcgaagcgtatggcgctgcatacaccttgcaggagattctcacggtcaagtccgacgacgtggtcggtcgcgtgaagacatacgaagccatcgtgaagggcgaaaacgtaccggaaccgggtgtgccggagtcgttcaaggttttgatcaaagagttgcagagccttggtatggacgtgaaaatcctcagtggcgacgagcgggaaatcgagatgaaggagatcgacgaggacgacgacggctcggaaaagctcaatttgaatctcgaatacaacgaagtcggcgattga

SEQ ID NO: 14 - DNA, 3531 bp, Alicyclobacillus hesperidum
  source 1..3531: mol_type = genomic DNA
ttgcagggacatatggtcaagtacggatgggcggagcgtcgtagctactcgcgcattcgtgaagtcctcgatcttcccaacctgattgaaatccagcaaaaatcgtatgaatggttcttgcgtgaaggactccgtgaaatgtttgccgacatttccccgattcaggacttcacgggcaatctagtgctcgaattcatcgactacagtctcggcgagccgaaatatgacgtcgaagagtcgaaggaacgcgatgtcacgtatgctgctccgcttcgtgtcaaggtgcgtttgctgaataaagagaccggggaagtgaaggaacaggaagtgttccttggtgactttccgctgatgacagacacgggaacgttcattatcaacggagccgagcgcgtgattgtcagccagttggtccggtcgcctagtgtctattatagcagtaagatcgacaaaaatggcaaacgtacgttcgctgctactgtaattccaaaccgcggtgcttggttggaattcgaaaccgatgcgaaggacgtcgtctatgtcaggatcgaccgtacgcgcaaattgccgatcacggtgttgctgcgtgcgcttggcctttcgtctgatcacgaaatcatcgacctgttgggtgaggatgagtacctgcagaacactctcgataaagacacgaccgattcgacggaacgagctttggtggagatctacgagcgcctgcgaccgggcgagccgccgacggtggaaaatgcccgggcgttgttggcgtcgcgtttctttgatccaaagcgctacgatttggcgagtgtcggtcgctataaaatcaataagaaactgcatttgaaaaaccggcttttaaatcaacggttggctgagacgctcgtcgatgcggacaccggtgagatcattgcagaggcaggggcggtgctcgatcgccggacgttggaccgggtgattccgcatttgaacggcaacgtgggccgtttcacgattcgtgccacacgggatctgcttgaggacgaagttgtgccgttgcagatgatcaagatttttagcccatccgaggacgggcgcgtattgcatgtgatcagcaacggtgaattgccgtctgacgtgaaatacatcacgccgtccgatatcttggcggctgtgtcgtatttctttaacttgttgcgtggggttggtacgactgacgatatcgatcatttggggaatcgtcgtctgcgctccgttggcgagttgctacagaatcagtttcgcatcgggttgtcgcgcatggaacgggtcgttcgtgagcgaatgtccattcaggatgcgagtgctatcactccacaggcactgatcaacattcgtccggtgatcgcggctattaaggaattctttggttctagccagttgtcacaatttatggatcaaaccaacccgttggcggagttgacgcacaagcgtcgtctgtctgccttggggccgggtggcttgacgcgcgagcgtgccggtttcgaagtgcgcgacgttcactattcccactacgggcgcatgtgtccgattgaaactcccgaaggcccaaacatcgggcttatcaactcgttatcgacgtatgcccggatcaatgagtatggctttattgaaacgccgtatcgtcgagttgatcctgagaccggtgtcgtcacggatcaaattgattatttgacggctgatgaggaagagaactatgtcattgcgcaggcaaacgagccgctgacagaagatggccgtctcgcttcggaagaaatcacggttcgatcccgcgaagacgtgattacggtcaatcgggatcgcatcgattatatggacgtgtctccgaagcaagtcgtgtcggttgcgacggctttgatcccgttcctcgaaaatgacgatgcgaaccgtgctttgatgggctcgaacatgcaacgccaagcggttccgctgttggtgaccgactcgccgtatgtcggcacaggtatggaacatcaggctgcgaaagactccggcgtttgcgtcgtctcgcgtcataacggtgtggtcgaacgcgcgacggctcgtgaggtctgggtccgccaagaagcggaagtcgatggcaaggtcgtcaagggcgatctcgctaagtaccggttgattaagtttgcgagatcgaaccaaaacacatgcattaaccagcggccaattgtgcgtgagggccagcgagtcaaggttggcgatattttggccgatggacctgcaactcagaacggcgagctggcgcttggacgcaatgtgcttgtcgcgttcatgacgtgggagggctacaactacgaagacgccatcctgctatctgaaaagatggtcaaggaagacgtctatacgtcaattcacatcgaggagtacgaaatcgaggcgcgcgatacgaagttggggccagaggagatcacgcgtgacattccgaatgtcggcgaagatgctctgaaaaacctcgacgagcgcggcatcatccggattggcgcagaaatccgcacgggcgatatcttggttggtaaagtgacgcccaaaggcgtgacggagctgacagcggaagaacgcttgttgcatgccatcttcggagaaaaggcgcgcgaggtgcgcgatacctcgttgcgcgtcccacatggtggcgccgggatcgtggtggatgtcaaagtgttcacccgtgaaaatggggacgaactgccggcgggtgtcaatgaactggtgcgcgtgtacatcgcacagaaacgcaagatttcggaaggcgacaaaatggctggtcgacatggtaacaagggtgttgtcgcccgcattttgccggaggaagacatgccgttcctagaagatggaacaccggtcgaaatcgtgctgaatccgctcggcgttccgtcccgtatgaatatcggacaggtgcttgagactcacttgggcatggcagcgaaggtgctcgggctgaaaatggcgacgcctgtcttcgatggggcgcatcctgaagatgtgttttcgacgctgaaagaagctgggttgcctgaggatggaaagcaggttctgtacgacgggcgcagcggtgagccatttgagaaccgtgttacggttggctacgtctatatgctgaagttgcaccacctcgttgacgataagattcatgctcgttcgacgggcccgtattctttggtgactcagcaaccgttgggtggtaaggcgcagttcggtggccagaggttcggtgagatggaggtttgggcgctcgaagcgtatggcgctgcatacaccttgcaagaaattctcacggtcaaatccgacgacgtcgttggtcgcgtaaagacgtacgaagccattgtgaagggtgaaaacgtaccggaaccaggcgtgccggagtctttcaaggtcttgatcaaggagctgcaaagccttggtatggatgtgaagatcctcagtggtgacgaacgcgagatcgagatgaaggagatcgacgaggacgacgacgggtcggaaaagctcaacttgaatctcgaatacaacgaagtcggcgattga

SEQ ID NO: 15 - DNA, 1959 bp, Alicyclobacillus contaminans
  source 1..1959: mol_type = genomic DNA
atgcattgccttccttttgcatgtttagaccaggatcttcagcttggagggaactcagtggcggaacaagtctacgatgaatcacaaatccaggtgttagagggccttgaggccgttcgcaagcgtccaggcatgtacatcggatcgaccagcgcacgagggcttcaccatctggtgtgggagattgtcgacaacgccgtggacgaagccctggccggccgttgtacgaaaatcatcgttcaaatccacaaagacaacagcgtctcggtgattgacaacggcgccggcttccccgtcggcatccacccaaagaccgggcgtcccgctgtggagaccgcgctgaccgtgctccatgccggaggcaaattcggcggcggtggatacaaggtgtcaggaggcctgcacggcgtcggggcgtccgtggtgaacgcgctgtcggagtggctgaaagtggtggtgtaccgcgatggcaagatttatacgcaggagtatgagcgcggcaacgtcctgtacgacctgaaagtgattggcgagacggatcgaaccgggaccacggtcaccttcaagcccgatccggaaattttcacggaaaccaccgagttcagttcggacattctgcaaagccgtctccgcgaactggcgtttctcaacgcggggttggaaattgtctacgaagacgagcgagaggacaagcaggtgacgtactgctattccggcggtgtggcggcttttgtggagtacctgaacaagtccaaagacgttctgcatcccacaccggtctatgtgacgggcgttcgggacgacgtgacggttgagattgccctgcagtataacgacggctactcgcagacgctgttctccttcgccaacaacattcacacgcacgaaggcggatctcacgaatccgggttcaagagcgcattgacgcgggtgattaacgactatgcgcggaagaacaacctggtcaaggacaatgaggacaagttgaccggcgatgacgtgcgcgaaggactaacggcaattgtcagcgtcaaggtgccggagcctcagtttgaaggacagaccaagaccaagctggggaacagcgaggtccgtggcatcgtggagagtttgtttgccgacaaattgcagattttcttcgacgaaaatcccagcgtggcgcgtaagattgtcgaaaagtcggtgaccgcggcgcgcgctcgcgaagcggcgagaaaagcgcgcgagctgacgcgtcgcaaaaacgcgctcgaggtcagttcattgcccggaaaattggccgattgttcgtccaaggatgcgtccgtcagtgaactgtatctggtcgagggtgactccgccggcggatctgcgaaacagggacgggatcggaattttcaggcaatcctcccgctgcgcggtaaaatcatcaatgtggagaaggcccggttggataaaatcctgtccaaccaagaaattcgggccatcatcaccgcgttgggcacgggaattggcgacgatttcgacatcaccaaagcccggtatcacaaagtggtcatcatgaccgatgccgatgtggacggaagccacattcgcacgcttttgttgacgctgttttaccggttcatgaagccgttgatagacgctggctacgtgtatatcgcgcagccgccactctacaagatcacgaagggaaagtccattcgctacgcgtattccgaccgagagttggaagccgttctccaggaagtggggcggcaaggggtcaacattcagagatacaaaggtctcggtgaaatgaatccgagccagctttgggaaaccaccatggatccggaggcccgcaccctgctacaggtcaccatggaagacgcgatggaagcagatatgattttcaccacgctgatgggtgataaggtggaaccgcgccgggagttcatcgaggaacacgcgcagtttgttcgcaacctggatatttaa

SEQ ID NO: 16 - DNA, 1917 bp, Alicyclobacillus acidoterrestris
  source 1..1917: mol_type = genomic DNA
ttggcggatacacgtacggaacaagtttatgatgagtctcagatagaagtcctggaaggcttgcaggctgttcgcaagcgtccgggcatgtatattggctccacgagtgtaaagggacttcaccacctcgtctgggaaattgtcgataatgcggtagacgaggcgctcgctgggcgttgtacgcgcattattgtgcaagttcacgaggacaacagtgtcacagttattgacaacggcgccgggtttcctgtgggaattcacccaaagaccggtcggccggccgtcgagacggcactgaccgtgcttcatgcaggcggtaaatttggcggcagcggctacaaggtgtcgggcgggcttcatggcgtcggcgcatcggtcgtcaatgcgttgtcttcgtggctcaaagttgaagtctggcgcgacgggcacgtatacgtgcaagagtacgaacgcggtgttcccaagtacgatttgaaagtggttggcgatactgataaacacggtacgagtgtcacgttcctgccagatccggagatttttacagagacgacggtgtttcagattgaaacactgcagcatcgccttcgcgagttggctttcctcaacgcaggattggcgattgtgctggaagatcagcgcgaaggtggcaagacgcaagaatttcattacgagggcggcgtcaaggaattcgtcagctatttaaataaatccaaagatgtcttgcatccagagccggtgtttgtcgaaggtgtcaaggacgatgtcaccatcgaagtggccctgcagtacaacgacggctatgcttcaacgatttactcgttcgccaacaacatcaacacaggtgaaggcgggacgcacgaatctggctttaagagcgcgttgactcgcgtgattaacgattacggccgcaagaacaacctgattaaggggagcgagagcaacctgacaggggacgatgtccgcgaggggattgtcgctgtcgtcagtgtgaaagttccggagccgcaatttgaagggcagaccaagacaaagcttggcaacagcgaggttcgcggtatcgtcgagagtctctttggcgacaagttgcagacattcctcgacgagaatccatcggttgcgcgcagaattatcgacaaatgtgtcaccgcggcacgtgcgcgggaagcggcacgcaaggctcgcgagttgacgcggcgcaaaagtgcactggaagtcagcagtctgccgggtaaattgaccgactgtgtgtcgaaagatccagagcgatcggaaatttacctggtggagggtgactccgcaggtggatcagcgaagatggggcgggatccacagacgcaagccattcttccactgcgcgggaaaattatcaatgtggagaaggcccggctggacaaaatcttgtctaacgaggaaattcgcgcaattatcacagctgcgggcaccggtattggtgacgaatttgatctctcgaaggcgcgttaccacaaggtggtcattatgacggatgccgatcacgatggcagccacatccgcattctgctattgacgttgttttatcgctttatgcggccgttgattgatgcgggttacgtgtatatcgcgcagcctccgctttacaagatctcaaaggggaaaacggttcgctatgcgtattctgacgcgcaactcgaagccattttgcaggagactggccgtcagggtgtcggcatccaacgatacaaaggtctcggtgagatgaacccggagcagttgtgggagaccacgatggatccggaatctcggaccctgctacaagtgacgatggaagacgctatggatgcggatatgatcttcagtacgctaatgggggataaagtggaaccgcgccgcgatttcatcgcggaacacgcgcgttatgtccgcaacctggacatctaa

SEQ ID NO: 17 - DNA, 1170 bp, Alicyclobacillus acidoterrestris
  source 1..1170: mol_type = genomic DNA
ggcagcggctacaaggtgtctggcgggcttcatggcgtcggcgcatcggtcgtcaatgcgttgtcttcgtggctcaaagtggaagtttggcgcgacgggcacgtatacgtgcaagagtacgaacgcggcgttcccaagtacgatttgaaagtggttggagataccgacaagcacggtacgagtgttacgttcctgccggatccggagatttttacagagacgacggtgtttcagattgaaacgctgcagcatcgccttcgcgagttggcctttctcaacgcgggattggcgattgtgctggaagatcagcgcgaaggtggcaagacgcaggaatttcactacgagggcggcgtcaaggaattcgtcagctatttaaataaatccaaagatgtcctgcatccagacccggtgtttgtcgagggcgtcaaggacgatgtcaccatcgaagtggccctgcagtacaacgacggctatgcttcgacgatttactcgttcgccaacaacatcaacacaggtgaaggcgggacgcacgaatctggcttcaagagcgcgctgactcgcgtgattaacgattacggccgaaagaacaacctgattaaggggagcgagagcaacctgacaggggacgatgtccgcgaggggattgtcgctgtcgtcagcgtgaaagttccagagccgcaatttgaaggacagaccaagaccaagcttggcaacagtgaggttcgcggtatcgtcgagagtctctttggcgacaagctgcagacattcctcgacgagaatccatcggttgcgcgcaaaattatcgacaagtgtgtcaccgcggcacgcgcgcgggaagcggcgcgcaaggctcgcgagttgacacggcgcaaaagtgcactggaagtcagcagtctgccaggtaagttgaccgactgtgtgtcgaaggatccagagcgatcggaaatctatctggtggagggtgactccgcaggcggatcggcgaagatggggcgggatccacagacgcaagccattcttccgctgcgcggaaaaatcatcaatgtggagaaggcccggctggacaaaatcttgtccaacgaggaaatccgcgcgatcatcacagctgcgggcaccggtattggcgacgaattcgatctctcgaaggcgcgttaccacaaggtggtcatt

SEQ ID NO: 18 - DNA, 1917 bp, Alicyclobacillus acidoterrestris
  source 1..1917: mol_type = genomic DNA
ttggcggatacacgtacggaacaagtttatgatgagtctcagatagaagtcctggagggcttgcaggctgttcgcaagcgtccgggcatgtatattggctccacgagtgtaaagggacttcaccacctcgtgtgggaaattgtcgataatgcggtggacgaggcgctcgctggccgttgtacgcgcattattgtgcaagttcacgaggacaacagtgtcacagtcattgacaacggcgccgggtttcctgtggggattcacccaaagaccggtcggccggccgtcgaaacggcactgaccgtgcttcatgcaggcggtaaatttggcggcagcggctacaaggtgtctggcgggcttcatggcgtcggcgcatcggtcgtcaatgcgttgtcttcgtggctcaaagtggaagtttggcgcgacgggcacgtatacgtgcaagagtacgaacgcggcgttcccaagtacgatttgaaagtggttggagataccgacaagcacggtacgagtgttacgttcctgccggatccggagatttttacagagacgacggtgtttcagattgaaacgctgcagcatcgccttcgcgagttggcctttctcaacgcgggattggcgattgtgctggaagatcagcgcgaaggtggcaagacgcaggaatttcactacgagggcggcgtcaaggaattcgtcagctatttaaataaatccaaagatgtcctgcatccagacccggtgtttgtcgagggcgtcaaggacgatgtcaccatcgaagtggccctgcagtacaacgacggctatgcttcgacgatttactcgttcgccaacaacatcaacacaggtgaaggcgggacgcacgaatctggcttcaagagcgcgctgactcgcgtgattaacgattacggccgaaagaacaacctgattaaggggagcgagagcaacctgacaggggacgatgtccgcgaggggattgtcgctgtcgtcagcgtgaaagttccagagccgcaatttgaaggacagaccaagaccaagcttggcaacagtgaggttcgcggtatcgtcgagagtctctttggcgacaagctgcagacattcctcgacgagaatccatcggttgcgcgcaaaattatcgacaagtgtgtcaccgcggcacgcgcgcgggaagcggcgcgcaaggctcgcgagttgacacggcgcaaaagtgcactggaagtcagcagtctgccaggtaagttgaccgactgtgtgtcgaaggatccagagcgatcggaaatctatctggtggagggtgactccgcaggcggatcggcgaagatggggcgggatccacagacgcaagccattcttccgctgcgcggaaaaatcatcaatgtggagaaggcccggctggacaaaatcttgtccaacgaggaaatccgcgcgatcatcacagctgcgggcaccggtattggcgacgaattcgatctctcgaaggcgcgttaccacaaggtggtcattatgacggatgccgatcacgacggcagccacatccgcattctgctattgaccttgttctatcgctttatgcggccgttgattgatgcgggttacgtgtatatcgcgcagcccccgctttacaagatctcaaaggggaaaacggtccgctatgcgtattctgacgcgcaactcgaagccattttgcaggagactggccgtcagggtgtcggcatccaacggtacaaaggtctcggtgagatgaatccggagcagttgtgggagaccacgatggatccggaatctcggaccttgctgcaagtgacgatggaagacgccatggatgcggatatgatcttcagcacgctgatgggggataaagtggaaccgcgccgcgatttcatcgcggaacacgcgcgttatgtccgcaatctggacatctaa

SEQ ID NO: 19 - DNA, 1170 bp, Alicyclobacillus acidoterrestris; identical to SEQ ID NO: 17
  source 1..1170: mol_type = genomic DNA

SEQ ID NO: 20 - DNA, 1956 bp, Alicyclobacillus hesperidum
  source 1..1956: mol_type = genomic DNA
atgaacatccgagaggctttgctctgtcttgacgtgagtgtggagggaacccagttgtcagaacaagtgtacgacgagtcgcaaatagaagtcctggaaggcttgcaggctgtgcgaaaacgccctggtatgtacatcggatcgaccagtgttaaaggcctacaccatctggtctgggaaatcgtcgacaatgcggtcgacgaagcactggcggggcgatgcacgagaatcgtcgtccaggttcacccggataatagcgtcagtgttatagacaacggcgccgggtttcccgtcgggattcacgccaagacagggcggccggcggttgaaacggctttaacggtcctgcatgctggtgggaagtttggtggtgccggatataaagtgtcaggtggattgcacggcgtgggtgcatccgttgtaaatgctttatccgaatggctgaaggtggaggttcatcgcgacggcaaaatccacgtccaagaatacgagcgcggaactccgctttatgatttgaaagttgttggcgagacggatcggacaggcacgacggttacgttccggccggacccggagatttttacggagaccacggagttttccgcggagactttgcaaaaccgtttgcgggagctggcgtttttaaacgctgggctcgaaattgtgctcgaagatgaacgcgaaggtggcaagcgatttgagtatcactacgagggtggcgtcaaggagtttgtcctttacttgaaccattcaaaagacgttctgtttgctgagccaatttatgtacagggtgtcaaagatgaggtcacggtcgagattgccctacagtacaacgatggctatagctccaccttgtactcgtttgcgaacaacatcaacacaggtgaaggcggcacgcatgaagccggctttaaaagtgcactgacccgcattatcaacgactatggccgtcgcaataacctcatcaagggcagtgacggcagtttgacaggtgacgacgttcgcgaaggactgacggcggttgtcagcgtaaaggtcccggaaccgcaatttgagggacaaacaaagacaaagttgggcaatagcgaagtccgcgggatcgtggaaagtctgtttggcgaacggctacaaatctttctcgaggagaatccggccgtagctcggagaatcctcgaaaaatgcgtgctcgcggcgcgcgctcgagaagctgcgaaacgagcacgtgaacttactcgcaacaagaagatggaagtcacaagcttgcccggcaagctgacggactgcgtttcaaaggacccggaagtgtctgaactgtatttggttgagggcgactcagccggcggatcggcaaagggcgggcgagatccacaaacacaagccatcctgccgttgcgaggaaagattatcaatgtcgaaaaggcccgcttggacaaggtattgtccaacgaggaaattcgggccattatcaccgcatgtggcacagggattggcgacgaatttgatttgagtaaagcacgctatcacaaaatcgtcatcatgacggatgctgatcacgacggtagccacattcgcattttgctgttgaccttgttctatcggtttatgcgaccgcttatcgatgcaggctatatctatatcgcacaaccaccgctttacaaaatcgagaagggcaaagtgattcgctacgcgtactcggatacacaactggagcaagcgttacaagaaatgggccgacagggcgtgaaattgcaacggtataaggggctcggggaaatgaacgcagaccaactctgggagacgacgatggatccagagtcccgcaccctgctgcaagttaccatggaagacgccatggacgccgacatgatcttcagtaccttaatgggcgacaaggtcgaaccgcgacgagattttattgcagagcacgcgcgctatgtgcggaatctcgatgtatga

SEQ ID NO: 21 - DNA, 1902 bp, Alicyclobacillus sacchari
  source 1..1902: mol_type = genomic DNA
ttgtcagaacaagtgtacgacgagtcgcaaatagaggtcctggaaggcttgcaggctgtgcggaaacgccctggtatgtacatcggatcgaccagtgtcaaaggcctgcaccatctggtctgggaaattgtcgacaacgccgtcgacgaagcactggcggggcgatgcacgaggatcgtcgtccaggttcacccggataatagcgtcagtgtcatagacaacggcgccgggtttcccgtcgggattcacgccaagacagggcggcccgcggtggaaacggccttaactgtcctacacgcaggtggcaagtttggcggtgccggatataaagtatcaggtggtttgcacggtgtcggtgcatccgttgtgaacgctttgtctgaatggctgaaagttgaagtccatcgggacggcaagattcacgtacaagaatacgagcgcgggactccgctgtacgacttaaagattgtcggggagacgaatcgaacaggcacaactgtcacgttccggccggatccagagattttcacggaaacgacggagttttcagcggagacgctgcaaaatcgcttacgggaactggcgtttttaaacgccgggctcgagattgtgctcgaggatgagcgcgagggcgggaagcgtttcgagtaccactacgaaggtggtgtcaaagagttcgttctctatctgaaccactcaaaggacgtcttgtttcctgagccgatttacgtacagggcgtgaaagaagaggtcacggtcgagattgcattgcagtataacgacggctacagttcgaccttgtactcgtttgcaaacaacatcaacaccggcgagggtggtacccacgaagccggttttaagagcgcactgacccgtattatcaacgattatggtcgtcgcaataacctcgttaaaggcagtgacggcagtctgacaggcgatgacgtccgcgaagggcttacagccgttgtcagtgtgaaagttccggagccacaatttgagggacaaaccaagacaaagctgggtaacagcgaagtccgcgggatcgtcgaaagtctgttcggagagcggctacaaatcttcctcgaggagaatccgtctgtggctcgcaggattctcgaaaagtgtgtgcttgccgcacgtgcccgagaggcagctaaacgggctcgcgaacttactcgcaacaaaaagatggaagtcacgagtttgcctggaaagttgacggattgcgtttcaaaggacccagaagtctcggaattgtatctcgtcgagggtgattcggccgggggatcggcaaagggtgggcgagatccgcaaacacaagccatcttgccgctgcgggggaagattatcaacgtcgaaaaggcccgcttggacaaggtgttgtccaacgaagaaatcagggccattattaccgcatgtggcaccggtatcggcgacgagtttgatttgagcaaagcccgttatcacaaaatcgtcattatgacagatgccgatcacgacggcagccatattcgcatcctgctgttgacgttgttctatcgctttatgcggccgctcatcgatgccggctatatctatatcgcacaaccgccactctacaagattgagaagggcaaagtgattcgctacgcctactccgatgcgcaactggagcaagcgttgcaagaaatgggcagacagggcgtgaagttgcagcggtacaagggcctcggggaaatgaatgccgaccaactctgggaaacgacgatggacccagagtctcgtacattgctgcaagtcacgatggaagacgcgatggacgccgacatgatctttagcaccttgatgggcgacaaggtcgaaccacgccgagatttcattgcagagcacgcgcgttatgtgcggaacctcgacgtgtga

SEQ ID NO: 22 - DNA, 1899 bp, Alicyclobacillus acidiphilus
  source 1..1899: mol_type = genomic DNA
ttgtcagaacaagtgtatgacgaatctcagatagaagtccttgaaggcctgcaggctgtccgcaaaagaccaggtatgtacattgggtcgacaagcgtaaaaggcttgcaccacctggtatgggaaattgtcgacaatgcggtcgacgaagccttggcgggcaggtgtacgaaaattatcgtgcaggtacatgaggacaacagcgtcagcgtgatcgacaacggggcaggctttccagtcggcattcacgcgaaaacgggacgtcctgcagttgagacggcccttacggttctgcatgcgggcggcaaattcggcggcgcaggctacaaagtttccggcggcctgcacggcgtcggcgcttcggtcgtcaacgctttgtcgatctggctgcgggtggaagtgcaccgtgatggacaagtgtacgagcaagagtatgagcgcggcgtgccgttgtacgacttgcgcgtcattggcaccacggatcggacaggaacgacggtcagcttccggccggatcctgaaatctttacggaaaccacggaattttcgtttgaaacactgcagggccgtttgcgcgagctcgccttcctcaacgcggggctgaacattattctcgaagacgaacgagacggaaagcgcgtggattaccggtacgaaggcggcgtcagcgagtttgtgcgtttcctgaaccagtccaaagatgtgctgttcccggatcccgtcttcgtctccggcgtcaaggacgatgtcacagtcgagatcgcgctgcagtacaacgacgggtacagctcgacgctctattcgttcgccaacaacatcaatacgggcgaaggcggaacgcacgaagccggatttaagagcgcgctcacgcgtgtgatcaacgattacggccggaagaacaatctgctcaagagcaacgacggatcattgaccggcgacgacgtgcgcgaaggactgactgccgtcatcagcgtgaaagtgccagaaccgcagttcgaaggacagaccaagacgaagctgggcaacagtgaggttcgcggcatcgtggagagtctgttcggtgaacacctgcagattttcctcgaagaaaatccgacggtggcgcgccgcatcctcgacaagagcgtgatggccgcacgggcccgagaagccgcgaaacgcgctcgcgagctgacgcgaagcaagaaaatggacgtcacgagccttccgggcaaactgaccgactgtgtgtccaaagaccctgagatgtccgagttgtatctggtcgagggcgattccgcgggcggttctgccaagggcggacgggatccgcagacgcaggccattctcccgcttcgcggcaagatcatcaacgtggagaaggctcgactggacaagatcttgtccaacgaagaaattcgcgccatcatcacagcgtgcggcaccggtatcggcgacgagttcgatatcacgaaagcgcggtatcacaagattgtgatcatgacggatgccgatcacgacggcagtcacatccgcattcttctgctgacgttgttctatcggtttatgcggccgctcatcgacgccggatatatctacatcgcacagccgcctttgtacaagatcacgaagggcaaagtcgttcgctacgcctactcggacgcgcagctcgaaacggccttgcaggagatggggcgccagggcgttggacttcagcgctacaaaggtcttggcgagatgaacgcggaccagctttgggagacgaccatggacccagattcgcggacgctgcttcgcgtgacgatggaagacgccatggacgccgatatgatcttcagcacgttgatgggtgacaaagtggagccgcggcgcgatttcatcgcggaacatgcccggtacgttcggaacctcgacgtgtaa

SEQ ID NO: 23 - DNA, 1164 bp, Alicyclobacillus acidiphilus
  source 1..1164: mol_type = genomic DNA
ggcgcaggctacaaagtttccggcggcctgcacggcgtcggcgcttcggtcgtcaacgctttgtcgatctggctgcgggtggaagtgcaccgtgatggacaagtgtacgagcaagagtatgagcgcggcgtgccgttgtacgacttgcgcgtcattggcaccacggatcggacaggaacgacggtcagcttccggccggatcctgaaatctttacggaaaccacggaattttcgtttgaaacactgcagggccgtttgcgcgagctcgccttcctcaacgcggggctgaacattattctcgaagacgaacgagacggaaagcgcgtggattaccggtacgaaggcggcgtcagcgagtttgtgcgtttcctgaaccagtccaaagatgtgctgttcccggatcccgtcttcgtctccggcgtcaaggacgatgtcacagtcgagatcgcgctgcagtacaacgacgggtacagctcgacgctctattcgttcgccaacaacatcaatacgggcgaaggcggaacgcacgaagccggatttaagagcgcgctcacgcgtgtgatcaacgattacggccggaagaacaatctgctcaagagcaacgacggatcattgaccggcgacgacgtgcgcgaaggactgactgccgtcatcagcgtgaaagtgccagaaccgcagttcgaaggacagaccaagacgaagctgggcaacagtgaggttcgcggcatcgtggagagtctgttcggtgaacacctgcagattttcctcgaagaaaatccgacggtggcgcgccgcatcctcgacaagagcgtgatggccgcacgggcccgagaagccgcgaaacgcgctcgcgagctgacgcgaagcaagaaaatggacgtcacgagccttccgggcaaactgaccgactgtgtgtccaaagaccctgagatgtccgagttgtatctggtcgagggcgattccgcgggcggttctgccaagggcggacgggatccgcagacgcaggccattctcccgcttcgcggcaagatcatcaacgtggagaaggctcgactggacaagatcttgtccaacgaagaaattcgcgccatcatcacagcgtgcggcaccggtatcggcgacgagttcgatatcacgaaagcgcggtatcacaagattgtgatc

SEQ ID NO: 24 - DNA, 1914 bp, Alicyclobacillus acidocaldarius
  source 1..1914: mol_type = genomic DNA
ttggcggatcgaataccggaacaggtgtacgatgagtcgcaaattgaggtcctggaaggactccaggccgtgcgcaagcggcctggcatgtatatcggctcgacgagcgcgaaggggctgcaccaccttgtgtgggagattgtcgacaacgcggtcgatgaagcgctcgccgggcgatgcacgcggatcgtggtgcaggtccacccggacaacagcgtgacggtcatagacaacggggccgggtttcccgtgggcattcacccgaagaccggaaggcccgcggtcgagacggtcttgaccacgctgcacgcgggcggcaagttcggcggcaaagggtacaaggtttcgggcggcctgcatggcgtgggtgcctccgtggtgaacgcgctctcggagtggatgcgcgtcgaagtgcatcgcgacggccgcatttatgtgcaggagtacgagcgcggcacgccgctgtacgacctgaaggtcattggcacgacggacaagacggggacgaaggtctcgttcaagccggatcccgagatcttcacggagacgacggtgttctcggcggagacgctcatgaaccgcctgcgcgagctcgccttcctcaacgcgggcctggagattgtgttcgaggacgagcgggagggcggcaagcgggcggtcttcaagtacgacgggggcgtcgcggaattcgtccgctggctaaatcagtcgaaggacgtgctgtttgacgatcccatctacgtctccgcggtgaaggacgacgtcgcggtcgagatcgccctccaatacaacgacggctatgcagcaacggtctactcgttcgcgaacaacatcaacacggcggagggcggcacgcacgaggccggcttcaagagcgcgctgacgcgcgtgatcaacgactacgctcggcgatacggcttcctgaagtcgaacgacaacagcatgaccggcgacgacgtccgcgaggggctgaccgccgtggtgagcgtcaaggttcctgagccgcagttcgagggccagaccaagacgaagctcggcaacagcgaggtccgcggcatcgtcgagacgctcttcgccgagcggatgcagatgttcctcgaggagaacccgtccatcgcgcggaagattgtggagaagtgcattctcgcggcgagggcgcgcgaggcggcaaggcgcgcgcgcgagttgacgcgcagcaagaagatggacgtgacgagcctccccggcaagctcaccgactgcagctcgaaggatccggagcggtccgagctgtttctcgtcgaaggagattccgcaggcggatcggccaagatggggcgagatccgcagacgcaggccattctgccgcttcgcgggaagatcatcaacgtcgaaaaggcgcgcttggacaaggtgctggcgaacgaggagattcgcgccatcatcaccgcgtgcggcacgggcatcggcgacgacttcgatatttcgaaggcccgctatcacaagatcgtcatcatgacggatgcggatcacgacgggagccacattcgcattctgttgctcacgctcttctaccggttcatgcggccgctcatcgacgcgggctacatctacatcgcccagccgccgctttacaagatcgagaagggcaaagtggtgcggtacgcctattccgacgcccagctcgagcagatccttcaggagatgggccgtgagggcgtgcggctgcagcggtacaaggggctcggcgagatgaacgccgaccagctctgggagacgacgatggatccggagtcgcgcacgctgctccgcgtcacgatggaggatgcgatggatgcggacatgatcttcagcatcctcatgggcgacaaggtcgagccgcggcgggatttcatcgcggaacacgcgcgatttgtccgcaacttggacgtgtga

SEQ ID NO: 25 - DNA, 1914 bp, Alicyclobacillus sendaiensis
  source 1..1914: mol_type = genomic DNA
ttggcggatcgaataccggaacaggtgtacgatgagtcgcaaattgaggtcttggaagggcttcaggccgtgcgcaagcggcctgggatgtatatcggctcgacgagcgcgaaggggctgcaccaccttgtgtgggagatcgtcgataacgcggtggatgaagcgctcgccgggcgatgcacgcgcatcgtggtgcaggttcacccggacaacagcgtgacggtcgtagacaacggggccgggtttcccgtgggcattcatccgaagacgggcaggcccgcggtcgagaccgtcttgaccaccctgcacgccggcggcaagttcggcggcaaggggtacaaggtttccggcggcctgcacggcgtgggcgcctcggtcgtcaacgcactgtcggagtggatgcgcgtcgaagtgcatcgcgacggacacatttatgtccaggagtacgagcgcggcacgccgctttacgacctgaaagtgataggcacgacggacaagaccggaacaaaggtctcgttcaagccggatcccgagatcttcacagagacgacggtgttttcggcagagaccctcatgaaccgcctgcgcgaactcgcctttctcaacgcggggctcgagattgtgttcgaagatgagcgggagggcggcaagcgcgccgtgttcaagtacgacggcggcgtcgccgagttcgtccgctggctgaatcagtcgaagaacgtgctgtttgacgatcccatctacgtctccgcggtcaaggatgacgtggcggtcgagatcgccctccagtacaacgacggctacacggcgacggtgtactcgttcgcgaacaacatcaacacggtagagggcggcacgcacgaggcgggcttcaagagcgcgttgacgcgcgtcatcaacgactacgcgaggcgacacggctttttgaagtcgaacgacaacagcatgaccggggacgatgtccgcgaagggctgacggccgtggtcagcgtcaaggttcccgagccccagttcgagggccagacgaagacgaagctcggcaacagcgaggtgcgcggcatcgtcgagacgctcttcgccgagcggatgcagatattcctcgaggagaacccggccatcgctcgcaagatcgtcgataagtgcattcttgcggcgcgggcgcgcgaagcggcgaggcgggcgcgggagctgacgcgaagcaagaagatggacgtgacgagcctgcccggcaagctgacggactgcagttcgaaagatcccgagcggtcggagttgttcttggtggagggcgattccgcgggcggatcggccaagatggggcgagatccgcagacgcaggcgattctgccgctgcgcgggaagatcatcaacgtggaaaaggcgcgactggacaaggtccttgcgaacgaggagatccgcgccatcatcacggcgtgcggcacgggcatcggagacgacttcgacatctcgaaggcccgctaccacaagattgtcatcatgaccgatgccgatcacgacgggagccacatccgcatcctgttgctcacgctcttctaccggttcatgcggccgctcatcgacgcgggctatatctacatcgcccagccgccgctgtacaagatcgagaagggcaaggtggtccgctacgcctattccgatgcgcagctggagcagatcctgcaggagatggggcgcgacggcgtgcggctacagcggtataaagggctcggtgagatgaacgcagatcagctgtgggagacgacgatggatcccgagtcgcgcacgctgcttcgcgtcaccatggaggacgcgatggacgcggacatgatcttcagcatcctcatgggggacaaggtggagccgcggcgggacttcatcgcggaacacgcgcggttcgtgcgcaacttggacgtctga

SEQ ID NO: 26 - DNA, 1167 bp, Alicyclobacillus sendaiensis
  source 1..1167: mol_type = genomic DNA
ggcaaggggtacaaggtttccggcggcctgcacggcgtgggcgcctcggtcgtcaacgcactgtcggagtggatgcgcgtcgaagtgcatcgcgacggacacatttatgtccaggagtacgagcgcggcacgccgctttacgacctgaaagtgataggcacgacggacaagaccggaacaaaggtctcgttcaagccggatcccgagatcttcacagagacgacggtgttttcggcagagaccctcatgaaccgcctgcgcgaactcgcctttctcaacgcggggctcgagattgtgttcgaagatgagcgggagggcggcaagcgcgccgtgttcaagtacgacggcggcgtcgccgagttcgtccgctggctgaatcagtcgaagaacgtgctgtttgacgatcccatctacgtctccgcggtcaaggatgacgtggcggtcgagatcgccctccagtacaacgacggctacacggcgacggtgtactcgttcgcgaacaacatcaacacggtagagggcggcacgcacgaggcgggcttcaagagcgcgttgacgcgcgtcatcaacgactacgcgaggcgacacggctttttgaagtcgaacgacaacagcatgaccggggacgatgtccgcgaagggctgacggccgtggtcagcgtcaaggttcccgagccccagttcgagggccagacgaagacgaagctcggcaacagcgaggtgcgcggcatcgtcgagacgctcttcgccgagcggatgcagatattcctcgaggagaacccggccatcgctcgcaagatcgtcgataagtgcattcttgcggcgcgggcgcgcgaagcggcgaggcgggcgcgggagctgacgcgaagcaagaagatggacgtgacgagcctgcccggcaagctgacggactgcagttcgaaagatcccgagcggtcggagttgttcttggtggagggcgattccgcgggcggatcggccaagatggggcgagatccgcagacgcaggcgattctgccgctgcgcgggaagatcatcaacgtggaaaaggcgcgactggacaaggtccttgcgaacgaggagatccgcgccatcatcacggcgtgcggcacgggcatcggagacgacttcgacatctcgaaggcccgctaccacaagattgtcatc

SEQ ID NO: 27 - DNA, 1550 bp, synthetic construct
  misc_feature 1..1550: note = synthetic construct
  source 1..1550: mol_type = other DNA
ttatcggagagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcgtaacaaggtagccgtatcggaaggtgcggctggatcacctccttt

SEQ ID NO: 28 - DNA, 1511 bp, synthetic construct
  misc_feature 1..1511: note = synthetic construct
  source 1..1511: mol_type = other DNA
gacgaacgctggcggcgtgcctaatacatgcaagtcgagcggaccggattggggcttgccttgattcggtcagcggcggacgggtgagtaacacgtgggcaacctgcccgcaagaccgggataactccgggaaaccggagctaataccggataacaccgaagaccgcatggtcttcggttgaaaggcggcctttgggctgtcacttgcggatgggcccgcggcgcattagctagttggtgaggtaacggctcaccaaggcgacgatgcgtagccggcctgagagggtgaccggccacactgggactgagacacggcccagactcctacgggaggcagcagtagggaatcttccgcaatgggcgaaagcctgacggagcgacgccgcgtgagcgaagaaggccttcgggtcgtaaagctctgttgtgagggacgaaggagcgccgttcgaagagggcggcgcggtgacggtacctcacgagaaagccccggctaactacgtgccagcagccgcggtaatacgtagggggcgagcgttgtccggaattattgggcgtaaagcgcgcgcaggcggtctcttaagtctgatgtgaaagcccacggctcaaccgtggagggtcattggaaactgggggacttgagggcaggagaggagagcggaattccacgtgtagcggtgaaatgcgtagagatgtggaggaacaccagtggcgaaggcggctctctggcctgcacctgacgctgaggcgcgaaagcgtggggagcaaacaggattagataccctggtagtccacgccgtaaacgatgagtgctaagtgttagaggggtcacaccctttagtgctgcagctaacgcgataagcactccgcctggggagtacggccgcaaggctgaaactcaaaggaattgacgggggcccgcacaagcggtggagcatgtggtttaattcgaagcaacgcgaagaaccttaccaggtcttgacatcccctgacaacccaagagattgggcgttcccccttcggggggacagggtgacaggtggtgcatggttgtcgtcagctcgtgtcgtgagatgttgggttaagtcccgcaacgagcgcaaccctcgcctctagttgccagcattcggttgggcactctagagggactgccggcgacaagtcggaggaaggtggggatgacgtcaaatcatcatgccccttatgacctgggctacacacgtgctacaatgggcggtacaaagggctgcgaacccgcgagggggagcgaatcccaaaaagccgctctcagttcggattgcaggctgcaactcgcctgcatgaagccggaatcgctagtaatcgcggatcagcatgccgcggtgaatacgttcccgggccttgtacacaccgcccgtcacaccacgagagcttgcaacacccgaagtcggtgaggtaacccgcaagggagccagccgccgaaggtggggcaagtgattggggtgaagtcgtaacaaggtagccgtaccggaagg

SEQ ID NO: 29 - DNA, 1446 bp, synthetic construct
  misc_feature 1..1446: note = synthetic construct
  source 1..1446: mol_type = other DNA
tggcggcgtgcctaatacatgcaagtcgagcggatgtgatgaagagcttgctcttctgaacattagcggcggacgggtgagtaacacgtgggtaacctggctgtaagactgggataaccccgggaaaccggagctaataccagataataagatttctcgcatgagagatttttgaaaggtgctaaggcatcacttacaaatgggcccgcggcgcattagctagttggtggggtaaaggcctaccaaggcaacgatgcgtagccgacctgagagggtgaccggccacactggaactgagacacggtccagactcctacgggaggcagcagtagggaatcttccgcaatggacgaaagtctgacggagcaacgccgcgtgagcgaagaaggtcttcggattgtaaagctctgtctttaaggaagaacagctatgagagggaatgctcatagagtgacggtacttaaggaggaagccccggctaactacgtgccagcagccgcggtaatacgtagggggcaagcgttgtccggaattattgggcgtaaagcgcgcgcaggtggccatgtaagtctgatgtgaaattctagggctcaaccttagaactgcattggaaactgcatggcttgagagcaggagagggaagtggaattcctagtgtagcggtgaaatgcgtagatattaggaggaacaccagtggcgaaggcgacttcctggcctgttactgacactgaggcgcgaaagcgtggggagcaaacaggattagataccctggtagtccacgctgtaaacgatgagtgctatgtgttggagggtaccaccttcagtgcagtagttaacgcaataagcactccgcctggggagtacggtcgcaaggctgaaactcaaaggaattgacggggacccgcacaagcagtggagcatgtggtttaattcgaagcaacgcgaagaaccttaccaaggcttgacatccctctgaccggtgtagagatacacctttccttcgggacagaggtgacaggtggtgcatggttgtcgtcagctcgtgtcgtgagatgttgggttaagtcccgcaacgagcgcaacccctattattagttgccagcattaagttgggcactctaatgagactgccggtgacaaaccggaggaaggcggggatgacgtcaaatcatcatgccccttatgtcttgggctacacacgtgctacaatggatggtacagagggaagcgaagccgcgaggtggagccaatcccaaaaagccattctcagttcggattgtaggctgcaactcgcctacatgaagctggaattgctagtaatcgcggatcagaatgccgcggtgaatacgttcccgggtcttgtacacaccgcccgtcacaccacgagagtttgtaacacccgaagccggtggggtaacccgcaagggagctagccgtcgaaggtgg

SEQ ID NO: 30 - DNA, 1520 bp, synthetic construct
  misc_feature 1..1520: note = synthetic construct
  variation 182
  variation 192
  variation 476
  source 1..1520: mol_type = other DNA
tagagtttgatcatggctcaggacgaacgctggcggcgtgcctaatacatgcaagtcgagcggacggaagggagcttgctcctggaagtcagcggcggacgggtgagtaacacgtgggtaatctgcctgtcagaccggaataacgcctggaaacgggtgctaatgccggatagtcagtcttngggcatcttnggactgggaaaggcgctaaggcgtcactgacagaggagcccgcggcgcattagctagttggtggggtaaaggcctaccaaggcgacgatgcgtagccgacctgagagggtgaccggccacactgggactgagacacggcccagactcctacgggaggcagcagtagggaatcttcggcaatgggcgaaagcctgaccgagcaacgccgcgtgagcgaagaaggccttcgggttgtaaagctcagtcactcgggaagagcgaaccggagagggaatgctctggtngagacggtaccgagagaggaagccccggctaactacgtgccagcagccgcggtaatacgtagggggcgagcgttgtccggaatcactgggcgtaaagcgtgcgtaggcggtttggtaagtccggagtgaaagaccagggctcaaccctgggggtgctttggaaactgccagacttgagtgctggagaggcaaggggaattccacgtgtagcggtgaaatgcgtagagatgtggaggaataccagtggcgaaggcgccttgctggacagtgactgacgctgaggcacgaaagcgtggggagcaaacaggattagataccctggtagtccacgccgtaaacgatgagtgctaggtgttggggggtaccaccctcagtgccgaaggaaacccaataagcactccgcctggggagtacggtcgcaagactgaaactcaaaggaattgacgggggcccgcacaagcagtggagcatgtggtttaattcgaagcaacgcgaagaaccttaccagggcttgacatccctctgacgggttcagagatgagccgtcccttcggggcagaggagacaggtggtgcatggttgtcgtcagctcgtgtcgtgagatgttgggttaagtcccgcaacgagcgcaacccttgacctgtgttaccagcacgtgaaggtggggactcacaggtgactgccggcgtaagtcggaggaaggtggggatgacgtcaaatcatcatgccctttatgtcctgggctacacacgtgctacaatgggcggtacaacgggaagcgagaccgcgaggtggagcgaaaccctgaaagccgttcgtagttcggattgcaggctgcaactcgcctgcatgaagccggaattgctagtaatcgcggatcagcatgccgcggtgaatccgttcccgggccttgtacacaccgcccgtcacaccacgagagttggcaacacccgaagtcggtgaggtaaccctttagggagccagccgccgaaggtggggtcgatgattggggtgaagtcgtaacaaggtagccgtatcggaagg

SEQ ID NO: 31 - DNA, 1472 bp, synthetic construct
  misc_feature 1..1472: note = synthetic construct
  variation 158
  variation 161..162
  variation 171
  variation 299
  variation 455
  source 1..1472: mol_type = other DNA
gacgaacgctggcggcgtgcctaatacatgcaagtcgagcggacggaagggagcttgctcctggaagtcagcggcggacgggtgagtaacacgtgggtaatctgcctgtcagaccggaataacgcctggaaacgggtgctaatgccggatagtcagtnttnnggcatcttnggactgggaaaggcgctaaggcgtcactgacagaggagcccgcggcgcattagctagttggtggggtaaaggcctaccaaggcgacgatgcgtagccgacctgagagggtgaccggccacactgggantgagacacggcccagactcctacgggaggcagcagtagggaatcttcggcaatgggcgaaagcctgaccgagcaacgccgcgtgagcgaagaaggccttcgggttgtaaagctcagtcactcgggaagagcgaaccggagagggaatgctctggtngagacggtaccgagagaggaagccccggctaactacgtgccagcagccgcggtaatacgtagggggcgagcgttgtccggaatcactgggcgtaaagcgtgcgtaggcggtttggtaagtccggagtgaaagaccagggctcaaccctgggggtgctttggaaactgccagacttgagtgctggagaggcaaggggaattccacgtgtagcggtgaaatgcgtagagatgtggaggaataccagtggcgaaggcgccttgctggacagtgactgacgctgaggcacgaaagcgtggggagcaaacaggattagataccctggtagtccacgccgtaaacgatgagtgctaggtgttggggggtaccaccctcagtgccgaaggaaacccaataagcactccgcctggggagtacggtcgcaagactgaaactcaaaggaattgacgggggcccgcacaagcagtggagcatgtggtttaattcgaagcaacgcgaagaaccttaccagggcttgacatccctctgacgggttcagagatgagccgtcccttcggggcagaggagacaggtggtgcatggttgtcgtcagctcgtgtcgtgagatgttgggttaagtcccgcaacgagcgcaacccttgacctgtgttaccagcacgtgaaggtggggactcacaggtgactgccggcgtaagtcggaggaaggtggggatgacgtcaaatcatcatgccctttatgtcctgggctacacacgtgctacaatgggcggtacaacgggaagcgagaccgcgaggtggagcgaaaccctgaaagccgttcgtagttcggattgcaggctgcaactcgcctgcatgaagccggaattgctagtaatcgcggatcagcatgccgcggtgaatccgttcccgggccttgtacacaccgcccgtcacaccacgagagttggcaacacccgaagtcggtgaggtaaccctttagggagccagccgccgaaggtggggtcgatgattggggtgaag

SEQ ID NO: 32 - DNA, 1523 bp, synthetic construct
  misc_feature 1..1523: note = synthetic construct
  variation 160
  variation 198..200
  source 1..1523: mol_type = other DNA
tagagtttgatcatggctcaggacgaacgctggcggcgtgcctaatacatgcaagtcgagcggacctcttcggaggtcagcggcggacgggtgaggaacacgtgggtaatctgcctttcagaccggaataacgcccggaaacgggcgctaatgccggatncgcccgcgagggggcatcttcttgcggggaaaggcccnnntgggccgctgagagaggagcccgcggcgcattagctcgttggcggggtaacggcccaccaaggcgacgatgcgtagccgacctgagagggtgaccggccacactgggactgagacacggcccagactcctacgggaggcagcagtagggaatcttccgcaatgggcgcaagcctgacggagcaacgccgcgtgagcgaggaaggccttcgggttgtaaagctctgttgctcggggagagcggcatggggagtggaaagtcccatgcgagacggtaccgagtgaggaagccccggctaactacgtgccagcagccgcggtaaaacgtagggggcgagcgttgtccggaatcactgggcgtaaagggtgcgtaggcggtcgagcaagtctggagtgaaagtccatggctcaaccatgggatggctctggaaactgcttgacttgagtgctggagaggcaaggggaattccacgtgtagcggtgaaatgcgtagagatgtggaggaataccagtggcgaaggcgccttgctggacagtgactgacgctgaggcacgaaagcgtggggagcaaacaggattagataccctggtagtccacgccgtaaacgatgagtgctaggtgttggggggacacaccccagtgccgaaggaaacccaataagcactccgcctggggagtacggtcgcaagactgaaactcaaaggaattgacgggggcccgcacaagcagtggagcatgtggtttaattcgaagcaacgcgaagaaccttaccagggcttgacatccctctgacaccctcagagatgaggggtcccttcggggcagaggagacaggtggtgcatggttgtcgtcagctcgtgtcgtgagatgttgggttcagtcccgcaacgagcgcaacccttgacctgtgttaccagcgcgaaagggcggggactcacaggtgactgccggcgtaagtcggaggaaggcggggatgacgtcaaatcatcatgcccctgatgtcctgggctacacacgtgctacaatgggcggtacaaagggaggcgaagccgcgaggcggagcgaaacccaaaaagccgctcgtagttcggattgcaggctgcaactcgcctgcatgaagccggaattgctagtaatcgcggatcagcatgccgcggtgaatacgttcccgggccttgtacacaccgcccgtcacaccacgagagtcggcaacacccgaagtcggtgaggtaaccccttatggggagccagccgccgaaggtggggtcgatgattggggtgaagtcgtaacaaggtagccgtaccggaaggtgcggctggatc

SEQ ID NO: 33 - DNA, 1463 bp, synthetic construct
  misc_feature 1..1463: note = synthetic construct
  variation 139
  variation 178
  source 1..1463: mol_type = other DNA
gacgaacgctggcggcgtgcctaatacatgcaagtcgagcggacctcttcggaggtcagcggcggacgggtgaggaacacgtgggtaatctgcctttcagaccggaataacgcccggaaacgggcgctaatgccggatncgcccgcgagggggcatcttcttgcggggaaaggcccanatgggccgctgagagaggagcccgcggcgcattagctcgttggcggggtaacggcccaccaaggcgacgatgcgtagccgacctgagagggtgaccggccacactgggactgagacacggcccagactcctacgggaggcagcagtagggaatcttccgcaatgggcgcaagcctgacggagcaacgccgcgtgagcgaggaaggccttcgggttgtaaagctctgttgctcggggagagcggcatggggagtggaaagtcccatgcgagacggtaccgagtgaggaagccccggctaactacgtgccagcagccgcggtaaaacgtagggggcgagcgttgtccggaatcactgggcgtaaagggtgcgtaggcggtcgagcaagtctggagtgaaagtccatggctcaaccatgggatggctctggaaactgcttgacttgagtgctggagaggcaaggggaattccacgtgtagcggtgaaatgcgtagagatgtggaggaataccagtggcgaaggcgccttgctggacagtgactgacgctgaggcacgaaagcgtggggagcaaacaggattagataccctggtagtccacgccgtaaacgatgagtgctaggtgttggggggacacaccccagtgccgaaggaaacccaataagcactccgcctggggagtacggtcgcaagactgaaactcaaaggaattgacgggggcccgcacaagcagtggagcatgtggtttaattcgaagcaacgcgaagaaccttaccagggcttgacatccctctgacaccctcagagatgaggggtcccttcggggcagaggagacaggtggtgcatggttgtcgtcagctcgtgtcgtgagatgttgggttcagtcccgcaacgagcgcaacccttgacctgtgttaccagcgcgaaagggcggggactcacaggtgactgccggcgtaagtcggaggaaggcggggatgacgtcaaatcatcatgcccctgatgtcctgggctacacacgtgctacaatgggcggtacaaagggaggcgaagccgcgaggcggagcgaaacccaaaaagccgctcgtagttcggattgcaggctgcaactcgcctgcatgaagccggaattgctagtaatcgcggatcagcatgccgcggtgaatacgttcccgggccttgtacacaccgcccgtcacaccacgagagtcggcaacacccgaagtcggtgaggtaaccccttatggggagccagccgccgaaggtggggtcgatgattggggtgaag

SEQ ID NO: 34 - DNA, 1438 bp, synthetic construct
  misc_feature 1..1438: note = synthetic construct
  source 1..1438: mol_type = other DNA
ctggcggcgtgcctaatacatgcaagtcgagcggacctcttcggaggtcagcggcggacgggtgaggaacacgtgggtaatctgcctttcagaccggaataacgcccggaaacgggcgctaatgccggatgcgcccgcgagggggcatcttcttgcggggaaaggcccagatgggccgctgagagaggagcccgcggcgcattagctcgttggcggggtaacggcccaccaaggcgacgatgcgtagccgacctgagagggtgaccggccacactgggactgagacacggcccagactcctacgggaggcagcagtagggaatcttccgcaatgggcgcaagcctgacggagcaacgccgcgtgagcgaggaaggccttcgggttgtaaagctctgttgctcggggagagcggcatggggagtggaaagtcccatgcgagacggtaccgagtgaggaagccccggctaactacgtgccagcagccgcggtaaaacgtagggggcgagcgttgtccggaatcactgggcgtaaagggtgcgtaggcggtcgagcaagtctggagtgaaagtccatggctcaaccatgggatggctctggaaactgcttgacttgagtgctggagaggcaaggggaattccacgtgtagcggtgaaatgcgtagagatgtggaggaataccagtggcgaaggcgccttgctggacagtgactgacgctgaggcacgaaagcgtggggagcaaacaggattagataccctggtagtccacgccgtaaacgatgagtgctaggtgttggggggacacaccccagtgccgaaggaaacccaataagcactccgcctggggagtacggtcgcaagactgaaactcaaaggaattgacgggggcccgcacaagcagtggagcatgtggtttaattcgaagcaacgcgaagaaccttaccagggcttgacatccctctgacaccctcagagatgaggggtcccttcggggcagaggagacaggtggtgcatggttgtcgtcagctcgtgtcgtgagatgttgggttcagtcccgcaacgagcgcaacccttgacctgtgttaccagcgcgaaagggcggggactcacaggtgactgccggcgtaagtcggaggaaggcggggatgacgtcaaatcatcatgcccctgatgtcctgggctacacacgtgctacaatgggcggtacaaagggaggcgaagccgcgaggcggagcgaaacccaaaaagccgctcgtagttcggattgcaggctgcaactcgcctgcatgaagccggaattgctagtaatcgcggatcagcatgccgcggtgaatacgttcccgggccttgtacacaccgcccgtcacaccacgagagtcggcaacacccgaagtcggtgaggtaaccccttatggggagccagccgccgaaggtggggt

SEQ ID NO: 35 - DNA, 1520 bp, synthetic construct
  misc_feature 1..1520: note = synthetic construct
  variation 476
  variation 1438
  source 1..1520: mol_type = other DNA
agagtttgatcctggctcaggacgaacgctggcggcgtgcctaatacatgcaagtcgagcggacctcttcggaggtcagcggcggacgggtgaggaacacgtgggtaatctgcctttcagaccggaataacgcccggaaacgggcgctaatgccggatacgcccgcgaggaggcatcttcttgcggggaaaggcccagttgggccgctgagagaggagcccgcggcgcattagctagttggtggggtaacggcccaccaaggcgacgatgcgtagccgacctgagagggtgaccggccacactgggactgagacacggcccagactcctacgggaggcagcagtagggaatcttccgcaatgggcgcaagcctgacggagcaacgccgcgtgagcgaagaaggccttcgggttgtaaagctctgttgctcggggagagcggcatggggagtggaaagccccatgcgagacggtacngagtgaggaagccccggctaactacgtgccagcagccgcggtaaaacgtagggggcgagcgttgtccggaatcactgggcgtaaagggtgcgtaggcggtcgagcaagtctggagtgaaagtccatggctcaaccatgggatggctctggaaactgcttgacttgagtgctggagaggcaaggggaattccacgtgtagcggtgaaatgcgtagagatgtggaggaataccagtggcgaaggcgccttgctggacagtgactgacgctgaggcacgaaagcgtggggagcaaacaggattagataccctggtagtccacgccgtaaacgatgagtgctaggtgttggggggacacaccccagtgccgaaggaaacccaataagcactccgcctggggagtacggtcgcaagactgaaactcaaaggaattgacgggggcccgcacaagcagtggagcatgtggtttaattcgaagcaacgcgaagaaccttaccagggcttgacatccctctgacgggtgcagagatgcaccttcccttcggggcagaggagacaggtggtgcatggttgtcgtcagctcgtgtcgtgagatgttgggttcagtcccgcaacgagcgcaacccttgacctgtgttaccagcgcgatagggcggggactcacaggtgactgccggcgtaagtcggaggaaggcggggatgacgtcaaatcatcatgcccctgatgtcctgggctacacacgtgctacaatgggcggtacaaagggaggcgaagccgcgaggcggagcgaaacccaaaaagccgctcgtagttcggattgcaggctgcaactcgcctgcatgaagccggaattgctagtaatcgcggatcagcatgccgcggtgaatacgttcccgggccttgtacacaccgcccgtcacaccacgagagtcggcaacacccgaagtcggtgaggtaaccccgnaaggggagccagccgccgaaggtggggtcgatgattggggtgaagtcgtaacaaggtagccgtaccggaaggtgcggttgga

SEQ ID NO: 36 - DNA, 1472 bp, synthetic construct
  misc_feature 1..1472: note = synthetic construct
  variation 1211
  source 1..1472: mol_type = other DNA
ggcgtgcctaatacatgcaagtcgagcggacctcttcggaggtcagcggcggacgggtgaggaacacgtgggtaatctgcctttcagaccggaataacgcccggaaacgggcgctaatgccggatacgcccgcgaggaggcatcttcttgcggggaaaggcccagttgggccgctgagagaggagcccgcggcgcattagctagttggtggggtaacggcccaccaaggcgacgatgcgtagccgacctgagagggtgaccggccacactgggactgagacacggcccagactcctacgggaggcagcagtagggaatcttccgcaatgggcgcaagcctgacggagcaacgccgcgtgagcgaagaaggccttcgggttgtaaagctctgttgctcggggagagcggcatggggagtggaaagccccatgcgagacggtaccgagtgaggaagccccggctaactacgtgccagcagccgcggtaaaacgtagggggcgagcgttgtccggaatcactgggcgtaaagggtgcgtaggcggtcgagcaagtctggagtgaaagtccatggctcaaccatgggatggctctggaaactgcttgacttgagtgctggagaggcaaggggaattccacgtgtagcggtgaaatgcgtagagatgtggaggaataccagtggcgaaggcgccttgctggacagtgactgacgctgaggcacgaaagcgtggggagcaaacaggattagataccctggtagtccacgccgtaaacgatgagtgctatgtgttggggggacacaccccagtgccgaaggaaacccaataagcactccgcctggggagtacggtcgcaagactgaaactcaaaggaattgacgggggcccgcacaagcagtggagcatgtggtttaattcgaagcaacgcgaagaaccttaccagggcttgacatccctctgacgggtgcagagatgcaccttcccttcggggcagaggagacaggtggtgcatggttgtcgtcagctcgtgtcgtgagatgttgggttcagtcccgcaacgagcgcaacccttgacctgtgttaccagcgcgatagggcggggactcacaggtgactgccggcgtaagtcggaggaaggcggggatgacgtcaaatcatcatgcccctgatgtcctgggctacacacgtgctacaatgggcggtacaaagggaggngaagccgcgaggcggagcgaaacccaaaaagccgctcgtagttcggattgcaggctgcaactcgcctgcatgaagccggaattgctagtaatcgcggatcagcatgccgcggtgaatacgttcccgggccttgtacacaccgcccgtcacaccacgagagtcggcaacacccgaagtcggtgaggtaaccccgaaaggggagccagccgccgaaggtggggtcgatgattggggtgaagtcgtaacaaggtagccgtaccg

SEQ ID NO: 37 - DNA, 1525 bp, Alicyclobacillus acidocaldarius
  source 1..1525: mol_type = genomic DNA
agagtttgatcctggctcaggacgaacgctggcggcgtgcctaatacatgcaagtcgagcggacctcttcggaggtcagcggcggacgggtgaggaacacgtgggtaatctgcctttcagaccggaataacgcccggaaacgggcgctaatgccggatacgccgcgaggaggcatcttcttgcggggaaaggcccgattgggccgctgagagaggagcccgcggcgcattagctcgttggtggggtaacggcccaccaaggcgacgatgcgtagccgacctgagagggtgaccggccacactgggactgagacacggcccagactcctacgggaggcagcagtagggaatcttccgcaatgggcgcaagcctgacggagcaacgccgcgtgagcgaagaaggccttcgggttgtaaagctctgttgctcggggagagcggcatggggagtggaaagccccatgcgagacggtaccgagtgaggaagccccggctaactacgtgccagcagccgcggtaaaacgtagggggcgagcgttgtccggaatcactgggcgtaaagggtgcgtaggcggtcgagcaagtctggagtgaaagtccatggctcaaccatgggatggctctggaaactgcttgacttgagtgctggagaggcaaggggaattccacgtgtagcggtgaaatgcgtagagatgtggaggaatacctgtggcgaaggcgccttgctggacagtgactgacgctgaggcacgaaagcgtggggagcaaacaggattagataccctggtagtccacgccgtaaacgatgagtgctaggtgttggggggacacaccccagtgccgaaggaaacccaataagcactccgcctggggagtacggtcgcaagactgaaactcaaaggaattgacgggggcccgcacaagcagtggagcatgtggtttaattcgaagcaacgcgaagaaccttaccagggcttgacatccctctgaccggtgcagagatgcaccttcccttcggggcagaggagacaggtggtgcatggttgtcgtcagctcgtgtcgtgagatgttgggttcagtcccgcaacgagcgcaacccttgacctgtgttaccagcgcgttgaggcggggactcacaggtgactgtcggcgtaagtcggaggaaggcggggatgacgtcaaatcatcatgcccctgatgtcctgggctacacacgtgctacaatgggcggtacaaagggaggcgaagccgcgaggcggagcgaaacccaaaaagccgctcgtagttcggattgcaggctgcaactcgcctgcatgaagccggaattgctagtaatcgcggatcagcatgccgcggtgaatacgttcccgggccttgtacacaccgcccgtcacaccacgagagtcggcaacacccgaagtcggtgaggtaaccccgaaaggggagccagccgccgaaggtggggtcgatgattggggtgaagtcgtaacaaggtagccgtaccggaaggtgcggctggatcacct

SEQ ID NO: 38 - DNA, 1507 bp, Alicyclobacillus acidocaldarius
  source 1..1507: mol_type = genomic DNA
cctggctcaggacgaacgctggcggcgtgcctaatacatgcaagtcgagcggacttttcggaggtcagcggcggacgggtgaggaacacgtgggtaatctgcctttcagaccggaataacgcccggaaacgggcgctaatgccggatacgcccgcgaggaggcatcttcttgcggggaaaggcccgattgggccgctgagagaggagcccgcggcgcattagctggttggcggggtaacggcccaccaaggcgacgatgcgtagccgacctgagagggtgaccggccacactgggactgagacacggcccagactcctacgggaggcagcagtagggaatcttccgcaatgggcgcaagcctgacggagcaacgccgcgtgagcgaagaaggccttcgggttgtaaagctctgttgctcggggagagcggcatggggagtggaaagccccatgcgagacggtaccgagtgaggaagccccggctaactacgtgccagcagccgcggtaaaacgtagggggcgagcgttgtccggaatcactgggcgtaaagggtgcgtaggcggtcgagcaagtctggagtgaaagtccatggctcaaccatgggatggctctggaaactgcttgacttcagtgctggagaggcaaggggaattccacgtgtagcggtgaaatgcgtagagatgtggaggaatacctgtggcgaaggcgccttgctggacagtgactgacgctgaggcacgaaagcgtggggagcaaacaggattagataccctggtagtccacgccgtaaacgatgagtgctaggtgttggggggacacaccccagtgccgaaggaaacccaataagcactccgcctggggagtacggtcgcaagactgaaactcaaaggaattgacgggggcccgcacaagcagtggagcatgtggtttaattcgaagcaacgcgaagaaccttaccagggcttgacatccctctgaccggtgcagagatgcaccttcccttcggggcagaggagacaggtggtgcatggttgtcgtcagctcgtgtcgtgagatgttgggttcagtcccgcaacgagcgcaacccttgacctgtgttaccagcgcgttgaggcggggactcacaggtgactgccggcgtaagtcggaggaaggcggggatgacgtcaaatcatcatgcccctgatgtcctgggctacacacgtgctacaatgggcggtacaaagggaggcgaagccgcgaggcggagcgaaacccaaaaagccgctcgtagttcggattgcaggctgcaactcgcctgcatgaagccggaattgctagtaatcgcggatcagcatgccgcggtgaatacgttcccgggccttgtacacaccgcccgtcacaccacgagagtcggcaacacccgaagtcggtgaggtaaccccgaaaggggagccagccgccgaaggtggggtcgatgattggggtgaagtcgtaacaaggtagccgtaccggaaggtgcggctg

SEQ ID NO: 39 - DNA, 1520 bp, Alicyclobacillus acidocaldarius
  variation 197
  variation 199
  source 1..1520: mol_type = genomic DNA
agagtttgatcctggctcaggacgaacgctggcggcgtgcctaatacatgcaagtcgagcggacctcttcggaggtcagcggcggacgggtgaggaacacgtgggtaatctgcctttcagaccggaataacgcccggaaacgggcgctaatgccggatacgcccgcgaggaggcatcttcttgcggggaaaggcccnantgggccgctgagagaggagcccgcggcgcattagctggttggcggggtaacggcccaccaaggcgacgatgcgtagccgacctgagagggtgaccggccacactgggactgagacacggcccagactcctacgggaggcagcagtagggaatcttccgcaatgggcgcaagcctgacggagcaacgccgcgtgagcgaagaaggccttcgggttgtaaagctctgttgctcggggagagcggcatggggagtggaaagccccatgcgagacggtaccgagtgaggaagccccggctaactacgtgccagcagccgcggtaaaacgtagggggcgagcgttgtccggaatcactgggcgtaaagggtgcgtaggcggtcgagcaagtctggagtgaaagtccatggctcaaccatgggatggctctggaaactgcttgacttgagtgctggagaggcaaggggaattccacgtgtagcggtgaaatgcgtagagatgtggaggaatacctgtggcgaaggcgccttgctggacagtgactgacgctgaggcacgaaagcgtggggagcaaacaggattagataccctggtagtccacgccgtaaacgatgagtgctaggtgttggggggacacaccccagtgccgaaggaaacccaataagcactccgcctggggagtacggtcgcaagactgaaactcaaaggaattgacgggggcccgcacaagcagtggagcatgtggtttaattcgaagcaacgcgaagaaccttaccagggcttgacatccctctgaccggtgcagagatgcaccttcccttcggggcagaggagacaggtggtgcatggttgtcgtcagctcgtgtcgtgagatgttgggttcagtcccgcaacgagcgcaacccttgacctgtgttaccagcgcgttgaggcggggactcacaggtgactgccggcgtaagtcggaggaaggcggggatgacgtcaaatcatcatgcccctgatgtcctgggctacacacgtgctacaatgggcggtacaaagggaggcgaagccgcgaggcggagcgaaacccaaaaagccgctcgtagttcggattgcaggctgcaactcgcctgcatgaagccggaattgctagtaatcgcggatcagcatgccgcggtgaatacgttcccgggccttgtacacaccgcccgtcacaccacgagagtcggcaacacccgaagtcggtgaggtaaccccgaaaggggagccagccgccgaaggtggggtcgatgattggggtgaagtcgtaacaaggtagccgtaccggaaggtgcggttgga

SEQ ID NO: 40 - DNA, 1463 bp, Alicyclobacillus acidocaldarius
  misc_difference 177: note = n is a, c, g, t or u
  misc_difference 978: note = n is a, c, g, t or u
  source 1..1463: mol_type = genomic DNA
gacgaacgctggcggcgtgcctaatacatgcaagtcgagcggacctcttcggaggtcagcggcggacgggtgaggaacacgtgggtaatctgcctttcagaccggaataacgcccggaaacgggcgctaatgccggatacgcccgcgaggaggcatcttcttgcggggaaaggcccnattgggccgctgagagaggagcccgcggcgcattagctggttggcggggtaacggcccaccaaggcgacgatgcgtagccgacctgagagggtgaccggccacactgggactgagacacggcccagactcctacgggaggcagcagtagggaatcttccgcaatgggcgcaagcctgacggagcaacgccgcgtgagcgaagaaggccttcgggttgtaaagctctgttgctcggggagagcggcatggggagtggaaagccccatgcgagacggtaccgagtgaggaagccccggctaactacgtgccagcagccgcggtaaaacgtagggggcgagcgttgtccggaatcactgggcgtaaagggtgcgtaggcggtcgagcaagtctggagtgaaagtccatggctcaaccatgggatggctctggaaactgcttgacttgagtgctggagaggcaaggggaattccacgtgtagcggtgaaatgcgtagagatgtggaggaatacctgtggcgaaggcgccttgctggacagtgactgacgctgaggcacgaaagcgtggggagcaaacaggattagataccctggtagtccacgccgtaaacgatgagtgctaggtgttggggggacacaccccagtgccgaaggaaacccaataagcactccgcctggggagtacggtcgcaagactgaaactcaaaggaattgacgggggcccgcacaagcagtggagcatgtggtttaattcgaagcaacgcgaagaaccttaccagggcttgacatccctctgaccggtgcngagatgcaccttcccttcggggcagaggagacaggtggtgcatggttgtcgtcagctcgtgtcgtgagatgttgggttcagtcccgcaacgagcgcaacccttgacctgtgttaccagcgcgttgaggcggggactcacaggtgactgccggcgtaagtcggaggaaggcggggatgacgtcaaatcatcatgcccctgatgtcctgggctacacacgtgctacaatgggcggtacaaagggaggcgaagccgcgaggcggagcgaaacccaaaaagccgctcgtagttcggattgcaggctgcaactcgcctgcatgaagccggaattgctagtaatcgcggatcagcatgccgcggtgaatacgttcccgggccttgtacacaccgcccgtcacaccacgagagtcggcaacacccgaagtcggtgaggtaaccccgaaaggggagccagccgccgaaggtggggtcgatgattggggtgaag

SEQ ID NO: 41 - DNA, 1458 bp, synthetic construct
  misc_feature 1..1458: note = synthetic construct
  source 1..1458: mol_type = other DNA
gacgaacgctggcggcgtgcctaatacatgcaagtcgagcgaacccttcggggtgagcggcggacgggtgagtaacacgtgggcaatctgcctatcagactggaataacactcggaaacgggtgctaatgccggatagttcaagggaaggcatcttccgttgaggaaagttgcaaatgcaacactgatagaggagcccgcggcgcattagctagttggtgaggtaacggcccaccaaggcgacgatgcgtagccgacctgagagggtgaccggccacactgggactgagacacggcccagactcctacgggaggcagcagtagggaatcttccgcaatgggcgaaagcctgacggagcaacgccgcgtgagcgaagaaggccttcgggttgtaaagctctgttgctcggggagagcgataaggagagtggaaagctccttaggagacggtaccgagtgaggaagccccggctaactacgtgccagcagccgcggtaatacgtagggggcaagcgttgtccggaatcactgggcgtaaagcgtgcgtaggcggttgtgtaagtctggagtgaaagtccaaggctcaaccttggtttgtctttggaaactgcataacttgagtgctggagaggcaaggggaattccacgtgtagcggtgaaatgcgtagatatgtggaggaataccagtggcgaaggcgccttgctggacagaaactgacgctgaggcacgaaagcgtggggagcaaacaggattagataccctggtagtccacgccgtaaacgatgagtgctaggtgttggggggatacacctcagtgccgaaggaaacccaataagcactccgcctggggagtacggtcgcaagactgaaactcaaaggaattgacgggggcccgcacaagcagtggagcatgtggtttaattcgaagcaacgcgaagaaccttaccagggcttgacatccctctgacgcatctagagataggtgttcccttcggggcagaggagacaggtggtgcatggttgtcgtcagctcgtgtcgtgagatgttgggttaagtcccgcaacgagcgcaacccttgatctgtgttaccagcacgtaaaggtggggactcacaggtgactgccggcgtaagtcggaggaaggcggggatgacgtcaaatcatcatgcccttgatgtcctgggctacacacgtgctacaatgggcggtacagagggaagcgaagccgcgaggtggagccaaacctagaaagccgttcgtagttcggattgcaggctgcaactcgcctgcatgaagccggaattgctagtaatcgcggatcagcatgccgcggtgaatccgttcccgggccttgtacacaccgcccgtcacaccacgagagtcggcaacacccgaagtcggtgaggcaacccgcaagggagccagccgccgaaggtggggttgatgattggggtgaag

SEQ ID NO: 42 - DNA, 1513 bp, synthetic construct
  misc_feature 1..1513: note = synthetic construct
  source 1..1513: mol_type = other DNA
agagtttgatcctggctcaggacgaacgctggcggcgtgcctaatacatgcaagtcgagcgaacccttcggggtgagcggcggacgggtgagtaacacgtgggcaatctgcctatcagactggaataacactcggaaacgggtgctaatgccggatagttcaagggaaggcatcttccgttgaggaaagttgcaaatgcaacactgatagaggagcccgcggcgcattagctagttggtgaggtaacggcccaccaaggcgacgatgcgtagccgacctgagagggtgaccggccacactgggactgagacacggcccagactcctacgggaggcagcagtagggaatcttccgcaatgggcgaaagcctgacggagcaacgccgcgtgagcgaagaaggccttcgggttgtaaagctctgttgctcggggagagcgataaggagagtggaaagctccttaggagacggtaccgagtgaggaagccccggctaactacgtgccagcagccgcggtaatacgtagggggcaagcgttgtccggaatcactgggcgtaaagcgtgcgtaggcggttgtgtaagtctggagtgaaagtccaaggctcaaccttggtttgtctttggaaactgcataacttgagtgctggagaggcaaggggaattccacgtgtagcggtgaaatgcgtagatatgtggaggaataccagtggcgaaggcgccttgctggacagaaactgacgctgaggcacgaaagcgtggggagcaaacaggattagataccctggtagtccacgccgtaaacgatgagtgctaggtgttggggggatacacctcagtgccgaaggaaacccaataagcactccgcctggggagtacggtcgcaagactgaaactcaaaggaattgacgggggcccgcacaagcagtggagcatgtggtttaattcgaagcaacgcgaagaaccttaccagggcttgacatccctctgacgcatctagagataggtgttcccttcggggcagaggagacaggtggtgcatggttgtcgtcagctcgtgtcgtgagatgttgggttaagtcccgcaacgagcgcaacccttgatctgtgttaccagcacgtaaaggtggggactcacaggtgactgccggcgtaagtcggaggaaggcggggatgacgtcaaatcatcatgcccttgatgtcctgggctacacacgtgctacaatgggcggtacagagggaagcgaagccgcgaggtggagccaaacctagaaagccgttcgtagttcggattgcaggctgcaactcgcctgcatgaagccggaattgctagtaatcgcggatcagcatgccgcggtgaatccgttcccgggccttgtacacaccgcccgtcacaccacgagagtcggcaacacccgaagtcggtgaggcaacccgcaagggagccagccgccgaaggtggggttgatgattggggtgaagtcgtaacaaggtagccgtatcggaaggtgcggctg

SEQ ID NO: 43 - DNA, 1459 bp, synthetic construct
  misc_feature 1..1459: note = synthetic construct
  variation 227
  variation 984
  source 1..1459: mol_type = other DNA
gacgaacgctggcggcgtgcctaatacatgcaagtcgagcggacatcttcggatgtcagcggcggacgggtgagtaacacgtgggcaatctgcctttcagaccggaataacactcggaaacgggtgctaatgccggataggtcacgaggaggcatcttcttgtgaggaaagctgcaaatgcagcgctgggagaggagcccgcggcgcattagctagttggtgaggtnacggctcaccaaggcgacgatgcgtagccgacctgagagggtgaccggccacactgggactgagacacggcccagactcctacgggaggcagcagtagggaatcttccgcaatgggcgaaagcctgacggagcaacgccgcgtgagcgaagaaggccttcgggttgtaaagctctgttgctcggggagagcgataaggagagtggaaagctccttaggagacggtaccgagtgaggaagccccggcaaactacgtgccagcagccgcggtaatacgtagggggcaagcgttgtccggaatcactgggcgtaaagcgtgcgtaggcggttgtgtaagtctggagtgaaagtccatggctcaaccatgggatggctttggaaactgcatgacttgagtgctggagaggcaaggggaattccacgtgtagcggtgaaatgcgtagatatgtggaggaataccagtggcgaaggcgccttgctggacagtgactgacgctgaggcacgaaagcgtggggagcaaacaggattagataccctggtagtccacgccgtaaacgatgagtgctaggtgttggggggatacacctcagtgccgaaggaaacccaataagcactccgcctggggagtacggtcgcaagactgaaactcaaaggaattgacgggggcccgcacaagcagtggagcatgtggtttaattcgaagcaacgcgaagaaccttaccagggcttgacatccctctgacaggtgcagagatgnaccttcccttcggggcagaggagacaggtggtgcatggttgtcgtcagctcgtgtcgtgagatgttgggttaagtcccgcaacgagcgcaacccttgacctgtgttaccagcacgtagaggtggggactcacaggtgactgccggcgtaagtcggaggaaggcggggatgacgtcaaatcatcatgccctttatgtcctgggctacacacgtgctacaatgggcggtacaacgggaagcgaagccgcgaggtggagccaacccagaaagccgttcgtagttcggattgcaggctgcaactcgcctgcatgaagccggaattgctagtaatcgcggatcagcatgccgcggtgaatccgttcccgggccttgtacacaccgcccgtcacaccacgagagtcggcaacacccgaagtcggtgaggtaacccttaggggagccagccgccgaaggtggggcggatgattggggtgaag

SEQ ID NO: 44 - DNA, 1518 bp, synthetic construct
  misc_feature 1..1518: note = synthetic construct
  variation 1005
  source 1..1518: mol_type = other DNA
tagagtttgatcatggctcaggacgaacgctggcggcgtgcctaatacatgcaagtcgagcggacatcttcggatgtcagcggcggacgggtgagtaacacgtgggcaatctgcctttcagaccggaataacactcggaaacgggtgctaatgccggataggtcacgaggaggcatcttcttgtgaggaaagctgcaaatgcagcgctgggagaggagcccgcggcgcattagctagttggtgaggtaacggctcaccaaggcgacgatgcgtagccgacctgagagggtgaccggccacactgggactgagacacggcccagactcctacgggaggcagcagtagggaatcttccgcaatgggcgaaagcctgacggagcaacgccgcgtgagcgaagaaggccttcgggttgtaaagctctgttgctcggggagagcgataaggagagtggaaagctccttaggagacggtaccgagtgaggaagccccggcaaactacgtgccagcagccgcggtaatacgtagggggcaagcgttgtccggaatcactgggcgtaaagcgtgcgtaggcggttgtgtaagtctggagtgaaagtccatggctcaaccatgggatggctttggaaactgcatgacttgagtgctggagaggcaaggggaattccacgtgtagcggtgaaatgcgtagatatgtggaggaataccagtggcgaaggcgccttgctggacagtgactgacgctgaggcacgaaagcgtggggagcaaacaggattagataccctggtagtccacgccgtaaacgatgagtgctaggtgttggggggatacacctcagtgccgaaggaaacccaataagcactccgcctggggagtacggtcgcaagactgaaactcaaaggaattgacgggggcccgcacaagcagtggagcatgtggtttaattcgaagcaacgcgaagaaccttaccagggcttgacatccctctgacaggtgcagagatgnaccttcccttcggggcagaggagacaggtggtgcatggttgtcgtcagctcgtgtcgtgagatgttgggttaagtcccgcaacgagcgcaacccttgacctgtgttaccagcacgtagaggtggggactcacaggtgactgccggcgtaagtcggaggaaggcggggatgacgtcaaatcatcatgccctttatgtcctgggctacacacgtgctacaatgggcggtacaacgggaagcgaagccgcgaggtggagccaaacccagaaagccgttcgtagttcggattgcaggctgcaactcgcctgcatgaagccggaattgctagtaatcgcggatcagcatgccgcggtgaatccgttcccgggccttgtacacaccgcccgtcacaccacgagagtcggcaacacccgaagtcggtgaggtaacccttaggggagccagccgccgaaggtggggcggatgattggggtgaagtcgtaacaaggtagccgtatcggaaggtgcggctgga

SEQ ID NO: 45 - DNA, 1517 bp, synthetic construct
  misc_feature 1..1517: note = synthetic construct
  variation 159
  variation 991
  source 1..1517: mol_type = other DNA
agagtttgatcctggctcaggacgaacgctggcggcgtgcctaatacatgcaagtcgtgcggacatcttcggatgtcagcggcggacgggtgagtaacacgtgggcaatctgcctttcagaccggaataacactcggaaacgggtgctaatgccggatnggtcacgaggaggcatcttcttgtgaggaaagttgcaaatgcagcgctgagagaggagcccgcggcgcattagctagttggtgaggtaacggctcaccaaggcgacgatgcgtagccgacctgagagggtgaccggccacactgggactgagacacggcccagactcctacgggaggcagcagtagggaatcttccgcaatgggcgaaagcctgacggagcaacgccgcgtgagcgaagaaggccttcgggttgtaaagctctgttgctcggggagagcgataaggagagtggaaagctccttaggagacggtaccgagtgaggaagccccggcaaactacgtgccagcagccgcggtaatacgtagggggcaagcgttgtccggaatcactgggcgtaaagcgtgcgtaggcggttgtgtaagtctggagtgaaagtccatggctcaaccatgggatggctttggaaactgcatgacttgagtgctggagaggcaaggggaattccacgtgtagcggtgaaatgcgtagatatgtggaggaataccagtggcgaaggcgccttgctggacagtgactgacgctgaggcacgaaagcgtggggagcaaacaggattagataccctggtagtccacgccgtaaacgatgagtgctaggtgttggggggatacacctcagtgccgaaggaaacccaataagcactccgcctggggagtacggtcgcaagactgaaactcaaaggaattgacgggggcccgcacaagcagtggagcatgtggtttaattcgaagcaacgcgaagaaccttaccagggcttgacatccctctgacnggtgcagagatgtaccttcccttcggggcagaggagacaggtggtgcatggttgtcgtcagctcgtgtcgtgagatgttgggttaagtcccgcaacgagcgcaacccttgacctgtgttaccagcacgtagaggtggggactcacaggtgactgccggcgtaagtcggaggaaggcggggatgacgtcaaatcatcatgccctttatgtcctgggctacacacgtgctacaatgggcggtacaacgggaagcgaagccgcgaggtggagccaaacccagaaagccgttcgtagttcggattgcaggctgcaactcgcctgcatgaagccggaattgctagtaatcgcggatcagcatgccgcggtgaatccgttcccgggccttgtacacaccgcccgtcacaccacgagagtcggcaacacccgaagtcggtgaggtaacccttaggggagccagccgccgaaggtggggcggatgattggggtgaagtcgtaacaaggtagccgtatcggaaggtgcggttgga

SEQ ID NO: 46 - DNA, 1485 bp, synthetic construct
  misc_feature 1..1485: note = synthetic construct
  source 1..1485: mol_type = other DNA
ctggcggcgtgcctaatacatgcaagtcgtgcggacatcttcggatgtcagcggcggacgggtgagtaacacgtgggcaatctgcctttcagaccggaataacactcggaaacgggtgctaatgccggataggtcacgaggaggcatcttcttgtgaggaaagttgcaaatgcagcgctgagagaggagcccgcggcgcattagctagttggtgaggtaacggctcaccaaggcgacgatgcgtagccgacctgagagggtgaccggccacactgggactgagacacggcccagactcctacgggaggcagcagtagggaatcttccgcaatgggcgaaagcctgacggagcaacgccgcgtgagcgaagaaggccttcgggttgtaaagctctgttgctcggggagagcgataaggagagtggaaagctccttaggagacggtaccgagtgaggaagccccggcaaactacgtgccagcagccgcggtaatacgtagggggcaagcgttgtccggaatcactgggcgtaaagcgtgcgtaggcggttgtgtaagtctggagtgaaagtccatggctcaaccatgggatggctttggaaactgcatgacttgagtgctggagaggcaaggggaattccacgtgtagcggtgaaatgcgtagatatgtggaggaataccagtggcgaaggcgccttgctggacagtgactgacgctgaggcacgaaagcgtggggagcaaacaggattagataccctggtagtccacgccgtaaacgatgagtgctaggtgttggggggatacacctcagtgccgaaggaaacccaataagcactccgcctggggagtacggtcgcaagactgaaactcaaaggaattgacgggggcccgcacaagcagtggagcatgtggtttaattcgaagcaacgcgaagaaccttaccagggcttgacatccctctgaccggtgcagagatgtaccttcccttcggggcagaggagacaggtggtgcatggttgtcgtcagctcgtgtcgtgagatgttgggttaagtcccgcaacgagcgcaacccttgacctgtgttaccagcacgtagaggtggggactcacaggtgactgccggcgtaagtcggaggaaggcggggatgacgtcaaatcatcatgccctttatgtcctgggctacacacgtgctacaatgggcggtacaacgggaagcgaagccgcgaggtggagccaaacccagaaagccgttcgtagttcggattgcaggctgcaactcgcctgcatgaagccggaattgctagtaatcgcggatcagcatgccgcggtgaatccgttcccgggccttgtacacaccgcccgtcacaccacgagagtcggcaacacccgaagtcggtgaggtaacccttaggggagccagccgccgaaggtggggcggatgattggggtgaagtcgtaacaaggtagccgtatcggaaggtgcggc

SEQ ID NO: 47 - DNA, 1421 bp, Alicyclobacillus acidoterrestris
  source 1..1421: mol_type = genomic DNA
gctcaggacgaacgctggcggcgtgcctaatacatgcaagtcgagcgagcccttcggggctagcggcggacgggtgagtaacacgtgggcaatctgcctttcagactggaataacactcggaaacgggtgctaatgccggataatacacgggtaggcatctacttgtgttgaaagatgcaactgcatcgctgagagaggagcccgcggcgcattagctagttggtgaggtaacggctcaccaaggcgacgatgcgtagccgacctgagagggtgaccggccacactgggactgagacacggcccagactcctacgggaggcagcagtagggaatcttccgcaatgggcgcaagcctgacggagcaacgccgcgtgagcgaagaaggccttcgggttgtaaagctctgttgctcggggagagcgacaaggagagtggaaagctccttgtgagacggtaccgagtgaggaagccccggctaactacgtgccagcagccgcggtaatacgtagggggcaagcgttgtccggaatcactgggcgtaaagcgtgcgtaggcggttgtgtaagtctgaagtgaaagtccaaggctcaaccttgggattgctttggaaactgcatgacttgagtgctggagaggcaaggggaattccacgtgtagcggtgaaatgcgtagatatgtggaggaataccagtggcgaaggcgccttgctggacagtgactgacgctgaggcacgaaagcgtggggagcaaacaggattagataccctggtagtccacgccgtaaacgatgagtgctaggtgttggggggacacaccccagtgccgaaggaaacccaataagcactccgcctggggagtacggtcgcaagactgaaactcaaaggaattgacgggggcccgcacaagcagtggagcatgtggtttaattcgaagcaacgcgaagaaccttaccagggcttgacatccctctgaccggtgcagagatgtaccttcccttcggggcagaggagacaggtggtgcatggttgtcgtcagctcgtgtcgtgagatgttgggttaagtcccgcaacgagcgcaacccttgatctgtgttaccagcacgtagaggtggggactcacaggtgactgccggcgtaagtcggaggaaggcggggatgacgtcaaatcatcatgccctttatgtcctgggctacacacgtgctacaatgggcggtacaacgggaagcgaagccgcgaggtggagcaaaacctaaaaagccgttcgtagttcggattgcaggctgcaactcgcctgcatgaagccggaattgctagtaatcgcggatcagcatgccgcggtgaatccgttcccgggccttgtacacaccgcccgtcacaccacgagagtcggcaacacccgaagtcggtgaggtaaccgtta

SEQ ID NO: 48 - DNA, 1514 bp, Alicyclobacillus acidoterrestris
  source 1..1514: mol_type = genomic DNA
agagtttgatcctggctcaggacgaacgctggcggcgtgcctaatacatgcaagtcgagcgagcccttcggggctagcggcggacgggtgagtaacacgtgggcaatctgcctttcagactggaataacactcggaaacgggtgctaatgccggataatacacgggtaggcatctacttgtgttgaaagatgcaactgcatcgctgagagaggagcccgcggcgcattagctagttggtgaggtaacggctcaccaaggcgacgatgcgtagccgacctgagagggtgaccggccacactgggactgagacacggcccagactcctacgggaggcagcagtagggaatcttccgcaatgggcgcaagcctgacggagcaacgccgcgtgagcgaagaaggccttcgggttgtaaagctctgttgctcggggagagcgacaaggagagtggaaagctccttgtgagacggtaccgagtgaggaagccccggctaactacgtgccagcagccgcggtaatacgtagggggcaagcgttgtccggaatcactgggcgtaaagcgtgcgtaggcggttgtgtaagtctgaagtgaaagtccaaggctcaaccttgggattgctttggaaactgcatgacttgagtgctggagaggcaaggggaattccacgtgtagcggtgaaatgcgtagatatgtggaggaataccagtggcgaaggcgccttgctggacagtgactgacgctgaggcacgaaagcgtggggagcaaacaggattagataccctggtagtccacgccgtaaacgatgagtgctaggtgttggggggacacaccccagtgccgaaggaaacccaataagcactccgcctggggagtacggtcgcaagactgaaactcaaaggaattgacgggggcccgcacaagcagtggagcatgtggtttaattcgaagcaacgcgaagaaccttaccagggcttgacatccctctgaccggtgcagagatgtaccttcccttcggggcagaggagacaggtggtgcatggttgtcgtcagctcgtgtcgtgagatgttgggttaagtcccgcaacgagcgcaacccttgatctgtgttaccagcacgtagaggtggggactcacaggtgactgccggcgtaagtcggaggaaggcggggatgacgtcaaatcatcatgccctttatgtcctgggctacacacgtgctacaatgggcggtacaacgggaagcgaagccgcgaggtggagcaaaacctaaaaagccgttcgtagttcggattgcaggctgcaactcgcctgcatgaagccggaattgctagtaatcgcggatcagcatgccgcggtgaatccgttcccgggccttgtacacaccgcccgtcacaccacgagagtcggcaacacccgaagtcggtgaggtaaccgttatggagccagccgccgaaggtggggttgatgattggggtgaagtcgtaacaaggtagccgtatcggaaggtgcggttgga

SEQ ID NO: 49 - DNA, 1456 bp, synthetic construct
  misc_feature 1..1456: note = synthetic construct
  source 1..1456: mol_type = other DNA
gacgaacgctggcggcgtgctaatacatgcaagtcgagcgaacccttcggggttagcggcggacgggtgagtaacacgtgggcaatctgcctgtcagactggaataacactcggaaacgggtgctaatgccggatgacacacgggaaggcatcttcctgtgttgaaaggtgcaactgcatcgctgatagaggagcccgcggcgcattagctagttggtgaggtaacggcccaccaaggcgacgatgcgtagccgacctgagagggtgaccggccacactgggactgagacacggcccagactcctacgggaggcagcagtagggaatcttccgcaatgggcgcaagcctgacggagcaacgccgcgtgagcgaagaaggccttcgggttgtaaagctctgttgctcggggagagcgacaaggagagtggaaagctccttgtgagacggtaccgagtgaggaagccccggctaactacgtgccagcagccacggtaatacgtagggggcaagcgttgtccggaatcactgggcgtaaagcgtgcgtaggcggttgtgtaagtctgaagtgaaagtccaaggctcaaccttgggattgctttggaaactgcataacttgagtgctggagaggcaaggggaattccacgtgtagcggtgaaatgcgtagatatgtggaggaataccagtggcgaaggcgccttgctggacagtgactgacgctgaggcacgaaagcgtggggagcaaacaggattagataccctggtagtccacgccgtaaacgatgagtgctaggtgttggggggacacaccccagtgccgaaggaaacccaataagcactccgcctggggagtacggtcgcaagactgaaactcaaaggaattgacgggggcccgcacaagcagtggagcatgtggtttaattcgaagcaacgcgaagaaccttaccagggcttgacatccctctgaccgggctagagatagtccttcccttcggggcagaggagacaggtggtgcatggttgtcgtcagctcgtgtcgtgagatgttgggttaagtcccgcaacgagcgcaacccttgacctgtgttaccagcacgtagtggtggggactcacaggtgactgccggcgtaagtcggaggaaggcggggatgacgtcaaatcatcatgccctttatgtcctgggctacacacgtgctacaatgggcggtacaacgggaagcgaagccgcgaggtggagcgaaacctaaaaagccgttcgtagttcggattgcaggctgcaactcgcctgcatgaagccggaattgctagtagtcgcggatcagcatgccgcggtgaatccgttcccgggccttgtacacaccgcccgtcacaccacgagagtcggcaacacccgaagtcggtgaggtaacctttcaggagccagccgccgaaggtggggttgatgattggggtgaag

SEQ ID NO: 50 - DNA, 1514 bp, synthetic construct
  misc_feature 1..1514: note = synthetic construct
  variation 71..72
  variation 988
  variation 1434..1435
  source 1..1514: mol_type = other DNA
agagtttgatcctggctcaggacgaacgctggcggcgtgcctaatacatgcaagtcgagcgaacccttcgnngttagcggcggacgggtgagtaacacgtgggcaatctgcctgtcagactggaataacactcggaaacgggtgctaatgccggatgacacacgggaaggcatcttcctgtgttgaaaggtgcaactgcatcgctgatagaggagcccgcggcgcattagctagttggtgaggtaacggcccaccaaggcgacgatgcgtagccgacctgagagggtgaccggccacactgggactgagacacggcccagactcctacgggaggcagcagtagggaatcttccgcaatgggcgcaagcctgacggagcaacgccgcgtgagcgaagaaggccttcgggttgtaaagctctgttgctcggggagagcgacaaggagagtggaaagctccttgtgagacggtaccgagtgaggaagccccggctaactacgtgccagcagccgcggtaatacgtagggggcaagcgttgtccggaatcactgggcgtaaagcgtgcgtaggcggttgtgtaagtctgaagtgaaagtccaaggctcaaccttgggattgctttggaaactgcataacttgagtgctggagaggcaaggggaattccacgtgtagcggtgaaatgcgtagatatgtggaggaataccagtggcgaaggcgccttgctggacagtgactgacgctgaggcacgaaagcgtggggagcaaacaggattagataccctggtagtccacgccgtaaacgatgagtgctaggtgttggggggacacaccccagtgccgaaggaaacccaataagcactccgcctggggagtacggtcgcaagactgaaactcaaaggaattgacgggggcccgcacaagcagtggagcatgtggtttaattcgaagcaacgcgaagaaccttaccagggcttgacatccctctgancgggctagagatagtccttcccttcggggcagaggagacaggtggtgcatggttgtcgtcagctcgtgtcgtgagatgttgggttaagtcccgcaacgagcgcaacccttgacctgtgttaccagcacgtagtggtggggactcacaggtgactgccggcgtaagtcggaggaaggcggggatgacgtcaaatcatcatgccctttatgtcctgggctacacacgtgctacaatgggcggtacaacgggaagcgaagccgcgaggtggagcgaaacctaaaaagccgttcgtagttcggattgcaggctgcaactcgcctgcatgaagccggaattgctagtaatcgcggatcagcatgccgcggtgaatccgttcccgggccttgtacacaccgcccgtcacaccacgagagtcggcaacacccgaagtcggtgaggtaaccttnnggagccagcccgccgaaggtggggttgatgattggggtgaagtcgtaacaaggtagccgtatcggaaggtgcggctgga

SEQ ID NO: 51 - DNA, 3507 bp, synthetic construct
  misc_feature 1..3507: note = synthetic construct
  source 1..3507: mol_type = other DNA
ctgttccatgagcttgagctgttggctcaatggcggctgagcgatattcaatcgtttcgctgccgccgtaatctgtccagcatccgcgatggtcacaaaatatctgagttggcgtaaatccatccgggcagcacctcatccatatgataatcatatgaccaatagatttaacaaatatttttaatatccataaacgtatactacactttacgtcgtgaaacaagatgccaccgcggaggcaacgaaggaaggtgggagatccgtgaaaatcattgtcggtattacaggcgccaccggtgcgatattcgggattcgactcctcgaattactgaacgacgcaggtgtggaaacacatctcgtggtatcaccgtgggcaaaatcgacaattccgtatgaaacgccatactcggtcgccgatgtattggcgatggccagctatacctattcacacaaagaccaggccgcgagaatatcgagcggatcatttcaggtcgacgggatgatcatcgcgccttgcagtatgaagaccctcgcgtcgattcgcatcggactcgcagacaacttgattacaagatctgccgacgtgatgctcaaagagcgcaagaaactgattttaatgacccgtgaaacgcctttaaacaccattcatctggaaaacatgctggcgctttcgcaaatgggcgccgtgatattcccaccgatgccagcgttctacaatcatccggaaacgctcgatgacatggtgaaccacattgtgtaccgtgtgatggatcaaatcggattaccacccgaagaggccaaccgctgggaaggcatcaaatcggaagtaagaactggaggttcacacgatggcatataaggatttccgagattttctggacactctgcgcaaagagggccagttgctcaccattgaagacgaggtcatgccggaacccgatttaggatctgcaggacgcgccatcaacaacgtcagcgacaaaagtcccgctctgctattcaccaatatcaacggatatcaaaacgcgtcggtcgccatgaacgtcatcggatcgtggcccaatcacgcgctgatgatggggcttcccaagaacacgccggttaaagagcagttttttgagttcgcccgccgttgggacaacttcccggttccggtcgagcgcatggacagcgcgcccttccaagaagtcgaaatcaatcaggacgtcaacttgttcgatatcctgcccctattccgcttgaaccaatacgacggtggcttctacatcgacaaggcgtgcgtcatctcgcgcgacccgtctgacccagacaacttcgacaaacaaaacgtcggcatttatcgcatccaggtcaagggccgcgatacacttggcatccagcctgtcccgcagcacgacatcgctgtgcacttacgcatggccgaagagcgcggagaaaacctccccgtcgcgctagctatcggctgtgaaccggttatcaccacgatggcagccgcaccaattggctacgaccagtcggaatacgaaatggccggtgccattcagggagagccgtacaaagtatttaccttgcctggcaccaacctggatgtaccttggggcgcggagtttgtcctcgaaggtgaaatcctggccggcgtgcgcgaatttgagggcccatttggcgagttcacaggtcactactctggtgggcgaagcatgcctgtcattcgcatcaaacgggtataccatcgcaagaacccgattttcgagcacctctacatcggtatgccatggacagagacggactacatggtcggcgtcaacacctgcgtgcccatgtatcagcaactcaaggacgcgttcccgaacgaaatcgtggccgtcaatgccatgtacacgcatggcctcgtcgccattatctcgaccaagaaacggtacggtgggtttgccaaagcagtcggcatgcgcgcaatgacgacaccgcacggcctcggatattgtaaactcgtgattgtagtggatgagacggttgatccattcaaccttccacaagtcatgtgggcgctgtcgaccaagatgcacccagctcacgacgtcatcatcttgccggacatgtccgttttaccactcgatcccggctccaacccagccggcatctcccacaagatgattctcgacgcgacgacaccggtggccccagagacacgcggccactactcgcaaccgctcgacgcaccattggcaacggagaagtgggagaaaattttccaagatctgctcagcaatcaataacgtacagaagaatcgataagggagtgtggttcacatgacaacttgcccacgctgtgattcaaccaaagccgaagcggtcgcaaagtctccggtccagggcgcttggacggtctactcttgtccggtctgtttttacacgtggcgttccacggaagccgataaatttaccgacccgagcaaatacgaaccgagcttcaaggtcaatcaggcggatattccgaacgccatccaggtgccgactgttccaccgcggataaaccgctgataaggggctgattgtatgttcgggaatctgcgcgaacttcctgacagcctactgacgttcttcagtggcgacgacctgtcgcaaaaacaacacgacgcgatgatgttattgacggtcaccgacgacctttggccgcatgcggccatggtcagtgtcggggaggtcgtcgccgtcgatagcacacacatgcgactcgccatgtggcctagtacgtcgacgacgcacaacctcgaacagcgagggcaagcgacgctcgtcctcgttcacgatgcaaaggcgtactacattcgcctccgcgtcgagccactggcgtcgaatccgaacgcccgccacaatctgcgccgcttcgcggccaccatcgagggcatccgtgaggacgtggcaaaatacgccgacctcacaagcggcattcgcatagaactaaaggatcccgcgagtgttctcgcccgctggcaagacaccatcgaggacacgctagagggttaggccaggcggcggtggatcgacaggcggatcgacaatcgggtcgacaggcggatcgacagtggacgtacagtatcgcacaatccgcggccgaatgacgcataaagctgcgatttctgcgcatcataagtcttgtaaggaggctgatgtgtgatggtgacgaccgcttaaggatgtctcaaaaccgcagttgacttacagaaaaggacggcttcgtgccgtcctttttcgtgtagggcggcatgggtggcggatcgggcggatggaccaggccctcgtgtcttattaagacatcgtcctcattctgacggcccataaggcagaaaatctgcactattgcgcctcgtgaccccggaaatcgccgatttttcgggcaataagacatttttcttgtctaaattccgccggatataaacaaagagcggaaatagcgcagaaatactgccctattttccgctctctccacattctactctccgccccaga